SEQUENCE LISTING (99 sequences)

SEQ ID NO: 1 - DNA, 36 bp, Artificial Sequence
  Synthetic
gcacgtagtataatcaactttgaaaaactgtaataa

SEQ ID NO: 2 - DNA, 36 bp, Artificial Sequence
  Synthetic
gcacgttctattatcaacttcgaaaaactataataa

SEQ ID NO: 3 - DNA, 36 bp, Artificial Sequence
  Synthetic
gcccgcagtattatcaatttcgaaaaattataataa

SEQ ID NO: 4 - DNA, 36 bp, Artificial Sequence
  Synthetic
gcgcgctctataattaacttcgaaaaactttaataa

SEQ ID NO: 5 - DNA, 36 bp, Artificial Sequence
  Synthetic
gcacgctccattattaactttgaaaaactttaataa

SEQ ID NO: 6 - DNA, 36 bp, Artificial Sequence
  Synthetic
gctcgctctatcatcaatttcgaaaaactttaataa

SEQ ID NO: 7 - DNA, 36 bp, Artificial Sequence
  Synthetic
gcacgtagtattattaacttcgaaaagttataataa

SEQ ID NO: 8 - DNA, 36 bp, Artificial Sequence
  Synthetic
gcacgttccatcattaactttgaaaaactataataa

SEQ ID NO: 9 - DNA, 36 bp, Artificial Sequence
  Synthetic
gctcgctcaatcatcaactttgaaaagctataataa

SEQ ID NO: 10 - DNA, 36 bp, Artificial Sequence
  Synthetic
gctcgctctatcatcaacttcgaaaaattgtaataa

SEQ ID NO: 11 - DNA, 36 bp, Artificial Sequence
  Synthetic
gctcgctctattatcaattttgaaaaattataataa

SEQ ID NO: 12 - DNA, 36 bp, Artificial Sequence
  Synthetic
gctcgtagtattattaatttcgaaaaattataataa

SEQ ID NO: 13 - DNA, 36 bp, Artificial Sequence
  Synthetic
gctcgttcgattatcaacttcgaaaaactgtaataa

SEQ ID NO: 14 - DNA, 36 bp, Artificial Sequence
  Synthetic
gcaagaagcatcatcaacttcgaaaaactgtaataa

SEQ ID NO: 15 - DNA, 36 bp, Artificial Sequence
  Synthetic
gcgcgttctattattaattttgaaaaattataataa

SEQ ID NO: 16 - protein, 10 aa, Artificial Sequence
  Synthetic
ARSIINFEKL

SEQ ID NO: 17 - DNA, 66 bp, Artificial Sequence
  Synthetic
gattataaagatcatgacggagactataaagaccatgacattgattacaaagacgacgatgacaaa

SEQ ID NO: 18 - DNA, 66 bp, Artificial Sequence
  Synthetic
gactataaagaccacgatggcgattataaagaccatgatattgactacaaagatgatgatgataag

SEQ ID NO: 19 - DNA, 66 bp, Artificial Sequence
  Synthetic
gattataaagatcatgatggcgactataaagatcatgatatcgattacaaagatgacgatgacaaa

SEQ ID NO: 20 - DNA, 66 bp, Artificial Sequence
  Synthetic
gactacaaagatcacgatggtgactacaaagatcacgacattgattataaagacgatgatgacaaa

SEQ ID NO: 21 - DNA, 66 bp, Artificial Sequence
  Synthetic
gattacaaagatcacgatggtgattataaggatcacgatattgattacaaagacgacgacgataaa

SEQ ID NO: 22 - DNA, 66 bp, Artificial Sequence
  Synthetic
gattacaaagatcacgatggcgattacaaagatcatgacattgactacaaagacgatgatgataaa

SEQ ID NO: 23 - DNA, 66 bp, Artificial Sequence
  Synthetic
gattacaaggatcatgatggtgattacaaagatcacgatatcgactacaaagatgatgacgataaa

SEQ ID NO: 24 - DNA, 66 bp, Artificial Sequence
  Synthetic
gactacaaagatcatgatggtgattacaaagatcatgacattgattataaagatgatgatgacaaa

SEQ ID NO: 25 - DNA, 66 bp, Artificial Sequence
  Synthetic
gattataaagaccatgatggtgattataaggatcatgatatcgattataaggatgacgacgataaa

SEQ ID NO: 26 - DNA, 66 bp, Artificial Sequence
  Synthetic
gattataaagatcacgatggcgattataaagaccacgatattgattataaagacgacgatgacaaa

SEQ ID NO: 27 - DNA, 66 bp, Artificial Sequence
  Synthetic
gactataaagaccacgatggtgattataaagatcacgacatcgactacaaagacgatgatgataaa

SEQ ID NO: 28 - DNA, 66 bp, Artificial Sequence
  Synthetic
gactacaaagatcacgacggcgattataaagatcacgatattgactataaagatgacgatgataaa

SEQ ID NO: 29 - DNA, 66 bp, Artificial Sequence
  Synthetic
gattataaagaccatgatggagattacaaagatcatgatattgactataaagacgacgacgataaa

SEQ ID NO: 30 - DNA, 66 bp, Artificial Sequence
  Synthetic
gattataaagatcacgatggtgactacaaagatcacgatatcgattataaagacgatgacgataaa

SEQ ID NO: 31 - DNA, 66 bp, Artificial Sequence
  Synthetic
gactacaaagatcacgatggtgattataaagaccatgatattgattacaaagatgatgatgacaaa

SEQ ID NO: 32 - protein, 22 aa, Artificial Sequence
  Synthetic
DYKDHDGDYKDHDIDYKDDDDK

SEQ ID NO: 33 - protein, 6 aa, Artificial Sequence
  Synthetic
GASGAS

SEQ ID NO: 34 - protein, 6 aa, Artificial Sequence
  Synthetic
GSAGSA

SEQ ID NO: 35 - protein, 4 aa, Artificial Sequence
  Synthetic
GGGG

SEQ ID NO: 36 - protein, 5 aa, Artificial Sequence
  Synthetic
GGGGS

SEQ ID NO: 37 - protein, 8 aa, Artificial Sequence
  Synthetic
VGKGGSGG

SEQ ID NO: 38 - protein, 5 aa, Artificial Sequence
  Synthetic
PAPAP

SEQ ID NO: 39 - protein, 5 aa, Artificial Sequence
  Synthetic
EAAAK

SEQ ID NO: 40 - protein, 6 aa, Artificial Sequence
  Synthetic
AYLAYL

SEQ ID NO: 41 - protein, 6 aa, Artificial Sequence
  Synthetic
LRALRA

SEQ ID NO: 42 - protein, 4 aa, Artificial Sequence
  Synthetic
RLRA

SEQ ID NO: 43 - protein, 32 aa, Artificial Sequence
  Synthetic
KENSISSMAPPASPPASPKTPIEKKHADEIDK

SEQ ID NO: 44 - protein, 19 aa, Artificial Sequence
  Synthetic
KENSISSMAPPASPPASPK

SEQ ID NO: 45 - protein, 14 aa, Artificial Sequence
  Synthetic
KTEEQPSEVNTGPR

SEQ ID NO: 46 - protein, 28 aa, Artificial Sequence
  Synthetic
KESVVDASESDLDSSMQSADESTPQPLK

SEQ ID NO: 47 - protein, 20 aa, Artificial Sequence
  Synthetic
KSEEVNASDFPPPPTDEELR

SEQ ID NO: 48 - protein, 33 aa, Artificial Sequence
  Synthetic
RGGRPTSEEFSSLNSGDFTDDENSETTEEEIDR

SEQ ID NO: 49 - protein, 17 aa, Artificial Sequence
  Synthetic
KQNTASTETTTTNEQPK

SEQ ID NO: 50 - protein, 17 aa, Artificial Sequence
  Synthetic
KQNTANTETTTTNEQPK

SEQ ID NO: 51 - protein, 19 aa, Artificial Sequence
  Synthetic
RSEVTISPAETPESPPATP

SEQ ID NO: 52 - protein, 28 aa, Artificial Sequence
  Synthetic
KASVTDTSEGDLDSSMQSADESTPQPLK

SEQ ID NO: 53 - protein, 20 aa, Artificial Sequence
  Synthetic
KNEEVNASDFPPPPTDEELR

SEQ ID NO: 54 - protein, 33 aa, Artificial Sequence
  Synthetic
RGGIPTSEEFSSLNSGDFTDDENSETTEEEIDR

SEQ ID NO: 55 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 56 - protein, 529 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 57 - protein, 441 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 58 - protein, 416 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 59 - protein, 441 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 60 - DNA, 1323 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgat

SEQ ID NO: 61 - protein, 633 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 62 - protein, 639 aa, Artificial Sequence
  Synthetic
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 63 - protein, 93 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 64 - protein, 200 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 65 - protein, 303 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 66 - protein, 370 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 67 - protein, 390 aa, Artificial Sequence
  Synthetic
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 68 - DNA, 1170 bp, Artificial Sequence
  Synthetic
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 69 - protein, 100 aa, Artificial Sequence
  Synthetic
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 70 - protein, 390 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 71 - DNA, 1170 bp, Artificial Sequence
  Synthetic
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 72 - protein, 226 aa, Artificial Sequence
  Synthetic
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 73 - protein, 5 aa, Artificial Sequence
  Synthetic
QDNKR

SEQ ID NO: 74 - protein, 11 aa, Artificial Sequence
  Synthetic
ECTGLAWEWWR

SEQ ID NO: 75 - protein, 11 aa, Artificial Sequence
  Synthetic
ESLLMWITQCR

SEQ ID NO: 76 - protein, 368 aa, Artificial Sequence
  Synthetic
MVTGWHRPTWIEIDRAAIRENIKNEQNKLPESVDLWAVVKANAYGHGIIEVARTAKEAGAKGFCVAILDEALALREAGFQDDFILVLGATRKEDANLAAKNHISLTVFREDWLENLTLEATLRIHLKVDSGMGRLGIRTTEEARRIEATSTNDHQLQLEGIYTHFATADQLETSYFEQQLAKFQTILTSLKKRPTYVHTANSAASLLQPQIGFDAIRFGISMYGLTPSTEIKTSLPFELKPALALYTEMVHVKELAPGDSVSYGATYTATEREWVATLPIGYADGLIRHYSGFHVLVDGEPAPIIGRVCMDQTIIKLPREFQTGSKVTIIGKDHGNTVTADDAAQYLDTINYEVTCLLNERIPRKYIH

SEQ ID NO: 77 - protein, 289 aa, Artificial Sequence
  Synthetic
MKVLVNNHLVEREDATVDIEDRGYQFGDGVYEVVRLYNGKFFTYNEHIDRLYASAAKIDLVIPYSKEELRELLEKLVAENNINTGNVYLQVTRGVQNPRNHVIPDDFPLEGVLTAAAREVPRNERQFVEGGTAITEEDVRWLRCDIKSLNLLGNILAKNKAHQQNALEAILHRGEQVTECSASNVSIIKDGVLWTHAADNLILNGITRQVIIDVAKKNGIPVKEADFTLTDLREADEVFISSTTIEITPITHIDGVQVADGKRGPITAQLHQYFVEEITRACGELEFAK

SEQ ID NO: 78 - DNA, 1107 bp, Artificial Sequence
  Synthetic
atggtgacaggctggcatcgtccaacatggattgaaatagaccgcgcagcaattcgcgaaaatataaaaaatgaacaaaataaactcccggaaagtgtcgacttatgggcagtagtcaaagctaatgcatatggtcacggaattatcgaagttgctaggacggcgaaagaagctggagcaaaaggtttctgcgtagccattttagatgaggcactggctcttagagaagctggatttcaagatgactttattcttgtgcttggtgcaaccagaaaagaagatgctaatctggcagccaaaaaccacatttcacttactgtttttagagaagattggctagagaatctaacgctagaagcaacacttcgaattcatttaaaagtagatagcggtatggggcgtctcggtattcgtacgactgaagaagcacggcgaattgaagcaaccagtactaatgatcaccaattacaactggaaggtatttacacgcattttgcaacagccgaccagctagaaactagttattttgaacaacaattagctaagttccaaacgattttaacgagtttaaaaaaacgaccaacttatgttcatacagccaattcagctgcttcattgttacagccacaaatcgggtttgatgcgattcgctttggtatttcgatgtatggattaactccctccacagaaatcaaaactagcttgccgtttgagcttaaacctgcacttgcactctataccgagatggttcatgtgaaagaacttgcaccaggcgatagcgttagctacggagcaacttatacagcaacagagcgagaatgggttgcgacattaccaattggctatgcggatggattgattcgtcattacagtggtttccatgttttagtagacggtgaaccagctccaatcattggtcgagtttgtatggatcaaaccatcataaaactaccacgtgaatttcaaactggttcaaaagtaacgataattggcaaagatcatggtaacacggtaacagcagatgatgccgctcaatatttagatacaattaattatgaggtaacttgtttgttaaatgagcgcatacctagaaaatacatccattag

SEQ ID NO: 79 - DNA, 870 bp, Artificial Sequence
  Synthetic
atgaaagtattagtaaataaccatttagttgaaagagaagatgccacagttgacattgaagaccgcggatatcagtttggtgatggtgtatatgaagtagttcgtctatataatggaaaattctttacttataatgaacacattgatcgcttatatgctagtgcagcaaaaattgacttagttattccttattccaaagaagagctacgtgaattacttgaaaaattagttgccgaaaataatatcaatacagggaatgtctatttacaagtgactcgtggtgttcaaaacccacgtaatcatgtaatccctgatgatttccctctagaaggcgttttaacagcagcagctcgtgaagtacctagaaacgagcgtcaattcgttgaaggtggaacggcgattacagaagaagatgtgcgctggttacgctgtgatattaagagcttaaaccttttaggaaatattctagcaaaaaataaagcacatcaacaaaatgctttggaagctattttacatcgcggggaacaagtaacagaatgttctgcttcaaacgtttctattattaaagatggtgtattatggacgcatgcggcagataacttaatcttaaatggtatcactcgtcaagttatcattgatgttgcgaaaaagaatggcattcctgttaaagaagcggatttcactttaacagaccttcgtgaagcggatgaagtgttcatttcaagtacaactattgaaattacacctattacgcatattgacggagttcaagtagctgacggaaaacgtggaccaattacagcgcaacttcatcaatattttgtagaagaaatcactcgtgcatgtggcgaattagagtttgcaaaataa

SEQ ID NO: 80 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYGKETPDGIKITLDNLTMQELGYSSGIAHSSAVSRIISKLKQEKVIVYKNSCFYVQNLDYLKRYAPKLDEWFYLACPATWGKLN

SEQ ID NO: 81 - DNA, 714 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgatttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcttgattatctcaaaagatatgcccctaaattagatgaatggttttatttagcatgtcctgctacttggggaaaattaaattaa

SEQ ID NO: 82 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNVFSINGKLGSICGQLLILTYVYGKETPDGIKITLDNLTMQELGYSSGIAHSSAVSRIISKLKQEKVIVYKNSCFYVQNRDYLKRYAPKLDEWFYLACPATWGKLN

SEQ ID NO: 83 - DNA, 713 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgttttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatctgattatctcaaaagatatgcccctaaattagatgaatggttttatttagcatgtcctgctacttggggaaaattaaattaa

SEQ ID NO: 84 - DNA, 12 bp, Artificial Sequence
  Synthetic
ggtggtggagga

SEQ ID NO: 85 - DNA, 12 bp, Artificial Sequence
  Synthetic
ggtggaggtgga

SEQ ID NO: 86 - DNA, 12 bp, Artificial Sequence
  Synthetic
ggtggaggaggt

SEQ ID NO: 87 - DNA, 12 bp, Artificial Sequence
  Synthetic
ggaggtggtgga

SEQ ID NO: 88 - DNA, 12 bp, Artificial Sequence
  Synthetic
ggaggaggtggt

SEQ ID NO: 89 - DNA, 12 bp, Artificial Sequence
  Synthetic
ggaggtggaggt

SEQ ID NO: 90 - DNA, 12 bp, Artificial Sequence
  Synthetic
ggaggaggaggt

SEQ ID NO: 91 - DNA, 12 bp, Artificial Sequence
  Synthetic
ggaggaggtgga

SEQ ID NO: 92 - DNA, 12 bp, Artificial Sequence
  Synthetic
ggaggtggagga

SEQ ID NO: 93 - DNA, 12 bp, Artificial Sequence
  Synthetic
ggtggaggagga

SEQ ID NO: 94 - DNA, 12 bp, Artificial Sequence
  Synthetic
ggaggaggagga

SEQ ID NO: 95 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKEATGLAWEAARTVIDDRNLPLVKNRNISIWGTTLYPKYSNKVDNPIE

SEQ ID NO: 96 - protein, 11 aa, Artificial Sequence
  Synthetic
EATGLAWEAAR

SEQ ID NO: 97 - protein, 25 aa, Artificial Sequence
  Synthetic
MKKIMLVFITLILVSLPIAQQTEAK

SEQ ID NO: 98 - protein, 29 aa, Artificial Sequence
  Synthetic
MGLNRFMRAMMVVFITANCITINPDIIFA

SEQ ID NO: 99 - protein, 21 aa, Artificial Sequence
  Synthetic
DYKDHDGDYKDHDIDYKDDDK

(19) United States
(12) Patent Application Publication
(10) Pub. No.: US 2021/0003558 A1
(43) Pub. Date: Jan. 7, 2021

(54) COMPOSITIONS AND METHODS FOR EVALUATING ATTENUATION AND INFECTIVITY OF LISTERIA STRAINS

(71) Applicant: ADVAXIS, INC., Princeton, NJ (US)

(72) Inventors: Poonam MOLLI, North Brunswick, NJ (US); Anu WALLECHA, Yardley, PA (US)

(73) Assignee: ADVAXIS, INC., Princeton, NJ (US)

(21) Appl. No.: 16/979,436

(22) PCT Filed: Mar. 8, 2019
(86) PCT No.: PCT/US2019/021303

§ 371 Date: Sep. 9, 2020

Related U.S. Application Data
(60) Provisional application No. 62/640,855, filed on Mar. 9, 2018.

(51) Int. Cl. G01N 33/50; C12N 1/20; G01N 33/483

(52) U.S. Cl. CPC G01N 33/5014; G01N 33/5055; G01N 33/4833; C12N 1/20

(57) ABSTRACT

Methods and compositions are provided for assessing attenuation and/or infectivity of bacteria or Listeria strains, such as Listeria monocytogenes.

### CROSS-REFERENCE TO RELATED APPLICATIONS

This application claims the benefit of U.S. Application No. 62/640,855, filed Mar. 9, 2018, which is herein incorporated by reference in its entirety for all purposes.

### REFERENCE TO A SEQUENCE LISTING SUBMITTED AS A TEXT FILE VIA EFS WEB

The Sequence Listing written in file 528092_SeqListing_ST25.txt is 89 kilobytes, was created on Feb. 25, 2019, and is hereby incorporated by reference.

### BACKGROUND

Listeria monocytogenes (Lm) is a gram-positive, non-spore forming bacterial organism that is responsible for listeriosis in humans. In order to use Lm-based immunotherapies such as cancer immunotherapies, the bacteria are bio-engineered to be attenuated such that they can be used to deliver tumor-specific antigen and generate antigen-specific immune response but not cause listeriosis. Primary macrophages can be used to assess the ability of Lm-based immunotherapies to infect and replicate in the cytosol. However, better methods are needed to assess attenuation and infectivity of Listeria strains.

### SUMMARY

Methods and compositions are provided for assessing attenuation and/or infectivity of bacteria or Listeria strains, such as Listeria monocytogenes. In some aspects, provided are methods for assessing attenuation or infectivity of a test Listeria strain. Such methods can comprise, for example: (a) infecting differentiated THP-1 cells with the test Listeria strain, wherein the THP-1 cells have been differentiated into macrophages prior to infecting with the test Listeria strain; (b) lysing the THP-1 cells and plating the lysate on agar; and; and (c) counting the Listeria that have multiplied inside the THP-1 cells by growth on the agar.

### BRIEF DESCRIPTION OF THE DRAWINGS

FIG. 1A. Graph illustrating bacterial growth rates and doubling times for reference standard and wild type control plotted as time versus viable cell counts (VCC) for qualification assay 3.

FIG. 1B. Graph illustrating bacterial growth rates and doubling times for reference standard and wild type control plotted as time versus viable cell counts (VCC) for qualification assay 4.

FIG. 1C. Graph illustrating bacterial growth rates and doubling times for reference standard and wild type control plotted as time versus viable cell counts (VCC) for qualification assay 5.

FIG. 2A. Graph illustrating bacterial growth rates and doubling times for wild type plotted as time versus viable cell counts (VCC) showing inter-assay comparison.

FIG. 2B. Graph illustrating bacterial growth rates and doubling times for reference standard ADXS11-001 plotted as time versus viable cell counts (VCC) showing inter-assay comparison.

FIG. 3. Graph illustrating the raw count information observed at time points: p-2, p0, p1, p3, and p5.

FIG. 4. Graph illustrating the ratio of the count at p-2 to that seen at p0.

FIG. 5. Graph illustrating the ratio of the count at p-2 to that seen at p0 as a ratio to wild type.

FIG. 6. Graph illustrating the ratio of the count at p3 and p % to that seen at p0.

FIG. 7. Graph illustrating the ratio of the count at p3 and p % to that seen at p0 relative to wild type.

FIG. 8. Graph illustrating the ratio of the count at p3 and p % to that seen at p0 relative to wild type by data run.

FIG. 9. Graph illustrating the impact of the number of passages in the proportional decrease in counts from p-2 to p0 relative to wild type.

FIG. 10. Graph illustrating regression analysis was used to evaluate the impact of the number of passages.

FIG. 11. Graph illustrating the relationship between the two resulting variables for each curve in FIG. 3.

### DEFINITIONS

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The term “fusion protein” refers to a protein comprising two or more peptides linked together by peptide bonds or other chemical bonds. The peptides can be linked together directly by a peptide or other chemical bond. For example, a chimeric molecule can be recombinantly expressed as a single-chain fusion protein. Alternatively, the peptides can be linked together by a “linker” such as one or more amino acids or another suitable linker between the two or more peptides.

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” refers to a process of modifying a nucleic acid sequence for enhanced expression in particular host cells by replacing at least one codon of the native sequence with a codon that is more frequently or most frequently used in the genes of the host cell while maintaining the native amino acid sequence. For example, a polynucleotide encoding a fusion polypeptide can be modified to substitute codons having a higher frequency of usage in a given Listeria cell or any other host cell as compared to the naturally occurring nucleic acid sequence. Codon usage tables are readily available, for example, at the “Codon Usage Database.” The optimal codons utilized by L. monocytogenes for each amino acid are shown US 2007/0207170, herein incorporated by reference in its entirety for all purposes. These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “plasmid” or “vector” includes any known delivery vector including a bacterial delivery vector, a viral vector delivery vector, a peptide immunotherapy delivery vector, a DNA immunotherapy delivery vector, an episomal plasmid, an integrative plasmid, or a phage vector. The term “vector” refers to a construct which is capable of delivering, and, optionally, expressing, one or more fusion polypeptides in a host cell.

The term “episomal plasmid” or “extrachromosomal plasmid” refers to a nucleic acid vector that is physically separate from chromosomal DNA (i.e., episomal or extrachromosomal and does not integrated into a host cell's genome) and replicates independently of chromosomal DNA. A plasmid may be linear or circular, and it may be single-stranded or double-stranded. Episomal plasmids may optionally persist in multiple copies in a host cell's cytoplasm (e.g., Listeria), resulting in amplification of any genes of interest within the episomal plasmid.

The term “genomically integrated” refers to a nucleic acid that has been introduced into a cell such that the nucleotide sequence integrates into the genome of the cell and is capable of being inherited by progeny thereof. Any protocol may be used for the stable incorporation of a nucleic acid into the genome of a cell.

The term “stably maintained” refers to maintenance of a nucleic acid molecule or plasmid in the absence of selection (e.g., antibiotic selection) for at least 10 generations without detectable loss. For example, the period can be at least 15 generations, 20 generations, at least 25 generations, at least 30 generations, at least 40 generations, at least 50 generations, at least 60 generations, at least 80 generations, at least 100 generations, at least 150 generations, at least 200 generations, at least 300 generations, or at least 500 generations. Stably maintained can refer to a nucleic acid molecule or plasmid being maintained stably in cells in vitro (e.g., in culture), being maintained stably in vivo, or both.

An “open reading frame” or “ORF” is a portion of a DNA which contains a sequence of bases that could potentially encode a protein. As an example, an ORF can be located between the start-code sequence (initiation codon) and the stop-codon sequence (termination codon) of a gene.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety.

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “wild type” refers to entities having a structure and/or activity as found in a normal (as contrasted with mutant, diseased, altered, or so forth) state or context. Wild type gene and polypeptides often exist in multiple different forms (e.g., alleles).

The term “isolated” with respect to proteins and nucleic acid refers to proteins and nucleic acids that are relatively purified with respect to other bacterial, viral or cellular components that may normally be present in situ, up to and including a substantially pure preparation of the protein and the polynucleotide. The term “isolated” also includes proteins and nucleic acids that have no naturally occurring counterpart, have been chemically synthesized and are thus substantially uncontaminated by other proteins or nucleic acids, or has been separated or purified from most other cellular components with which they are naturally accompanied (e.g., other cellular proteins, polynucleotides, or cellular components).

“Exogenous” or “heterologous” molecules or sequences are molecules or sequences that are not normally expressed in a cell or are not normally present in a cell in that form. Normal presence includes presence with respect to the particular developmental stage and environmental conditions of the cell. An exogenous or heterologous molecule or sequence, for example, can include a mutated version of a corresponding endogenous sequence within the cell or can include a sequence corresponding to an endogenous sequence within the cell but in a different form (i.e., not within a chromosome). An exogenous or heterologous molecule or sequence in a particular cell can also be a molecule or sequence derived from a different species than a reference species of the cell or from a different organism within the same species. For example, in the case of a Listeria strain expressing a heterologous polypeptide, the heterologous polypeptide could be a polypeptide that is not native or endogenous to the Listeria strain, that is not normally expressed by the Listeria strain, from a source other than the Listeria strain, derived from a different organism within the same species.

In contrast, “endogenous” molecules or sequences or “native” molecules or sequences are molecules or sequences that are normally present in that form in a particular cell at a particular developmental stage under particular environmental conditions.

The term “variant” refers to an amino acid or nucleic acid sequence (or an organism or tissue) that is different from the majority of the population but is still sufficiently similar to the common mode to be considered to be one of them (e.g., splice variants).

The term “isoform” refers to a version of a molecule (e.g., a protein) with only slight differences compared to another isoform, or version (e.g., of the same protein). For example, protein isoforms may be produced from different but related genes, they may arise from the same gene by alternative splicing, or they may arise from single nucleotide polymorphisms.

The term “fragment” when referring to a protein means a protein that is shorter or has fewer amino acids than the full length protein. The term “fragment” when referring to a nucleic acid means a nucleic acid that is shorter or has fewer nucleotides than the full length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “analog” when referring to a protein means a protein that differs from a naturally occurring protein by conservative amino acid differences, by modifications which do not affect amino acid sequence, or by both.

The term “functional” refers to the innate ability of a protein or nucleic acid (or a fragment, isoform, or variant thereof) to exhibit a biological activity or function. Such biological activities or functions can include, for example, the ability to elicit an immune response when administered to a subject. Such biological activities or functions can also include, for example, binding to an interaction partner. In the case of functional fragments, isoforms, or variants, these biological functions may in fact be changed (e.g., with respect to their specificity or selectivity), but with retention of the basic biological function.

The terms “immunogenicity” or “immunogenic” refer to the innate ability of a molecule (e.g., a protein, a nucleic acid, an antigen, or an organism) to elicit an immune response in a subject when administered to the subject. Immunogenicity can be measured, for example, by a greater number of antibodies to the molecule, a greater diversity of antibodies to the molecule, a greater number of T-cells specific for the molecule, a greater cytotoxic or helper T-cell response to the molecule, and the like.

The term “antigen” is used herein to refer to a substance that, when placed in contact with a subject or organism (e.g., when present in or when detected by the subject or organism), results in a detectable immune response from the subject or organism. An antigen may be, for example, a lipid, a protein, a carbohydrate, a nucleic acid, or combinations and variations thereof. For example, an “antigenic peptide” refers to a peptide that leads to the mounting of an immune response in a subject or organism when present in or detected by the subject or organism. For example, such an “antigenic peptide” may encompass proteins that are loaded onto and presented on MHC class I and/or class II molecules on a host cell's surface and can be recognized or detected by an immune cell of the host, thereby leading to the mounting of an immune response against the protein. Such an immune response may also extend to other cells within the host, such as diseased cells (e.g., tumor or cancer cells) that express the same protein.

The term “epitope” refers to a site on an antigen that is recognized by the immune system (e.g., to which an antibody binds). An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

The term “mutation” refers to the any change of the structure of a gene or a protein. For example, a mutation can result from a deletion, an insertion, a substitution, or a rearrangement of chromosome or a protein. An “insertion” changes the number of nucleotides in a gene or the number of amino acids in a protein by adding one or more additional nucleotides or amino acids. A “deletion” changes the number of nucleotides in a gene or the number of amino acids in a protein by reducing one or more additional nucleotides or amino acids.

A “frameshift” mutation in DNA occurs when the addition or loss of nucleotides changes a gene's reading frame. A reading frame consists of groups of 3 bases that each code for one amino acid. A frameshift mutation shifts the grouping of these bases and changes the code for amino acids. The resulting protein is usually nonfunctional. Insertions and deletions can each be frameshift mutations.

A “missense” mutation or substitution refers to a change in one amino acid of a protein or a point mutation in a single nucleotide resulting in a change in an encoded amino acid. A point mutation in a single nucleotide that results in a change in one amino acid is a “nonsynonymous” substitution in the DNA sequence. Nonsynonymous substitutions can also result in a “nonsense” mutation in which a codon is changed to a premature stop codon that results in truncation of the resulting protein. In contrast, a “synonymous” mutation in a DNA is one that does not alter the amino acid sequence of a protein (due to codon degeneracy).

The term “somatic mutation” includes genetic alterations acquired by a cell other than a germ cell (e.g., sperm or egg). Such mutations can be passed on to progeny of the mutated cell in the course of cell division but are not inheritable. In contrast, a germinal mutation occurs in the germ line and can be passed on to the next generation of offspring.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### DETAILED DESCRIPTION

### I. Overview

Disclosed herein is are cell-based assays using differentiated THP-1 cells to analyze intracellular growth of Listeria-based immunotherapies. Such assays can be used, for example, to evaluate attenuation of recombinant Listeria strains compared to wild type Listeria or to assess potency or infectivity of recombinant Listeria strains.

As one specific example, ADXS11-001 is a recombinant Listeria monocytogenes (Lm) strain attenuated due to the irreversible deletion of prfA in the genome and, further, its complementation with mutated prfA gene (D133V). The prfA gene regulates the transcription of several virulence genes such as hly (Listeriolysin O or LLO), actA (Actin nucleator A), plcA (phospholipase A), and plcB (phospholipase B), that are required for in vivo intracellular growth and survival of Lm. The complementation with mutated prfA in ADXS11-001 causes a reduction in the expression of the virulence genes. The plasmid in the ADXS11-001 immunotherapy also contains human papillomavirus protein E7 fused to truncated Listeriolysin O (tLLO)) under the control of the hly promoter. In order to evaluate attenuation of ADXS11-001, infection and replication is assessed in a macrophage cell infection assay using wild type Lm as control.

The biological activity of ADXS11-001 relies upon uptake of ADXS11-001 by antigen presenting cells (APC) such as macrophages and dendritic cells, its escape from phagolysosome, intracellular replication in the cytosol of APC, expression of tLLO-E7, processing, and presentation of tLLO-E7 on surface of APC to stimulate E7-specific cytotoxic T cell response. Using differentiated THP-1 cells is a superior alternative to using primary macrophages to monitor the ability of ADXS11-001 to infect and replicate in the cytosol of macrophage. The method is also advantageous in that it is quantitative.

### II. Methods for Evaluating Attenuation and Infectivity of Listeria

Methods and compositions are provided for assessing attenuation and/or infectivity of bacteria. In some embodiments, the bacteria is a Listeria strain. In some embodiments, the Listeria strain is a Listeria monocytogenes strain. In some embodiments, the L. monocytogenes strain is a mutant, recombinant, or attenuated L. monocytogenes strain. Examples of recombinant Listeria strains that can be used in such methods are provided in more detail elsewhere herein. Such methods utilize macrophage cell lines or macrophage-like cell lines with macrophage phenotypes. Such cells can be immortalized cells. For example, the cell line can be a human monocyte cell line such as THP-1 cells. THP-1 designates a spontaneously immortalized monocyte-like cell line, derived from the peripheral blood of a childhood case of acute monocytic leukemia (M5 subtype). THP-1 cells can be differentiated into macrophage-like cells using, for example, phorbol 12-myristate 13-acetate (commonly known as PMA or TPA).

In some embodiments, the methods comprise: (a) infecting differentiated THP-1 cells with a test Listeria strain, wherein the THP-1 cells have been differentiated into macrophages prior to infecting with the test Listeria strain; (b) lysing the THP-1 cells and plating the lysate on agar; and (c) counting the Listeria that have multiplied inside the THP-1 cells by growth on the agar. The differentiated THP-1 cells can be grown as adherent cells. Other macrophage-like cells can also be used. Other macrophage-like immortalized cells and/or cell lines can also be used.

In some embodiments, the methods further comprise differentiating the THP-1 cells into macrophages. For example, such differentiation can be accomplished using phorbol 12-myristate 13-acetate (PMA) prior to step (a) as disclosed elsewhere herein. In some embodiments, prior to differentiation, the passage number for the THP-1 cells is less than 32.

In some embodiments, step (a) comprises infecting the differentiated THP-1 cells at a multiplicity of infection (MOI) of 1:1. However, any suitable multiplicity of infection can be used.

Optionally, such methods can further comprise killing all the Listeria not taken up by the THP-1 cells in between steps (a) and (b). For example, the killing can be performed using an antibiotic such as gentamicin.

Optionally, the lysing step (b) is performed at 3 hours post-infection. However, the lysing step can be performed at other time points as well, such as 1, 2, 3, 4, 5, 6, 7, 8, 9, or 10 hours post-infection.

In some embodiments, infecting differentiated THP-1 cells with a bacteria strain comprises incubating the bacteria with the differentiated THP-1 cells for 1-5 h, 2-3 h, 1 h, 2 h, 3 h, 2 h±60 min, 2 h±50 min, 2 h±40 min, 2 h±30 min, 2 h±25 min, 2 h±20 min, 2 h 15 min, 2 h 10 min, 2 h 5 min, or 2 h 3 min. In some embodiments, the bacteria is a Listeria. In some embodiments, the Listeria is L. monocytogenes. In some embodiments, the L. monocytogenes is attenuated relative to wild-type L. monocytogenes. In some embodiments, an inoculating media containing the bacteria is added to the differentiated THP-1 cells.

In some embodiments, the infecting step further comprises one or more washing steps and/or a killing step. A washing step can comprise removing bacteria-containing media from the THP-1 cells and optionally rinsing the THP-1 cells, thereby remove bacteria that have not infected the THP-1 cells. The washing step, if used, can be performed following incubation of the bacteria with the THP-1 cells and before the lysing step. A killing step can comprise adding an antibiotic effective against the bacteria to the THP-1 cells, thereby killing bacteria not taken up by the THP-1 cells (i.e., extracellular bacteria). The antibiotic can be added at a concentration effective for killing the bacteria. The killing step, if used, can be performed after incubation of the bacteria with the THP-1 cells and before the lysing step. The killing step can be performed after or before a washing step, or between two washing steps. In some embodiments, the antibiotic is added to the THP-1 cells and incubated for 15-75 min, 20-60 min, 30-50 min, or about 42-45 min. In some embodiments, the antibiotic is gentamicin.

In some embodiments, the lysing step (b) is performed immediately after the infection step (0 h post-infection), 0-10 h post-infection, 1 h post-infection, 2 h post-infection, 3 h post-infection, 4 h post-infection, 5 h post-infection, 6 h post-infection, 7 h post-infection, 8 h post-infection, 9 h post-infection, or 10 h post-infection. In some embodiments, the lysing step is performed immediately after the infecting step (p0), 1 h post-infection (p1), 3 h post-infection (p3), or 5 h post-infection (p5). If lysis is not performed immediately after the infection step, the THP-1 cells can be incubated in growth media until lysis. Intracellular growth of the bacteria can occur during the post-infection incubation. The lysing step can comprise collecting the THP-1 cells in water or similar solvent capable of lysing the THP-1 cells, but not the bacteria, to form a lysate, and plating the lysate on media capable of supporting growth of the bacteria and allowing counting the number of colony forming units (CFUs). In some embodiments, the lysate can be diluted. In some embodiments, one or more different dilutions of the lysate can be plated on the media.

In some embodiments, the counting step can comprise determining the number of CFUs from the lysate. In some embodiments, the number of CFUs in an inoculating media is determined. In some embodiments, the number of CFUs is determined after different post-infection lysis periods or a bacteria strain. In some embodiments, CFUs for a bacteria strain are determined for the inoculating media, immediately after the infection step, and at one or more times post-infection. In some embodiments, CFUs for a bacteria strain are determined, immediately after the infection step and at three hours post-infection. In some embodiments, the CFUs determined at one time and compared with the CFUs determined at another post-infection time. In some embodiments, uptake, or infectivity rate is calculated by comparing the CFUs of the inoculating media with the CFUs at 0 h post-infection. In some embodiments, intracellular growth rate is calculated by comparing the CFUs at 1-10 h post-infection with the CFUs at 0 h post-infection. In some embodiments, intracellular growth rate is calculated by comparing the CFUs at 1 h, 3 h, or 5 h post-infection with the CFUs determined as 0 h post-infection.

Such methods can further comprise comparing uptake and/or intracellular growth of a test bacteria strain, such as a mutant, recombinant, or attenuated L. monocytogenes strain with a control, such as wild type Listeria strain, and/or a reference sample.

Additional embodiments are disclosed in the examples.

### III. Recombinant Bacteria or Listeria Strains

The methods disclosed herein assess attenuation and infectivity of bacteria strains, such as a Listeria strain. Such bacteria strains can be recombinant bacteria strains. Such recombinant bacteria strains can comprise a recombinant fusion polypeptide disclosed herein or a nucleic acid encoding the recombinant fusion polypeptide as disclosed elsewhere herein. Preferably, the bacteria strain is a Listeria strain, such as a Listeria monocytogenes (Lm) strain. Lm has a number of inherent advantages as a vaccine vector. The bacterium grows very efficiently in vitro without special requirements, and it lacks LPS, which is a major toxicity factor in gram-negative bacteria, such as Salmonella. Genetically attenuated Lm vectors also offer additional safety as they can be readily eliminated with antibiotics, in case of serious adverse effects, and unlike some viral vectors, no integration of genetic material into the host genome occurs.

The recombinant Listeria strain can be any Listeria strain. Examples of suitable Listeria strains include Listeria seeligeri, Listeria grayi, Listeria ivanovii, Listeria murrayi, Listeria welshimeri, Listeria monocytogenes (Lm), or any other known Listeria species. Preferably, the recombinant listeria strain is a strain of the species Listeria monocytogenes. Examples of Listeria monocytogenes strains include the following: L. monocytogenes 10403S wild type (see, e.g., Bishop and Hinrichs (1987) J Immunol 139:2005-2009; Lauer et al. (2002) J Bact 184:4177-4186); L. monocytogenes DP-L4056, which is phage cured (see, e.g., Lauer et al. (2002) J Bact 184:4177-4186); L. monocytogenes DP-L4027, which is phage cured and has an hly gene deletion (see, e.g., Lauer et al. (2002) J Bact 184:4177-4186; Jones and Portnoy (1994) Infect Immunity 65:5608-5613); L. monocytogenes DP-L4029, which is phage cured and has an actA gene deletion (see, e.g., Lauer et al. (2002) J Bact 184:4177-4186; Skoble et al. (2000) J Cell Biol 150:527-538); L. monocytogenes DP-L4042 (delta PEST) (see, e.g., Brockstedt et al. (2004) Proc Natl Acad Sci. USA 101:13832-13837 and supporting information); L. monocytogenes DP-L4097 (LLO-S44A) (see, e.g., Brockstedt et al. (2004) Proc Natl Acad Sci USA 101:13832-13837 and supporting information); L. monocytogenes DP-L4364 (delta lplA; lipoate protein ligase) (see, e.g., Brockstedt et al. (2004) Proc Natl Acad Sci USA 101:13832-13837 and supporting information); L. monocytogenes DP-L4405 (delta inlA) (see, e.g., Brockstedt et al. (2004) Proc Natl Acad Sci USA 101:13832-13837 and supporting information); L. monocytogenes DP-L4406 (delta inlB) (see, e.g., Brockstedt et al. (2004) Proc Natl Acad Sci USA 101:13832-13837 and supporting information); L. monocytogenes CS-LOOOl (delta actA; delta inlB) (see, e.g., Brockstedt et al. (2004) Proc Natl Acad Sci USA 101:13832-13837 and supporting information); L. monocytogenes CS-L0002 (delta actA; delta lplA) (see, e.g., Brockstedt et al. (2004) Proc Natl Acad Sci USA 101:13832-13837 and supporting information); L. monocytogenes CS-L0003 (LLO L461T; delta lplA) (see, e.g., Brockstedt et al. (2004) Proc Natl Acad Sci USA 101:13832-13837 and supporting information); L. monocytogenes DP-L4038 (delta actA; LLO L461T) (see, e.g., Brockstedt et al. (2004) Proc Nat Acad Sci USA 101:13832-13837 and supporting information); L. monocytogenes DP-L4384 (LLO S44A; LLO L461T) (see, e.g., Brockstedt et al. (2004) Proc Natl Acad Sci USA 101:13832-13837 and supporting information); a L. monocytogenes strain with an lpLA1 deletion (encoding lipoate protein ligase LplA1) (see, e.g., O'Riordan et al. (2003) Science 302:462-464); L. monocytogenes DP-L4017 (10403S with LLO L461T) (see, e.g., U.S. Pat. No. 7,691,393); L. monocytogenes EGD (see, e.g., GenBank Accession No. AL591824). In another embodiment, the Listeria strain is L. monocytogenes EGD-e (see GenBank Accession No. NC_003210; ATCC Accession No. BAA-679); L. monocytogenes DP-L4029 (actA deletion, optionally in combination with uvrAB deletion (DP-L4029uvrAB) (see, e.g., U.S. Pat. No. 7,691,393); L. monocytogenes actA-/inlB—double mutant (see, e.g., ATCC Accession No. PTA-5562); L. monocytogenes lplA mutant or hly mutant (see, e.g., US 2004/0013690); L. monocytogenes dal/dat double mutant (see, e.g., US 2005/0048081). Other L. monocytogenes strains includes those that are modified (e.g., by a plasmid and/or by genomic integration) to contain a nucleic acid encoding one of, or any combination of, the following genes: hly (LLO; listeriolysin); iap (p60); inlA; inlB; inlC; dal (alanine racemase); dat (D-amino acid aminotransferase); plcA; plcB; actA; or any nucleic acid that mediates growth, spread, breakdown of a single walled vesicle, breakdown of a double walled vesicle, binding to a host cell, or uptake by a host cell. Each of the above references is herein incorporated by reference in its entirety for all purposes.

The recombinant bacteria or Listeria can have wild-type virulence, can have attenuated virulence, or can be a virulent. For example, a recombinant Listeria of can be sufficiently virulent to escape the phagosome or phagolysosome and enter the cytosol. Such Listeria strains can also be live-attenuated Listeria strains, which comprise at least one attenuating mutation, deletion, or inactivation as disclosed elsewhere herein. Preferably, the recombinant Listeria is an attenuated auxotrophic strain. An auxotrophic strain is one that is unable to synthesize a particular organic compound required for its growth. Examples of such strains are described in U.S. Pat. No. 8,114,414, herein incorporated by reference in its entirety for all purposes.

Preferably, the recombinant Listeria strain lacks antibiotic resistance genes. For example, such recombinant Listeria strains can comprise a plasmid that does not encode an antibiotic resistance gene. However, some recombinant Listeria strains provided herein comprise a plasmid comprising a nucleic acid encoding an antibiotic resistance gene. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin.

A. Bacteria or Listeria Strains Comprising Recombinant Fusion Polypeptides or Nucleic Acids Encoding Recombinant Fusion Polypeptides

The recombinant bacteria strains (e.g., Listeria strains) disclosed herein comprise a recombinant fusion polypeptide disclosed herein or a nucleic acid encoding the recombinant fusion polypeptide as disclosed elsewhere herein.

In bacteria or Listeria strains comprising a nucleic acid encoding a recombinant fusion protein, the nucleic acid can be codon optimized. Examples of optimal codons utilized by L. monocytogenes for each amino acid are shown US 2007/0207170, herein incorporated by reference in its entirety for all purposes. A nucleic acid is codon-optimized if at least one codon in the nucleic acid is replaced with a codon that is more frequently used by L. monocytogenes for that amino acid than the codon in the original sequence.

The nucleic acid can be present in an episomal plasmid within the bacteria or Listeria strain and/or the nucleic acid can be genomically integrated in the bacteria or Listeria strain. Some recombinant bacteria or Listeria strains comprise two separate nucleic acids encoding two recombinant fusion polypeptides as disclosed herein: one nucleic acid in an episomal plasmid, and one genomically integrated in the bacteria or Listeria strain.

The episomal plasmid can be one that is stably maintained in vitro (in cell culture), in vivo (in a host), or both in vitro and in vivo. If in an episomal plasmid, the open reading frame encoding the recombinant fusion polypeptide can be operably linked to a promoter/regulatory sequence in the plasmid. If genomically integrated in the bacteria or Listeria strain, the open reading frame encoding the recombinant fusion polypeptide can be operably linked to an exogenous promoter/regulatory sequence or to an endogenous promoter/regulatory sequence. Examples of promoters/regulatory sequences useful for driving constitutive expression of a gene are well-known and include, for example, an hly, hlyA, actA, prfA, and p60 promoters of Listeria, the Streptococcus bac promoter, the Streptomyces griseus sgiA promoter, and the B. thuringiensis phaZ promoter. In some cases, an inserted gene of interest is not interrupted or subjected to regulatory constraints which often occur from integration into genomic DNA, and in some cases, the presence of the inserted heterologous gene does not lead to rearrangement or interruption of the cell's own important regions.

Such recombinant bacteria or Listeria strains can be made by transforming a bacteria or Listeria strain or an attenuated bacteria or Listeria strain described elsewhere herein with a plasmid or vector comprising a nucleic acid encoding the recombinant fusion polypeptide. The plasmid can be an episomal plasmid that does not integrate into a host chromosome. Alternatively, the plasmid can be an integrative plasmid that integrates into a chromosome of the bacteria or Listeria strain. The plasmids used herein can also be multicopy plasmids. Methods for transforming bacteria are well-known, and include calcium-chloride competent cell-based methods, electroporation methods, bacteriophage-mediated transduction, chemical transformation techniques, and physical transformation techniques. See, e.g., de Boer et al. (1989) Cell 56:641-649; Miller et al. (1995) FASEB J. 9:190-199; Sambrook et al. (1989) Molecular Cloning: A Laboratory Manual, Cold Spring Harbor Laboratory, New York; Ausubel et al. (1997) Current Protocols in Molecular Biology, John Wiley & Sons, New York; Gerhardt et al., eds., 1994, Methods for General and Molecular Bacteriology, American Society for Microbiology, Washington, D.C.; and Miller, 1992, A Short Course in Bacterial Genetics, Cold Spring Harbor Laboratory Press, Cold Spring Harbor, N.Y., each of which is herein incorporated by reference in its entirety for all purposes.

Bacteria or Listeria strains with genomically integrated heterologous nucleic acids can be made, for example, by using a site-specific integration vector, whereby the bacteria or Listeria comprising the integrated gene is created using homologous recombination. The integration vector can be any site-specific integration vector that is capable of infecting a bacteria or Listeria strain. Such an integration vector can comprise, for example, a PSA attPP′ site, a gene encoding a PSA integrase, a U153 attPP′ site, a gene encoding a U153 integrase, an A118 attPP′ site, a gene encoding an A118 integrase, or any other known attPP′ site or any other phage integrase.

Such bacteria or Listeria strains comprising an integrated gene can also be created using any other known method for integrating a heterologous nucleic acid into a bacteria or Listeria chromosome. Techniques for homologous recombination are well-known, and are described, for example, in Baloglu et al. (2005) Vet Microbiol 109(1-2):11-17); Jiang et al. 2005) Acta Biochim Biophys Sin (Shanghai) 37(1):19-24), and U.S. Pat. No. 6,855,320, each of which is herein incorporated by reference in its entirety for all purposes.

Integration into a bacteria or Listerial chromosome can also be achieved using transposon insertion. Techniques for transposon insertion are well-known, and are described, for example, for the construction of DP-L967 by Sun et al. (1990) Infection and Immunity 58: 3770-3778, herein incorporated by reference in its entirety for all purposes. Transposon mutagenesis can achieve stable genomic insertion, but the position in the genome where the heterologous nucleic acids has been inserted is unknown.

Integration into a bacterial or Listerial chromosome can also be achieved using phage integration sites (see, e.g., Lauer et al. (2002) J Bacteriol 184(15):4177-4186, herein incorporated by reference in its entirety for all purposes). For example, an integrase gene and attachment site of a bacteriophage (e.g., U153 or PSA listeriophage) can be used to insert a heterologous gene into the corresponding attachment site, which may be any appropriate site in the genome (e.g. comK or the 3′ end of the arg tRNA gene). Endogenous prophages can be cured from the utilized attachment site prior to integration of the heterologous nucleic acid. Such methods can result, for example, in single-copy integrants. In order to avoid a “phage curing step,” a phage integration system based on PSA phage can be used (see, e.g., Lauer et al. (2002) J Bacteriol 184:4177-4186, herein incorporated by reference in its entirety for all purposes). Maintaining the integrated gene can require, for example, continuous selection by antibiotics. Alternatively, a phage-based chromosomal integration system can be established that does not require selection with antibiotics. Instead, an auxotrophic host strain can be complemented. For example, a phage-based chromosomal integration system for clinical applications can be used, where a host strain that is auxotrophic for essential enzymes, including, for example, D-alanine racemase is used (e.g., Lm dal(−)dat(−)).

Conjugation can also be used to introduce genetic material and/or plasmids into bacteria. Methods for conjugation are well-known, and are described, for example, in Nikodinovic et al. (2006) Plasmid 56(3):223-227 and Auchtung et al. (2005) Proc Nat Acad Sci USA 102(35):12554-12559, each of which is herein incorporated by reference in its entirety for all purposes.

In a specific example, a recombinant bacteria or Listeria strain can comprise a nucleic acid encoding a recombinant fusion polypeptide genomically integrated into the bacteria or Listeria genome as an open reading frame with an endogenous actA sequence (encoding an ActA protein) or an endogenous hly sequence (encoding an LLO protein). For example, the expression and secretion of the fusion polypeptide can be under the control of the endogenous actA promoter and ActA signal sequence or can be under the control of the endogenous hly promoter and LLO signal sequence. As another example, the nucleic acid encoding a recombinant fusion polypeptide can replace an actA sequence encoding an ActA protein or an hly sequence encoding an LLO protein.

Selection of recombinant bacteria or Listeria strains can be achieved by any means. For example, antibiotic selection can be used. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin. Alternatively, auxotrophic strains can be used, and an exogenous metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. Alternatively, a temperature-sensitive plasmid can be used to select recombinants or any other known means for selecting recombinants.

B. Attenuation of Bacteria or Listeria Strains

The recombinant bacteria strains (e.g., recombinant Listeria strains) disclosed herein can be attenuated. The term “attenuation” encompasses a diminution in the ability of the bacterium to cause disease in a host animal. For example, the pathogenic characteristics of an attenuated Listeria strain may be lessened compared with wild-type Listeria, although the attenuated Listeria is capable of growth and maintenance in culture. Using as an example the intravenous inoculation of BALB/c mice with an attenuated Listeria, in some embodiments, the lethal dose at which 50% of inoculated animals survive (LD₅₀) is increased above the LD₅₀ of wild-type Listeria by at least about 10-fold, at least about 100-fold, at least about 1,000 fold, at least about 10,000 fold, or at least about 100,000-fold. An attenuated strain of Listeria is thus one that does not kill an animal to which it is administered, or is one that kills the animal only when the number of bacteria administered is vastly greater than the number of wild-type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. Attenuated strains are environmentally safe in that they are incapable of uncontrolled replication

(1) Methods of Attenuating Bacteria and Listeria Strains

Attenuation can be accomplished by any known means. For example, such attenuated strains can be deficient in one or more endogenous virulence genes or one or more endogenous metabolic genes. Examples of such genes are disclosed herein, and attenuation can be achieved by inactivation of any one of or any combination of the genes disclosed herein. Inactivation can be achieved, for example, through deletion or through mutation (e.g., an inactivating mutation). The term “mutation” includes any type of mutation or modification to the sequence (nucleic acid or amino acid sequence) and may encompass a deletion, a truncation, an insertion, a substitution, a disruption, or a translocation. For example, a mutation can include a frameshift mutation, a mutation which causes premature termination of a protein, or a mutation of regulatory sequences which affect gene expression. Mutagenesis can be accomplished using recombinant DNA techniques or using traditional mutagenesis technology using mutagenic chemicals or radiation and subsequent selection of mutants. Deletion mutants may be preferred because of the accompanying low probability of reversion. The term “metabolic gene” refers to a gene encoding an enzyme involved in or required for synthesis of a nutrient utilized or required by a host bacteria. For example, the enzyme can be involved in or required for the synthesis of a nutrient required for sustained growth of the host bacteria. The term “virulence” gene includes a gene whose presence or activity in an organism's genome that contributes to the pathogenicity of the organism (e.g., enabling the organism to achieve colonization of a niche in the host (including attachment to cells), immunoevasion (evasion of host's immune response), immunosuppression (inhibition of host's immune response), entry into and exit out of cells, or obtaining nutrition from the host).

A specific example of such an attenuated strain is Listeria monocytogenes (Lm) dal(−)dat(−) (Lmdd). Another example of such an attenuated strain is Lm dal(−)dat(−)AactA (LmddA). See, e.g., US 2011/0142791, herein incorporated by references in its entirety for all purposes. LmddA is based on a Listeria strain which is attenuated due to the deletion of the endogenous virulence gene actA. Such strains can retain a plasmid for antigen expression in vivo and in vitro by complementation of the dal gene. Alternatively, the LmddA can be a dal/dat/actA Listeria having mutations in the endogenous dal, dat, and actA genes. Such mutations can be, for example, a deletion or other inactivating mutation.

Another specific example of an attenuated strain is LmprfA(−) or a strain having a partial deletion or inactivating mutation in the prfA gene. The PrfA protein controls the expression of a regulon comprising essential virulence genes required by Lm to colonize its vertebrate hosts; hence the prfA mutation strongly impairs PrfA ability to activate expression of PrfA-dependent virulence genes.

Yet another specific example of an attenuated strain is Lm inlB(−)actA(−) in which two genes critical to the bacterium's natural virulence—internalin B and act A—are deleted.

Other examples of attenuated bacteria or Listeria strains include bacteria or Listeria strains deficient in one or more endogenous virulence genes. Examples of such genes include actA, prfA, plcB, plcA, inlA, inlB, inlC, inU, and bsh in Listeria. Attenuated Listeria strains can also be the double mutant or triple mutant of any of the above-mentioned strains. Attenuated Listeria strains can comprise a mutation or deletion of each one of the genes, or comprise a mutation or deletion of, for example, up to ten of any of the genes provided herein (e.g., including the actA, prfA, and dal/dat genes). For example, an attenuated Listeria strain can comprise a mutation or deletion of an endogenous internalin C(inlC) gene and/or a mutation or deletion of an endogenous actA gene. Alternatively, an attenuated Listeria strain can comprise a mutation or deletion of an endogenous internalin B (inlB) gene and/or a mutation or deletion of an endogenous actA gene. Alternatively, an attenuated Listeria strain can comprise a mutation or deletion of endogenous inlB, inlC, and actA genes. Translocation of Listeria to adjacent cells is inhibited by the deletion of the endogenous actA gene and/or the endogenous inlC gene or endogenous inlB gene, which are involved in the process, thereby resulting in high levels of attenuation with increased immunogenicity and utility as a strain backbone. An attenuated Listeria strain can also be a double mutant comprising mutations or deletions of both plcA and plcB. In some cases, the strain can be constructed from the EGD Listeria backbone.

A bacteria or Listeria strain can also be an auxotrophic strain having a mutation in a metabolic gene. As one example, the strain can be deficient in one or more endogenous amino acid metabolism genes. For example, the generation of auxotrophic strains of Listeria deficient in D-alanine, for example, may be accomplished in a number of ways that are well-known, including deletion mutations, insertion mutations, frameshift mutations, mutations which cause premature termination of a protein, or mutation of regulatory sequences which affect gene expression. Deletion mutants may be preferred because of the accompanying low probability of reversion of the auxotrophic phenotype. As an example, mutants of D-alanine which are generated according to the protocols presented herein may be tested for the ability to grow in the absence of D-alanine in a simple laboratory culture assay. Those mutants which are unable to grow in the absence of this compound can be selected.

Examples of endogenous amino acid metabolism genes include a vitamin synthesis gene, a gene encoding pantothenic acid synthase, a D-glutamic acid synthase gene, a D-alanine amino transferase (dat) gene, a D-alanine racemase (dal) gene, dga, a gene involved in the synthesis of diaminopimelic acid (DAP), a gene involved in the synthesis of Cysteine synthase A (cysK), a vitamin-B12 independent methionine synthase, trpA, trpB, trpE, asnB, gltD, gltB, leuA, argG, and thrC. The Listeria strain can be deficient in two or more such genes (e.g., dat and dal). D-glutamic acid synthesis is controlled in part by the dal gene, which is involved in the conversion of D-glu+pyr to alpha-ketoglutarate+D-ala, and the reverse reaction.

As another example, an attenuated Listeria strain can be deficient in an endogenous synthase gene, such as an amino acid synthesis gene. Examples of such genes include folP, a gene encoding a dihydrouridine synthase family protein, ispD, ispF, a gene encoding a phosphoenolpyruvate synthase, hisF, hisH, fli, a gene encoding a ribosomal large subunit pseudouridine synthase, ispD, a gene encoding a bifunctional GMP synthase/glutamine amidotransferase protein, cobS, cobB, cbiD, a gene encoding a uroporphyrin-III C-methyltransferase/uroporphyrinogen-III synthase, cobQ, uppS, truB, dxs, mvaS, dapA, ispG, folC, a gene encoding a citrate synthase, argJ, a gene encoding a 3-deoxy-7-phosphoheptulonate synthase, a gene encoding an indole-3-glycerol-phosphate synthase, a gene encoding an anthranilate synthase/glutamine amidotransferase component, menB, a gene encoding a menaquinone-specific isochorismate synthase, a gene encoding a phosphoribosylformylglycinamidine synthase I or II, a gene encoding a phosphoribosylaminoimidazole-succinocarboxamide synthase, carB, carA, thyA, mgsA, aroB, hepB, rluB, ilvB, ilvN, alsS, fabF, fabH, a gene encoding a pseudouridine synthase, pyrG, truA, pabB, and an atp synthase gene (e.g., atpC, atpD-2, aptG, atpA-2, and so forth).

Attenuated Listeria strains can be deficient in endogenousphoP, aroA, aroC, aroD, or plcB. As yet another example, an attenuated Listeria strain can be deficient in an endogenous peptide transporter. Examples include genes encoding an ABC transporter/ATP-binding/permease protein, an oligopeptide ABC transporter/oligopeptide-binding protein, an oligopeptide ABC transporter/permease protein, a zinc ABC transporter/zinc-binding protein, a sugar ABC transporter, a phosphate transporter, a ZIP zinc transporter, a drug resistance transporter of the EmrB/QacA family, a sulfate transporter, a proton-dependent oligopeptide transporter, a magnesium transporter, a formate/nitrite transporter, a spermidine/putrescine ABC transporter, a Na/Pi-cotransporter, a sugar phosphate transporter, a glutamine ABC transporter, a major facilitator family transporter, a glycine betaine/L-proline ABC transporter, a molybdenum ABC transporter, a techoic acid ABC transporter, a cobalt ABC transporter, an ammonium transporter, an amino acid ABC transporter, a cell division ABC transporter, a manganese ABC transporter, an iron compound ABC transporter, a maltose/maltodextrin ABC transporter, a drug resistance transporter of the Bcr/CflA family, and a subunit of one of the above proteins.

Other attenuated bacteria and Listeria strains can be deficient in an endogenous metabolic enzyme that metabolizes an amino acid that is used for a bacterial growth process, a replication process, cell wall synthesis, protein synthesis, metabolism of a fatty acid, or for any other growth or replication process. Likewise, an attenuated strain can be deficient in an endogenous metabolic enzyme that can catalyze the formation of an amino acid used in cell wall synthesis, can catalyze the synthesis of an amino acid used in cell wall synthesis, or can be involved in synthesis of an amino acid used in cell wall synthesis. Alternatively, the amino acid can be used in cell wall biogenesis. Alternatively, the metabolic enzyme is a synthetic enzyme for D-glutamic acid, a cell wall component.

Other attenuated Listeria strains can be deficient in metabolic enzymes encoded by a D-glutamic acid synthesis gene, dga, an alr (alanine racemase) gene, or any other enzymes that are involved in alanine synthesis. Yet other examples of metabolic enzymes for which the Listeria strain can be deficient include enzymes encoded by serC (a phosphoserine aminotransferase), asd (aspartate betasemialdehyde dehydrogenase; involved in synthesis of the cell wall constituent diaminopimelic acid), the gene encoding gsaB-glutamate-1-semialdehyde aminotransferase (catalyzes the formation of 5-aminolevulinate from (S)-4-amino-5-oxopentanoate), hemL (catalyzes the formation of 5-aminolevulinate from (S)-4-amino-5-oxopentanoate), aspB (an aspartate aminotransferase that catalyzes the formation of oxalozcetate and L-glutamate from L-aspartate and 2-oxoglutarate), argF-1 (involved in arginine biosynthesis), aroE (involved in amino acid biosynthesis), aroB (involved in 3-dehydroquinate biosynthesis), aroD (involved in amino acid biosynthesis), aroC (involved in amino acid biosynthesis), hisB (involved in histidine biosynthesis), hisD (involved in histidine biosynthesis), hisG (involved in histidine biosynthesis), metX (involved in methionine biosynthesis), proB (involved in proline biosynthesis), argR (involved in arginine biosynthesis), argJ (involved in arginine biosynthesis), thil (involved in thiamine biosynthesis), LMOf2365_1652 (involved in tryptophan biosynthesis), aroA (involved in tryptophan biosynthesis), ilvD (involved in valine and isoleucine biosynthesis), ilvC (involved in valine and isoleucine biosynthesis), leuA (involved in leucine biosynthesis), dapF (involved in lysine biosynthesis), and thrB (involved in threonine biosynthesis) (all GenBank Accession No. NC_002973).

An attenuated Listeria strain can be generated by mutation of other metabolic enzymes, such as a tRNA synthetase. For example, the metabolic enzyme can be encoded by the trpS gene, encoding tryptophanyltRNA synthetase. For example, the host strain bacteria can be Δ(trpS aroA), and both markers can be contained in an integration vector.

Other examples of metabolic enzymes that can be mutated to generate an attenuated Listeria strain include an enzyme encoded by murE (involved in synthesis of diaminopimelic acid; GenBank Accession No: NC_003485), LMOf2365_2494 (involved in teichoic acid biosynthesis), WecE (Lipopolysaccharide biosynthesis protein rffA; GenBank Accession No: AE014075.1), or amiA (an N-acetylmuramoyl-L-alanine amidase). Yet other examples of metabolic enzymes include aspartate aminotransferase, histidinol-phosphate aminotransferase (GenBank Accession No. NP_466347), or the cell wall teichoic acid glycosylation protein GtcA.

Other examples of metabolic enzymes that can be mutated to generate an attenuated Listeria strain include a synthetic enzyme for a peptidoglycan component or precursor. The component can be, for example, UDP-N-acetylmuramylpentapeptide, UDP-N-acetylglucosamine, MurNAc-(pentapeptide)-pyrophosphoryl-undecaprenol, GlcNAc-p-(1,4)-MurNAc-(pentapeptide)-pyrophosphorylundecaprenol, or any other peptidoglycan component or precursor.

Yet other examples of metabolic enzymes that can be mutated to generate an attenuated Listeria strain include metabolic enzymes encoded by murG, murD, murA-1, or murA-2 (all set forth in GenBank Accession No. NC_002973). Alternatively, the metabolic enzyme can be any other synthetic enzyme for a peptidoglycan component or precursor. The metabolic enzyme can also be a trans-glycosylase, a trans-peptidase, a carboxy-peptidase, any other class of metabolic enzyme, or any other metabolic enzyme. For example, the metabolic enzyme can be any other Listeria metabolic enzyme or any other Listeria monocytogenes metabolic enzyme.

Other bacteria strains can be attenuated as described above for Listeria by mutating the corresponding orthologous genes in the other bacteria strains.

(2) Methods of Complementing Attenuated Bacteria and Listeria Strains

The attenuated bacteria or Listeria strains disclosed herein can further comprise a nucleic acid comprising a complementing gene or encoding a metabolic enzyme that complements an attenuating mutation (e.g., complements the auxotrophy of the auxotrophic Listeria strain). For example, a nucleic acid having a first open reading frame encoding a fusion polypeptide as disclosed herein can further comprise a second open reading frame comprising the complementing gene or encoding the complementing metabolic enzyme. Alternatively, a first nucleic acid can encode the fusion polypeptide and a separate second nucleic acid can comprise the complementing gene or encode the complementing metabolic enzyme.

The complementing gene can be extrachromosomal or can be integrated into the bacteria or Listeria genome. For example, the auxotrophic Listeria strain can comprise an episomal plasmid comprising a nucleic acid encoding a metabolic enzyme. Such plasmids will be contained in the Listeria in an episomal or extrachromosomal fashion. Alternatively, the auxotrophic Listeria strain can comprise an integrative plasmid (i.e., integration vector) comprising a nucleic acid encoding a metabolic enzyme. Such integrative plasmids can be used for integration into a Listeria chromosome. Preferably, the episomal plasmid or the integrative plasmid lacks an antibiotic resistance marker.

The metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. For example, a bacteria auxotrophic for D-glutamic acid synthesis can be transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. Similarly, a bacterium auxotrophic for D-alanine synthesis will grow in the absence of D-alanine when transformed and expressing a plasmid comprising a nucleic acid encoding an amino acid metabolism enzyme for D-alanine synthesis. Such methods for making appropriate media comprising or lacking necessary growth factors, supplements, amino acids, vitamins, antibiotics, and the like are well-known and are available commercially.

Once the auxotrophic bacteria comprising the plasmid encoding a metabolic enzyme or a complementing gene provided herein have been selected in appropriate medium, the bacteria can be propagated in the presence of a selective pressure. Such propagation can comprise growing the bacteria in media without the auxotrophic factor. The presence of the plasmid expressing the metabolic enzyme or the complementing gene in the auxotrophic bacteria ensures that the plasmid will replicate along with the bacteria, thus continually selecting for bacteria harboring the plasmid. Production of the bacteria or Listeria strain can be readily scaled up by adjusting the volume of the medium in which the auxotrophic bacteria comprising the plasmid are growing.

In one specific example, the attenuated strain is a strain having a deletion of or an inactivating mutation in dal and dat (e.g., Listeria monocytogenes (Lm) dal(−)dat(−) (Lmdd) or Lm dal(−)dat(−)AactA (LmddA)), and the complementing gene encodes an alanine racemase enzyme (e.g., encoded by dal gene) or a D-amino acid aminotransferase enzyme (e.g., encoded by dat gene). An exemplary alanine racemase protein can have the sequence set forth in SEQ ID NO: 76 (encoded by SEQ ID NO: 78; GenBank Accession No: AF038438) or can be a homologue, variant, isoform, analog, fragment, fragment of a homologue, fragment of a variant, fragment of an analog, or fragment of an isoform of SEQ ID NO: 76. The alanine racemase protein can also be any other Listeria alanine racemase protein. Alternatively, the alanine racemase protein can be any other gram-positive alanine racemase protein or any other alanine racemase protein. An exemplary D-amino acid aminotransferase protein can have the sequence set forth in SEQ ID NO: 77 (encoded by SEQ ID NO: 79; GenBank Accession No: AF038439) or can be a homologue, variant, isoform, analog, fragment, fragment of a homologue, fragment of a variant, fragment of an analog, or fragment of an isoform of SEQ ID NO: 77. The D-amino acid aminotransferase protein can also be any other Listeria D-amino acid aminotransferase protein. Alternatively, the D-amino acid aminotransferase protein can be any other gram-positive D-amino acid aminotransferase protein or any other D-amino acid aminotransferase protein.

In another specific example, the attenuated strain is a strain having a deletion of or an inactivating mutation in prfA (e.g., Lm prfA(−)), and the complementing gene encodes a PrfA protein. For example, the complementing gene can encode a mutant PrfA (D133V) protein that restores partial PrfA function. An example of a wild type PrfA protein is set forth in SEQ ID NO: 80 (encoded by nucleic acid set forth in SEQ ID NO: 81), and an example of a D133V mutant PrfA protein is set forth in SEQ ID NO: 82 (encoded by nucleic acid set forth in SEQ ID NO: 83). The complementing PrfA protein can be a homologue, variant, isoform, analog, fragment, fragment of a homologue, fragment of a variant, fragment of an analog, or fragment of an isoform of SEQ ID NO: 80 or 82. The PrfA protein can also be any other Listeria PrfA protein. Alternatively, the PrfA protein can be any other gram-positive PrfA protein or any other PrfA protein.

In another example, the bacteria strain or Listeria strain can comprise a deletion of or an inactivating mutation in an actA gene, and the complementing gene can comprise an actA gene to complement the mutation and restore function to the Listeria strain.

Other auxotroph strains and complementation systems can also be adopted for the use with the methods and compositions provided herein.

### IV. Recombinant Fusion Polypeptides

The recombinant fusion polypeptides in the recombinant bacteria or Listeria strains disclosed herein can be in any form. Some such fusion polypeptides can comprise a PEST-containing peptide fused to one or more disease-associated antigenic peptides. Other such recombinant fusion polypeptides can comprise one or more disease-associated antigenic peptides, and wherein the fusion polypeptide does not comprise a PEST-containing peptide.

Another example of a recombinant fusion polypeptides comprises from N-terminal end to C-terminal end a bacterial secretion sequence, a ubiquitin (Ub) protein, and one or more disease-associated antigenic peptides (i.e., in tandem, such as Ub-peptide1-peptide2). Alternatively, if two or more disease-associated antigenic peptides are used, a combination of separate fusion polypeptides can be used in which each antigenic peptide is fused to its own secretion sequence and Ub protein (e.g., Ub-peptide1; Ub2-peptide2).

Nucleic acids (termed minigene constructs) encoding such recombinant fusion polypeptides are also disclosed. Such minigene nucleic acid constructs can further comprise two or more open reading frames linked by a Shine-Dalgarno ribosome binding site nucleic acid sequence between each open reading frame. For example, a minigene nucleic acid construct can further comprise two to four open reading frames linked by a Shine-Dalgarno ribosome binding site nucleic acid sequence between each open reading frame. Each open reading frame can encode a different polypeptide. In some nucleic acid constructs, the codon encoding the carboxy terminus of the fusion polypeptide is followed by two stop codons to ensure termination of protein synthesis.

The bacterial signal sequence can be a Listerial signal sequence, such as an Hly or an ActA signal sequence, or any other known signal sequence. In other cases, the signal sequence can be an LLO signal sequence. An exemplary LLO signal sequence is set forth in SEQ ID NO: 97. The signal sequence can be bacterial, can be native to a host bacterium (e.g., Listeria monocytogenes, such as a secA1 signal peptide), or can be foreign to a host bacterium. Specific examples of signal peptides include an Usp45 signal peptide from Lactococcus lactis, a Protective Antigen signal peptide from Bacillus anthracis, a secA2 signal peptide such the p60 signal peptide from Listeria monocytogenes, and a Tat signal peptide such as a B. subtilis Tat signal peptide (e.g., PhoD). In specific examples, the secretion signal sequence is from a Listeria protein, such as an ActA₃₀₀ secretion signal or an ActA₁₀₀ secretion signal. An exemplary ActA signal sequence is set forth in SEQ ID NO: 98.

The ubiquitin can be, for example, a full-length protein. The ubiquitin expressed from the nucleic acid construct provided herein can be cleaved at the carboxy terminus from the rest of the recombinant fusion polypeptide expressed from the nucleic acid construct through the action of hydrolases upon entry to the host cell cytosol. This liberates the amino terminus of the fusion polypeptide, producing a peptide in the host cell cytosol.

Selection of, variations of, and arrangement of antigenic peptides within a fusion polypeptide are discussed in detail elsewhere herein, and examples of disease-associated antigenic peptides are discussed in more detail elsewhere herein.

The recombinant fusion polypeptides can comprise one or more tags. For example, the recombinant fusion polypeptides can comprise one or more peptide tags N-terminal and/or C-terminal to one or more antigenic peptides. A tag can be fused directly to an antigenic peptide or linked to an antigenic peptide via a linker (examples of which are disclosed elsewhere herein). Examples of tags include the following: FLAG tag; 2×FLAG tag; 3×FLAG tag; His tag, 6×His tag; and SIINFEKL tag. An exemplary SIINFEKL tag is set forth in SEQ ID NO: 16 (encoded by any one of the nucleic acids set forth in SEQ ID NOS: 1-15). An exemplary 3×FLAG tag is set forth in SEQ ID NO: 32 (encoded by anyone of the nucleic acids set forth in SEQ ID NOS: 17-31). An exemplary variant 3×FLAG tag is set forth in SEQ ID NO: 99. Two or more tags can be used together, such as a 2×FLAG tag and a SIINFEKL tag, a 3×FLAG tag and a SIINFEKL tag, or a 6×His tag and a SIINFEKL tag. If two or more tags are used, they can be located anywhere within the recombinant fusion polypeptide and in any order. For example, the two tags can be at the C-terminus of the recombinant fusion polypeptide, the two tags can be at the N-terminus of the recombinant fusion polypeptide, the two tags can be located internally within the recombinant fusion polypeptide, one tag can be at the C-terminus and one tag at the N-terminus of the recombinant fusion polypeptide, one tag can be at the C-terminus and one internally within the recombinant fusion polypeptide, or one tag can be at the N-terminus and one internally within the recombinant fusion polypeptide. Other tags include chitin binding protein (CBP), maltose binding protein (MBP), glutathione-S-transferase (GST), thioredoxin (TRX), and poly(NANP). Particular recombinant fusion polypeptides comprise a C-terminal SIINFEKL tag. Such tags can allow for easy detection of the recombinant fusion protein, confirmation of secretion of the recombinant fusion protein, or for following the immunogenicity of the secreted fusion polypeptide by following immune responses to these “tag” sequence peptides. Such immune response can be monitored using a number of reagents including, for example, monoclonal antibodies and DNA or RNA probes specific for these tags.

The recombinant fusion polypeptides disclosed herein can be expressed by recombinant Listeria strains or can be expressed and isolated from other vectors and cell systems used for protein expression and isolation. Recombinant Listeria strains comprising expressing such antigenic peptides can be used, for example in immunogenic compositions comprising such recombinant Listeria and in vaccines comprising the recombinant Listeria strain and an adjuvant. Expression of one or more antigenic peptides as a fusion polypeptides with a nonhemolytic truncated form of LLO, ActA, or a PEST-like sequence in host cell systems in Listeria strains and host cell systems other than Listeria can result in enhanced immunogenicity of the antigenic peptides.

Nucleic acids encoding such recombinant fusion polypeptides are also disclosed. The nucleic acid can be in any form. The nucleic acid can comprise or consist of DNA or RNA, and can be single-stranded or double-stranded. The nucleic acid can be in the form of a plasmid, such as an episomal plasmid, a multicopy episomal plasmid, or an integrative plasmid. Alternatively, the nucleic acid can be in the form of a viral vector, a phage vector, or in a bacterial artificial chromosome. Such nucleic acids can have one open reading frame or can have two or more open reading frames (e.g., an open reading frame encoding the recombinant fusion polypeptide and a second open reading frame encoding a metabolic enzyme). In one example, such nucleic acids can comprise two or more open reading frames linked by a Shine-Dalgarno ribosome binding site nucleic acid sequence between each open reading frame. For example, a nucleic acid can comprise two to four open reading frames linked by a Shine-Dalgarno ribosome binding site nucleic acid sequence between each open reading frame. Each open reading frame can encode a different polypeptide. In some nucleic acids, the codon encoding the carboxy terminus of the fusion polypeptide is followed by two stop codons to ensure termination of protein synthesis.

A. Antigenic Peptides

Disease-associated peptides include peptides from proteins that are expressed in a particular disease. For example, such peptides may be from proteins that are expressed in a disease tissue but not in a corresponding normal tissue, or that are expressed at abnormally high levels in a disease tissue. The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life. Examples of disease-associated antigenic peptides can include Human Papilloma Virus (HPV) E7 or E6, a Prostate Specific Antigen (PSA), a chimeric Her2 antigen, Her2/neu chimeric antigen. The Human Papilloma Virus can be HPV 16 or HPV 18. The antigenic peptide can also include HPV16 E6, HPV16 E7, HPV18 E6, HPV18 E7 antigens operably linked in tandem or HPV16 antigenic peptide operably linked in tandem to an HPV antigenic peptide.

The fusion polypeptide can include a single antigenic peptide or can includes two or more antigenic peptides. Each antigenic peptide can be of any length sufficient to induce an immune response, and each antigenic peptide can be the same length or the antigenic peptides can have different lengths. For example, an antigenic peptide disclosed herein can be 5-100, 15-50, or 21-27 amino acids in length, or 15-100, 15-95, 15-90, 15-85, 15-80, 15-75, 15-70, 15-65, 15-60, 15-55, 15-50, 15-45, 15-40, 15-35, 15-30, 20-100, 20-95, 20-90, 20-85, 20-80, 20-75, 20-70, 20-65, 20-60, 20-55, 20-50, 20-45, 20-40, 20-35, 20-30, 11-21, 15-21, 21-31, 31-41, 41-51, 51-61, 61-71, 71-81, 81-91, 91-101, 101-121, 121-141, 141-161, 161-181, 181-201, 8-27, 10-30, 10-40, 15-30, 15-40, 15-25, 1-10, 10-20, 20-30, 30-40, 1-100, 5-75, 5-50, 5-40, 5-30, 5-20, 5-15, 5-10, 1-75, 1-50, 1-40, 1-30, 1-20, 1-15, 1-10, 8-11, or 11-16 amino acids in length. For example, an antigenic peptide can be at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids in length. Some specific examples of antigenic peptides are 21 or 27 amino acids in length. Other antigenic peptides can be full-length proteins or fragments thereof.

As one example, an antigenic peptide can comprise a neoepitope. These neoepitopes can be, for example, patient-specific (i.e., subject-specific) cancer mutations. Antigenic peptides comprising neoepitopes can be generated in a process for creating a personalized immunotherapy comprising comparing nucleic acids extracted from a cancer sample from a subject to nucleic acids extracted from a normal or healthy reference sample in order to identify somatic mutations or sequence differences present in the cancer sample compared with the normal or healthy sample. For examples, these mutations or sequence differences can be somatic, nonsynonymous missense mutations, or somatic frameshift mutations, and can encode an expressed amino acid sequence. A peptide expressing such somatic mutations or sequence differences can be referred to as a “neoepitope.” A cancer-specific neoepitope may refer to an epitope that is not present in a reference sample (such as a normal non-cancerous or germline cell or tissue) but is found in a cancer sample. This includes, for example, situations in which in a normal non-cancerous or germline cell a corresponding epitope is found, but due to one or more mutations in a cancer cell, the sequence of the epitope is changed so as to result in the neoepitope. A neoepitope can comprise a mutated epitope, and can comprise non-mutated sequence on either or both sides of the mutation.

As another example, antigenic peptides can comprise recurrent cancer mutations. For example, a recombinant fusion polypeptide disclosed herein can comprise a PEST-containing peptide fused to two or more antigenic peptides (i.e., in tandem, such as PEST-peptide1-peptide2) or can comprise two or more antigenic peptides not fused to a PEST-containing peptide, wherein each antigenic peptide comprises a single, recurrent cancer mutation (i.e., a single, recurrent change in the amino acid sequence of a protein, or a sequence encoded by a single, different, nonsynonymous, recurrent cancer mutation in a gene), and wherein at least two of the antigenic peptides comprise different recurrent cancer mutations and are fragments of the same cancer-associated protein. Alternatively, each of the antigenic peptides can comprise a different recurrent cancer mutation from a different cancer-associated protein. Alternatively, a combination of separate fusion polypeptides can be used in which each antigenic peptide is fused (or is not fused) to its own PEST-containing peptide (e.g., PEST1-peptide1; PEST2-peptide2). Optionally, some or all of the fragments are non-contiguous fragments of the same cancer-associated protein. Non-contiguous fragments are fragments that do not occur sequentially in a protein sequence (e.g., the first fragment consists of residues 10-30, and the second fragment consists of residues 100-120; or the first fragment consists of residues 10-30, and the second fragment consists of residues 20-40). Optionally, each of the antigenic peptides comprises a different recurrent cancer mutation from a single type of cancer.

Recurrent cancer mutations can be from cancer-associated proteins. The term “cancer-associated protein” includes proteins having mutations that occur in multiple types of cancer, that occur in multiple subjects having a particular type of cancer, or that are correlated with the occurrence or progression of one or more types of cancer. For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). Preferably, a cancer-associated protein has a “mutational hotspot.” A mutational hotspot is an amino acid position in a protein-coding gene that is mutated (preferably by somatic substitutions rather than other somatic abnormalities, such as translocations, amplifications, and deletions) more frequently than would be expected in the absence of selection. Such hotspot mutations can occur across multiple types of cancer and/or can be shared among multiple cancer patients. Mutational hotspots indicate selective pressure across a population of tumor samples. Tumor genomes contain recurrent cancer mutations that “drive” tumorigenesis by affecting genes (i.e., tumor driver genes) that confer selective growth advantages to the tumor cells upon alteration. Such tumor driver genes can be identified, for example, by identifying genes that are mutated more frequently than expected from the background mutation rate (i.e., recurrence); by identifying genes that exhibit other signals of positive selection across tumor samples (e.g., a high rate of non-silent mutations compared to silent mutations, or a bias towards the accumulation of functional mutations); by exploiting the tendency to sustain mutations in certain regions of the protein sequence based on the knowledge that whereas inactivating mutations are distributed along the sequence of the protein, gain-of-function mutations tend to occur specifically in particular residues or domains; or by exploiting the overrepresentation of mutations in specific functional residues, such as phosphorylation sites. Many of these mutations frequently occur in the functional regions of biologically active proteins (for example, kinase domains or binding domains) or interrupt active sites (for example, phosphorylation sites) resulting in loss-of-function or gain-of-function mutations, or they can occur in such a way that the three-dimensional structure and/or charge balance of the protein is perturbed sufficiently to interfere with normal function. Genomic analysis of large numbers of tumors reveals that mutations often occur at a limited number of amino acid positions. Therefore, a majority of the common mutations can be represented by a relatively small number of potential tumor-associated antigens or T cell epitopes.

A “recurrent cancer mutation” is a change in the amino acid sequence of a protein that occurs in multiple types of cancer and/or in multiple subjects having a particular types of cancer. Such mutations associated with a cancer can result in tumor-associated antigens that are not normally present in corresponding healthy tissue.

Tumor-driver genes and cancer-associated proteins having common mutations that occur across multiple cancers or among multiple cancer patients are known, and sequencing data across multiple tumor samples and multiple tumor types exists. See, e.g., Chang et al. (2016) Nat Biotechnol 34(2):155-163; Tamborero et al. (2013) Sci Rep 3:2650, each of which is herein incorporated by reference in its entirety.

Each antigenic peptide can also be hydrophilic or can score up to or below a certain hydropathy threshold, which can be predictive of secretability in Listeria monocytogenes or another bacteria of interest. For example, antigenic peptides can be scored by a Kyte and Doolittle hydropathy index 21 amino acid window, and all scoring above a cutoff (around 1.6) can be excluded as they are unlikely to be secretable by Listeria monocytogenes. Likewise, the combination of antigenic peptides or the fusion polypeptide can be hydrophilic or can score up to or below a certain hydropathy threshold, which can be predictive of secretability in Listeria monocytogenes or another bacteria of interest.

The antigenic peptides can be linked together in any manner. For example, the antigenic peptides can be fused directly to each other with no intervening sequence. Alternatively, the antigenic peptides can be linked to each other indirectly via one or more linkers, such as peptide linkers. In some cases, some pairs of adjacent antigenic peptides can be fused directly to each other, and other pairs of antigenic peptides can be linked to each other indirectly via one or more linkers. The same linker can be used between each pair of adjacent antigenic peptides, or any number of different linkers can be used between different pairs of adjacent antigenic peptides. In addition, one linker can be used between a pair of adjacent antigenic peptides, or multiple linkers can be used between a pair of adjacent antigenic peptides.

Any suitable sequence can be used for a peptide linker. As an example, a linker sequence may be, for example, from 1 to about 50 amino acids in length. Some linkers may be hydrophilic. The linkers can serve varying purposes. For example, the linkers can serve to increase bacterial secretion, to facilitate antigen processing, to increase flexibility of the fusion polypeptide, to increase rigidity of the fusion polypeptide, or any other purpose. In some cases, different amino acid linker sequences are distributed between the antigenic peptides or different nucleic acids encoding the same amino acid linker sequence are distributed between the antigenic peptides (e.g., SEQ ID NOS: 84-94) in order to minimize repeats. This can also serve to reduce secondary structures, thereby allowing efficient transcription, translation, secretion, maintenance, or stabilization of the nucleic acid (e.g., plasmid) encoding the fusion polypeptide within a Lm recombinant vector strain population. Other suitable peptide linker sequences may be chosen, for example, based on one or more of the following factors: (1) their ability to adopt a flexible extended conformation; (2) their inability to adopt a secondary structure that could interact with functional epitopes on the antigenic peptides; and (3) the lack of hydrophobic or charged residues that might react with the functional epitopes. For example, peptide linker sequences may contain Gly, Asn and Ser residues. Other near neutral amino acids, such as Thr and Ala may also be used in the linker sequence. Amino acid sequences which may be usefully employed as linkers include those disclosed in Maratea et al. (1985) Gene 40:39-46; Murphy et al. (1986) Proc Natl Acad Sci USA 83:8258-8262; U.S. Pat. Nos. 4,935,233; and 4,751,180, each of which is herein incorporated by reference in its entirety for all purposes. Specific examples of linkers include those in Table 2 (each of which can be used by itself as a linker, in a linker comprising repeats of the sequence, or in a linker further comprising one or more of the other sequences in the table), although others can also be envisioned (see, e.g., Reddy Chichili et al. (2013) Protein Science 22:153-167, herein incorporated by reference in its entirety for all purposes). Unless specified, “n” represents an undetermined number of repeats in the listed linker.

TABLE 2
Linkers.

| Peptide Linker | Example | SEQ ID NO: | Hypothetical Purpose |
|---|---|---|---|
| (GAS)ₙ | GASGAS | 33 | Flexibility |
| (GSA)ₙ | GSAGSA | 34 | Flexibility |
| (G)ₙ; n = 4-8 | GGGG | 35 | Flexibility |
| (GGGGS)ₙ; n = 1-3 | GGGGS | 36 | Flexibility |
| VGKGGSGG | VGKGGSGG | 37 | Flexibility |
| (PAPAP)ₙ | PAPAP | 38 | Rigidity |
| (EAAAK)ₙ; n = 1-3 | EAAAK | 39 | Rigidity |
| (AYL)ₙ | AYLAYL | 40 | Antigen Processing |
| (LRA)ₙ | LRALRA | 41 | Antigen Processing |
| (RLRA)ₙ | RLRA | 42 | Antigen Processing |

B. PEST-Containing Peptides

The recombinant fusion proteins disclosed herein comprise a PEST-containing peptide. The PEST-containing peptide may at the amino terminal (N-terminal) end of the fusion polypeptide (i.e., N-terminal to the antigenic peptides), may be at the carboxy terminal (C-terminal) end of the fusion polypeptide (i.e., C-terminal to the antigenic peptides), or may be embedded within the antigenic peptides. In some recombinant Listeria strains and methods, a PEST containing peptide is not part of and is separate from the fusion polypeptide. Fusion of an antigenic peptides to a PEST-like sequence, such as an LLO peptide, can enhance the immunogenicity of the antigenic peptides and can increase cell-mediated and antitumor immune responses (i.e., increase cell-mediated and anti-tumor immunity). See, e.g., Singh et al. (2005) J Immunol 175(6):3663-3673, herein incorporated by reference in its entirety for all purposes.

A PEST-containing peptide is one that comprises a PEST sequence or a PEST-like sequence. PEST sequences in eukaryotic proteins have long been identified. For example, proteins containing amino acid sequences that are rich in prolines (P), glutamic acids (E), serines (S) and threonines (T) (PEST), generally, but not always, flanked by clusters containing several positively charged amino acids, have rapid intracellular half-lives (Rogers et al. (1986) Science 234:364-369, herein incorporated by reference in its entirety for all purposes). Further, it has been reported that these sequences target the protein to the ubiquitin-proteasome pathway for degradation (Rechsteiner and Rogers (1996) Trends Biochem. Sci. 21:267-271, herein incorporated by reference in its entirety for all purposes). This pathway is also used by eukaryotic cells to generate immunogenic peptides that bind to MHC class I and it has been hypothesized that PEST sequences are abundant among eukaryotic proteins that give rise to immunogenic peptides (Realini et al. (1994) FEBS Lett. 348:109-113, herein incorporated by reference in its entirety for all purposes). Prokaryotic proteins do not normally contain PEST sequences because they do not have this enzymatic pathway. However, a PEST-like sequence rich in the amino acids proline (P), glutamic acid (E), serine (S) and threonine (T) has been reported at the amino terminus of LLO and has been reported to be essential for L. monocytogenes pathogenicity (Decatur and Portnoy (2000) Science 290:992-995, herein incorporated by reference in its entirety for all purposes). The presence of this PEST-like sequence in LLO targets the protein for destruction by proteolytic machinery of the host cell so that once the LLO has served its function and facilitated the escape of L. monocytogenes from the phagosomal or phagolysosomal vacuole, it is destroyed before it can damage the cells.

Identification of PEST and PEST-like sequences is well-known and is described, for example, in Rogers et al. (1986) Science 234(4774):364-378 and in Rechsteiner and Rogers (1996) Trends Biochem. Sci. 21:267-271, each of which is herein incorporated by reference in its entirety for all purposes. A PEST or PEST-like sequence can be identified using the PEST-find program. For example, a PEST-like sequence can be a region rich in proline (P), glutamic acid (E), serine (S), and threonine (T) residues. Optionally, the PEST-like sequence can be flanked by one or more clusters containing several positively charged amino acids. For example, a PEST-like sequence can be defined as a hydrophilic stretch of at least 12 amino acids in length with a high local concentration of proline (P), aspartate (D), glutamate (E), serine (S), and/or threonine (T) residues. In some cases, a PEST-like sequence contains no positively charged amino acids, namely arginine (R), histidine (H), and lysine (K). Some PEST-like sequences can contain one or more internal phosphorylation sites, and phosphorylation at these sites precedes protein degradation.

In one example, the PEST-like sequence fits an algorithm disclosed in Rogers et al. In another example, the PEST-like sequence fits an algorithm disclosed in Rechsteiner and Rogers. PEST-like sequences can also be identified by an initial scan for positively charged amino acids R, H, and K within the specified protein sequence. All amino acids between the positively charged flanks are counted, and only those motifs containing a number of amino acids equal to or higher than the window-size parameter are considered further. Optionally, a PEST-like sequence must contain at least one P, at least one D or E, and at least one S or T.

The quality of a PEST motif can be refined by means of a scoring parameter based on the local enrichment of critical amino acids as well as the motifs hydrophobicity. Enrichment of D, E, P, S, and T is expressed in mass percent (w/w) and corrected for one equivalent of D or E, one 1 of P, and one of S or T. Calculation of hydrophobicity can also follow in principle the method of Kyte and Doolittle (1982) J. Mol. Biol. 157:105, herein incorporated by reference in its entirety for all purposes. For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine: Hydropathy index=10*Kyte-Doolittle hydropathy index+45.

A potential PEST motif's hydrophobicity can also be calculated as the sum over the products of mole percent and hydrophobicity index for each amino acid species. The desired PEST score is obtained as combination of local enrichment term and hydrophobicity term as expressed by the following equation: PEST score=0.55*DEPST−0.5*hydrophobicity index.

Thus, a PEST-containing peptide can refer to a peptide having a score of at least +5 using the above algorithm. Alternatively, it can refer to a peptide having a score of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 35, at least 38, at least 40, or at least 45.

Any other known available methods or algorithms can also be used to identify PEST-like sequences. See, e.g., the CaSPredictor (Garay-Malpartida et al. (2005) Bioinformatics 21 Suppl 1:i169-76, herein incorporated by reference in its entirety for all purposes). Another method that can be used is the following: a PEST index is calculated for each stretch of appropriate length (e.g. a 30-35 amino acid stretch) by assigning a value of one to the amino acids Ser, Thr, Pro, Glu, Asp, Asn, or Gln. The coefficient value (CV) for each of the PEST residues is one and the CV for each of the other AA (non-PEST) is zero.

Examples of PEST-like amino acid sequences are those set forth in SEQ ID NOS: 43-51. One example of a PEST-like sequence is KENSISSMAPPASPPASPKTPIEKKHADEIDK (SEQ ID NO: 43). Another example of a PEST-like sequence is KENSISSMAPPASPPASPK (SEQ ID NO: 44). However, any PEST or PEST-like amino acid sequence can be used. PEST sequence peptides are known and are described, for example, in U.S. Pat. Nos. 7,635,479; 7,665,238; and US 2014/0186387, each of which is herein incorporated by reference in its entirety for all purposes.

The PEST-like sequence can be from a Listeria species, such as from Listeria monocytogenes. For example, the Listeria monocytogenes ActA protein contains at least four such sequences (SEQ ID NOS: 45-48), any of which are suitable for use in the compositions and methods disclosed herein. Other similar PEST-like sequences include SEQ ID NOS: 52-54. Streptolysin O proteins from Streptococcus sp. also contain a PEST sequence. For example, Streptococcus pyogenes streptolysin O comprises the PEST sequence KQNTASTETTTTNEQPK (SEQ ID NO: 49) at amino acids 35-51 and Streptococcus equisimilis streptolysin O comprises the PEST-like sequence KQNTANTETTTTNEQPK (SEQ ID NO: 50) at amino acids 38-54. Another example of a PEST-like sequence is from Listeria seeligeri cytolysin, encoded by the lso gene: RSEVTISPAETPESPPATP (e.g., SEQ ID NO: 51).

Alternatively, the PEST-like sequence can be derived from other prokaryotic organisms. Other prokaryotic organisms wherein PEST-like amino acid sequences would be expected include, for example, other Listeria species.

(1) Listeriolysin O (LLO)

One example of a PEST-containing peptide that can be utilized in the compositions and methods disclosed herein is a listeriolysin O (LLO) peptide. An example of an LLO protein is the protein assigned GenBank Accession No. P13128 (SEQ ID NO: 55; nucleic acid sequence is set forth in GenBank Accession No. X15127). SEQ ID NO: 55 is a proprotein including a signal sequence. The first 25 amino acids of the proprotein is the signal sequence and is cleaved from LLO when it is secreted by the bacterium, thereby resulting in the full-length active LLO protein of 504 amino acids without the signal sequence. An LLO peptide disclosed herein can comprise the signal sequence or can comprise a peptide that does not include the signal sequence. Exemplary LLO proteins that can be used comprise, consist essentially of, or consist of the sequence set forth in SEQ ID NO: 55 or homologues, variants, isoforms, analogs, fragments, fragments of homologues, fragments of variants, fragments of analogs, and fragments of isoforms of SEQ ID NO: 55. Any sequence that encodes a fragment of an LLO protein or a homologue, variant, isoform, analog, fragment of a homologue, fragment of a variant, or fragment of an analog of an LLO protein can be used. A homologous LLO protein can have a sequence identity with a reference LLO protein, for example, of greater than 70%, 72%, 75%, 78%, 80%, 82%, 83%, 85%, 87%, 88%, 90%, 92%, 93%, 95%, 96%, 97%, 98%, or 99%.

Another example of an LLO protein is set forth in SEQ ID NO: 56. LLO proteins that can be used can comprise, consist essentially of, or consist of the sequence set forth in SEQ ID NO: 56 or homologues, variants, isoforms, analogs, fragments, fragments of homologues, fragments of variants, fragments of analogs, and fragments of isoforms of SEQ ID NO: 56.

Another example of an LLO protein is an LLO protein from the Listeria monocytogenes 10403S strain, as set forth in GenBank Accession No.: ZP_01942330 or EBA21833, or as encoded by the nucleic acid sequence as set forth in GenBank Accession No.: NZ_AARZ01000015 or AARZ01000015.1. Another example of an LLO protein is an LLO protein from the Listeria monocytogenes 4b F2365 strain (see, e.g., GenBank Accession No.: YP_012823), EGD-e strain (see, e.g., GenBank Accession No.: NP_463733), or any other strain of Listeria monocytogenes. Yet another example of an LLO protein is an LLO protein from Flavobacteriales bacterium HTCC2170 (see, e.g., GenBank Accession No.: ZP_01106747 or EAR01433, or encoded by GenBank Accession No.: NZ_AAOC01000003). LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

Proteins that are homologous to LLO, or homologues, variants, isoforms, analogs, fragments, fragments of homologues, fragments of variants, fragments of analogs, and fragments of isoforms thereof, can also be used. One such example is alveolysin, which can be found, for example, in Paenibacillus alvei (see, e.g., GenBank Accession No.: P23564 or AAA22224, or encoded by GenBank Accession No.: M62709). Other such homologous proteins are known.

The LLO peptide can be a full-length LLO protein or a truncated LLO protein or LLO fragment. Likewise, the LLO peptide can be one that retains one or more functionalities of a native LLO protein or lacks one or more functionalities of a native LLO protein. For example, the retained LLO functionality can be allowing a bacteria (e.g., Listeria) to escape from a phagosome or phagolysosome, or enhancing the immunogenicity of a peptide to which it is fused. The retained functionality can also be hemolytic function or antigenic function. Alternatively, the LLO peptide can be a non-hemolytic LLO. Other functions of LLO are known, as are methods and assays for evaluating LLO functionality.

An LLO fragment can be a PEST-like sequence or can comprise a PEST-like sequence. LLO fragments can comprise one or more of an internal deletion, a truncation from the C-terminal end, and a truncation from the N-terminal end. In some cases, an LLO fragment can comprise more than one internal deletion. Other LLO peptides can be full-length LLO proteins with one or more mutations.

Some LLO proteins or fragments have reduced hemolytic activity relative to wild type LLO or are non-hemolytic fragments. For example, an LLO protein can be rendered non-hemolytic by deletion or mutation of the activation domain at the carboxy terminus, by deletion or mutation of cysteine 484, or by deletion or mutation at another location.

Other LLO proteins are rendered non-hemolytic by a deletion or mutation of the cholesterol binding domain (CBD) as detailed in U.S. Pat. No. 8,771,702, herein incorporated by reference in its entirety for all purposes. The mutations can comprise, for example, a substitution or a deletion. The entire CBD can be mutated, portions of the CBD can be mutated, or specific residues within the CBD can be mutated. For example, the LLO protein can comprise a mutation of one or more of residues C484, W491, and W492 (e.g., C484, W491, W492, C484 and W491, C484 and W492, W491 and W492, or all three residues) of SEQ ID NO: 55 or corresponding residues when optimally aligned with SEQ ID NO: 55 (e.g., a corresponding cysteine or tryptophan residue). As an example, a mutant LLO protein can be created wherein residues C484, W491, and W492 of LLO are substituted with alanine residues, which will substantially reduce hemolytic activity relative to wild type LLO. The mutant LLO protein with C484A, W491A, and W492A mutations is termed “mutLLO.”

As another example, a mutant LLO protein can be created with an internal deletion comprising the cholesterol-binding domain. The sequence of the cholesterol-binding domain of SEQ ID NO: 55 set forth in SEQ ID NO: 74. For example, the internal deletion can be a 1-11 amino acid deletion, an 11-50 amino acid deletion, or longer. Likewise, the mutated region can be 1-11 amino acids, 11-50 amino acids, or longer (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). For example, a mutated region consisting of residues 470-500, 470-510, or 480-500 of SEQ ID NO: 55 will result in a deleted sequence comprising the CBD (residues 483-493 of SEQ ID NO: 55). However, the mutated region can also be a fragment of the CBD or can overlap with a portion of the CBD. For example, the mutated region can consist of residues 470-490, 480-488, 485-490, 486-488, 490-500, or 486-510 of SEQ ID NO: 55. For example, a fragment of the CBD (residues 484-492) can be replaced with a heterologous sequence, which will substantially reduce hemolytic activity relative to wild type LLO. For example, the CBD (ECTGLAWEWWR; SEQ ID NO: 74) can be replaced with a CTL epitope from the antigen NY-ESO-1 (ESLLMWITQCR; SEQ ID NO: 75), which contains the HLA-A2 restricted epitope 157-165 from NY-ESO-1. The resulting LLO is termed “ctLLO.”

In some mutated LLO proteins, the mutated region can be replaced by a heterologous sequence. For example, the mutated region can be replaced by an equal number of heterologous amino acids, a smaller number of heterologous amino acids, or a larger number of amino acids (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). Other mutated LLO proteins have one or more point mutations (e.g., a point mutation of 1 residue, 2 residues, 3 residues, or more). The mutated residues can be contiguous or not contiguous.

In one example embodiment, an LLO peptide may have a deletion in the signal sequence and a mutation or substitution in the CBD.

Some LLO peptides are N-terminal LLO fragments (i.e., LLO proteins with a C-terminal deletion). Some LLO peptides are at least 494, 489, 492, 493, 500, 505, 510, 515, 520, or 525 amino acids in length or 492-528 amino acids in length. For example, the LLO fragment can consist of about the first 440 or 441 amino acids of an LLO protein (e.g., the first 441 amino acids of SEQ ID NO: 55 or 56, or a corresponding fragment of another LLO protein when optimally aligned with SEQ ID NO: 55 or 56). Other N-terminal LLO fragments can consist of the first 420 amino acids of an LLO protein (e.g., the first 420 amino acids of SEQ ID NO: 55 or 56, or a corresponding fragment of another LLO protein when optimally aligned with SEQ ID NO: 55 or 56). Other N-terminal fragments can consist of about amino acids 20-442 of an LLO protein (e.g., amino acids 20-442 of SEQ ID NO: 55 or 56, or a corresponding fragment of another LLO protein when optimally aligned with SEQ ID NO: 55 or 56). Other N-terminal LLO fragments comprise any ΔLLO without the activation domain comprising cysteine 484, and in particular without cysteine 484. For example, the N-terminal LLO fragment can correspond to the first 425, 400, 375, 350, 325, 300, 275, 250, 225, 200, 175, 150, 125, 100, 75, 50, or 25 amino acids of an LLO protein (e.g., the first 425, 400, 375, 350, 325, 300, 275, 250, 225, 200, 175, 150, 125, 100, 75, 50, or 25 amino acids of SEQ ID NO: 55 or 56, or a corresponding fragment of another LLO protein when optimally aligned with SEQ ID NO: 55 or 56). Preferably, the fragment comprises one or more PEST-like sequences. LLO fragments and truncated LLO proteins can contain residues of a homologous LLO protein that correspond to any one of the above specific amino acid ranges. The residue numbers need not correspond exactly with the residue numbers enumerated above (e.g., if the homologous LLO protein has an insertion or deletion relative to a specific LLO protein disclosed herein). Examples of N-terminal LLO fragments include SEQ ID NOS: 57, 58, and 59. LLO proteins that can be used comprise, consist essentially of, or consist of the sequence set forth in SEQ ID NO: 57, 58, or 59 or homologues, variants, isoforms, analogs, fragments, fragments of homologues, fragments of variants, fragments of analogs, and fragments of isoforms of SEQ ID NO: 57, 58, or 59. In some compositions and methods, the N-terminal LLO fragment set forth in SEQ ID NO: 59 is used. An example of a nucleic acid encoding the N-terminal LLO fragment set forth in SEQ ID NO: 59 is SEQ ID NO: 60.

(2) ActA

Another example of a PEST-containing peptide that can be utilized in the compositions and methods disclosed herein is an ActA peptide. ActA is a surface-associated protein and acts as a scaffold in infected host cells to facilitate the polymerization, assembly, and activation of host actin polymers in order to propel a Listeria monocytogenes through the cytoplasm. Shortly after entry into the mammalian cell cytosol, L. monocytogenes induces the polymerization of host actin filaments and uses the force generated by actin polymerization to move, first intracellularly and then from cell to cell. ActA is responsible for mediating actin nucleation and actin-based motility. The ActA protein provides multiple binding sites for host cytoskeletal components, thereby acting as a scaffold to assemble the cellular actin polymerization machinery. The N-terminus of ActA binds to monomeric actin and acts as a constitutively active nucleation promoting factor by stimulating the intrinsic actin nucleation activity. The actA and hly genes are both members of the 10-kb gene cluster regulated by the transcriptional activator PrfA, and actA is upregulated approximately 226-fold in the mammalian cytosol. Any sequence that encodes an ActA protein or a homologue, variant, isoform, analog, fragment of a homologue, fragment of a variant, or fragment of an analog of an ActA protein can be used. A homologous ActA protein can have a sequence identity with a reference ActA protein, for example, of greater than 70%, 72%, 75%, 78%, 80%, 82%, 83%, 85%, 87%, 88%, 90%, 92%, 93%, 95%, 96%, 97%, 98%, or 99%.

One example of an ActA protein comprises, consists essentially of, or consists of the sequence set forth in SEQ ID NO: 61. Another example of an ActA protein comprises, consists essentially of, or consists of the sequence set forth in SEQ ID NO: 62. The first 29 amino acid of the proprotein corresponding to either of these sequences are the signal sequence and are cleaved from ActA protein when it is secreted by the bacterium. An ActA peptide can comprise the signal sequence (e.g., amino acids 1-29 of SEQ ID NO: 61 or 62), or can comprise a peptide that does not include the signal sequence. Other examples of ActA proteins comprise, consist essentially of, or consist of homologues, variants, isoforms, analogs, fragments, fragments of homologues, fragments of isoforms, or fragments of analogs of SEQ ID NO: 61 or 62.

Another example of an ActA protein is an ActA protein from the Listeria monocytogenes 10403S strain (GenBank Accession No.: DQ054585) the NICPBP 54002 strain (GenBank Accession No.: EU394959), the S3 strain (GenBank Accession No.: EU394960), NCTC 5348 strain (GenBank Accession No.: EU394961), NICPBP 54006 strain (GenBank Accession No.: EU394962), M7 strain (GenBank Accession No.: EU394963), S19 strain (GenBank Accession No.: EU394964), or any other strain of Listeria monocytogenes. LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

ActA peptides can be full-length ActA proteins or truncated ActA proteins or ActA fragments (e.g., N-terminal ActA fragments in which a C-terminal portion is removed). Preferably, truncated ActA proteins comprise at least one PEST sequence (e.g., more than one PEST sequence). In addition, truncated ActA proteins can optionally comprise an ActA signal peptide. Examples of PEST-like sequences contained in truncated ActA proteins include SEQ ID NOS: 45-48. Some such truncated ActA proteins comprise at least two of the PEST-like sequences set forth in SEQ ID NOS: 45-48 or homologs thereof, at least three of the PEST-like sequences set forth in SEQ ID NOS: 45-48 or homologs thereof, or all four of the PEST-like sequences set forth in SEQ ID NOS: 45-48 or homologs thereof. Examples of truncated ActA proteins include those comprising, consisting essentially of, or consisting of about residues 30-122, about residues 30-229, about residues 30-332, about residues 30-200, or about residues 30-399 of a full length ActA protein sequence (e.g., SEQ ID NO: 62). Other examples of truncated ActA proteins include those comprising, consisting essentially of, or consisting of about the first 50, 100, 150, 200, 233, 250, 300, 390, 400, or 418 residues of a full length ActA protein sequence (e.g., SEQ ID NO: 62). Other examples of truncated ActA proteins include those comprising, consisting essentially of, or consisting of about residues 200-300 or residues 300-400 of a full length ActA protein sequence (e.g., SEQ ID NO: 62). For example, the truncated ActA consists of the first 390 amino acids of the wild type ActA protein as described in U.S. Pat. No. 7,655,238, herein incorporated by reference in its entirety for all purposes. As another example, the truncated ActA can be an ActA-N100 or a modified version thereof (referred to as ActA-N100*) in which a PEST motif has been deleted and containing the nonconservative QDNKR (SEQ ID NO: 73) substitution as described in US 2014/0186387, herein incorporated by references in its entirety for all purposes. Alternatively, truncated ActA proteins can contain residues of a homologous ActA protein that corresponds to one of the above amino acid ranges or the amino acid ranges of any of the ActA peptides disclosed herein. The residue numbers need not correspond exactly with the residue numbers enumerated herein (e.g., if the homologous ActA protein has an insertion or deletion, relative to an ActA protein utilized herein, then the residue numbers can be adjusted accordingly).

Examples of truncated ActA proteins include, for example, proteins comprising, consisting essentially of, or consisting of the sequence set forth in SEQ ID NO: 63, 64, 65, or 66 or homologues, variants, isoforms, analogs, fragments of variants, fragments of isoforms, or fragments of analogs of SEQ ID NO: 63, 64, 65, or 66. SEQ ID NO: 63 referred to as ActA/PEST1 and consists of amino acids 30-122 of the full length ActA sequence set forth in SEQ ID NO: 62. SEQ ID NO: 64 is referred to as ActA/PEST2 or LA229 and consists of amino acids 30-229 of the full length ActA sequence set forth in the full-length ActA sequence set forth in SEQ ID NO: 62. SEQ ID NO: 65 is referred to as ActA/PEST3 and consists of amino acids 30-332 of the full-length ActA sequence set forth in SEQ ID NO: 62. SEQ ID NO: 66 is referred to as ActA/PEST4 and consists of amino acids 30-399 of the full-length ActA sequence set forth in SEQ ID NO: 62. As a specific example, the truncated ActA protein consisting of the sequence set forth in SEQ ID NO: 64 can be used.

Examples of truncated ActA proteins include, for example, proteins comprising, consisting essentially of, or consisting of the sequence set forth in SEQ ID NO: 67, 69, 70, or 72 or homologues, variants, isoforms, analogs, fragments of variants, fragments of isoforms, or fragments of analogs of SEQ ID NO: 67, 69, 70, or 72. As a specific example, the truncated ActA protein consisting of the sequence set forth in SEQ ID NO: 67 (encoded by the nucleic acid set forth in SEQ ID NO: 68) can be used. As another specific example, the truncated ActA protein consisting of the sequence set forth in SEQ ID NO: 70 (encoded by the nucleic acid set forth in SEQ ID NO: 71) can be used. SEQ ID NO: 71 is the first 1170 nucleotides encoding ActA in the Listeria monocytogenes 10403S strain. In some cases, the ActA fragment can be fused to a heterologous signal peptide. For example, SEQ ID NO: 72 sets forth an ActA fragment fused to an Hly signal peptide.

C. Generating Immunotherapy Constructs Encoding Recombinant Fusion Polypeptides

Also provided herein are methods for generating immunotherapy constructs encoding or compositions comprising the recombinant fusion polypeptides disclosed herein. For example, such methods can comprise selecting and designing antigenic peptides to include in the immunotherapy construct (and, for example, testing the hydropathy of the each antigenic peptide, and modifying or deselecting an antigenic peptide if it scores above a selected hydropathy index threshold value), designing one or more fusion polypeptides comprising each of the selected antigenic peptides, and generating a nucleic acid construct encoding the fusion polypeptide.

The antigenic peptides can be screened for hydrophobicity or hydrophilicity. Antigenic peptides can be selected, for example, if they are hydrophilic or if they score up to or below a certain hydropathy threshold, which can be predictive of secretability in a particular bacteria of interest (e.g., Listeria monocytogenes). For example, antigenic peptides can be scored by Kyte and Doolittle hydropathy index with a 21 amino acid window, all scoring above cutoff (around 1.6) are excluded as they are unlikely to be secretable by Listeria monocytogenes. See, e.g., Kyte-Doolittle (1982) J Mol Biol 157(1):105-132; herein incorporated by reference in its entirety for all purposes. Alternatively, an antigenic peptide scoring about a selected cutoff can be altered (e.g., changing the length of the antigenic peptide). Other sliding window sizes that can be used include, for example, 9, 11, 13, 15, 17, 19, 21, 23, 25, 27 or more amino acids. For example, the sliding window size can be 9-11 amino acids, 11-13 amino acids, 13-15 amino acids, 15-17 amino acids, 17-19 amino acids, 19-21 amino acids, 21-23 amino acids, 23-25 amino acids, or 25-27 amino acids. Other cutoffs that can be used include, for example, the following ranges 1.2-1.4, 1.4-1.6, 1.6-1.8, 1.8-2.0, 2.0-2.2 2.2-2.5, 2.5-3.0, 3.0-3.5, 3.5-4.0, or 4.0-4.5, or the cutoff can be 1.4, 1.5, 1.7, 1.8, 1.9, 2.0, 2.1, 2.2, 2.3, 2.3, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. The cutoff can vary, for example, depending on the genus or species of the bacteria being used to deliver the fusion polypeptide.

Other suitable hydropathy plots or other appropriate scales include, for example, those reported in Rose et al. (1993) Annu Rev Biomol Struct 22:381-415; Biswas et al. (2003) Journal of Chromatography A 1000:637-655; Eisenberg (1984) Ann Rev Biochem 53:595-623; Abraham and Leo (1987) Proteins: Structure, Function and Genetics 2:130-152; Sweet and Eisenberg (1983) Mol Biol 171:479-488; Bull and Breese (1974) Arch Biochem Biophys 161:665-670; Guy (1985) Biophys J 47:61-70; Miyazawa et al. (1985) Macromolecules 18:534-552; Roseman (1988) J Mol Biol 200:513-522; Wolfenden et al. (1981) Biochemistry 20:849-855; Wilson (1981) Biochem J 199:31-41; Cowan and Whittaker (1990) Peptide Research 3:75-80; Aboderin (1971) Int J Biochem 2:537-544; Eisenberg et al. (1984) J Mol Biol 179:125-142; Hopp and Woods (1981) Proc Natl Acad Sci USA 78:3824-3828; Manavalan and Ponnuswamy (1978) Nature 275:673-674; Black and Mould (1991) Anal Biochem 193:72-82; Fauchere and Pliska (1983) Eur J Med Chem 18:369-375; Janin (1979) Nature 277:491-492; Rao and Argos (1986) Biochim Biophys Acta 869:197-214; Tanford (1962) Am Chem Soc 84:4240-4274; Welling et al. (1985) FEBS Lett 188:215-218; Parker et al. (1986) Biochemistry 25:5425-5431; and Cowan and Whittaker (1990) Peptide Research 3:75-80, each of which is herein incorporated by reference in its entirety for all purposes.

Optionally, the antigenic peptides can be scored for their ability to bind to the subject human leukocyte antigen (HLA) type (for example by using the Immune Epitope Database (IED) available at www.iedb.org, which includes netMHCpan, ANN, SMMPMBEC. SMM, CombLib_Sidney2008, PickPocket, and netMHCcons) and ranked by best MHC binding score from each antigenic peptide. Other sources include TEpredict (tepredict.sourceforge.net/help.html) or other available MHC binding measurement scales. Cutoffs may be different for different expression vectors such as Salmonella.

Optionally, the antigenic peptides can be screened for immunosuppressive epitopes (e.g., T-reg epitopes, IL-10-inducing T helper epitopes, and so forth) to deselect antigenic peptides or to avoid immunosuppressive influences.

Optionally, a predicative algorithm for immunogenicity of the epitopes can be used to screen the antigenic peptides. However, these algorithms are at best 20% accurate in predicting which peptide will generate a T cell response. Alternatively, no screening/predictive algorithms are used. Alternatively, the antigenic peptides can be screened for immunogenicity. For example, this can comprise contacting one or more T cells with an antigenic peptide, and analyzing for an immunogenic T cell response, wherein an immunogenic T cell response identifies the peptide as an immunogenic peptide. This can also comprise using an immunogenic assay to measure secretion of at least one of CD25, CD44, or CD69 or to measure secretion of a cytokine selected from the group comprising IFN-γ, TNF-α, IL-1, and IL-2 upon contacting the one or more T cells with the peptide, wherein increased secretion identifies the peptide as comprising one or more T cell epitopes.

The selected antigenic peptides can be arranged into one or more candidate orders for a potential fusion polypeptide. If there are more usable antigenic peptides than can fit into a single plasmid, different antigenic peptides can be assigned priority ranks as needed/desired and/or split up into different fusion polypeptides (e.g., for inclusion in different recombinant Listeria strains). Priority rank can be determined by factors such as relative size, priority of transcription, and/or overall hydrophobicity of the translated polypeptide. The antigenic peptides can be arranged so that they are joined directly together without linkers, or any combination of linkers between any number of pairs of antigenic peptides, as disclosed in more detail elsewhere herein. The number of linear antigenic peptides to be included can be determined based on consideration of the number of constructs needed versus the mutational burden, the efficiency of translation and secretion of multiple epitopes from a single plasmid, and the MOI needed for each bacteria or Lm comprising a plasmid.

The combination of antigenic peptides or the entire fusion polypeptide (i.e., comprising the antigenic peptides and the PEST-containing peptide and any tags) also be scored for hydrophobicity. For example, the entirety of the fused antigenic peptides or the entire fusion polypeptide can be scored for hydropathy by a Kyte and Doolittle hydropathy index with a sliding 21 amino acid window. If any region scores above a cutoff (e.g., around 1.6), the antigenic peptides can be reordered or shuffled within the fusion polypeptide until an acceptable order of antigenic peptides is found (i.e., one in which no region scores above the cutoff). Alternatively, any problematic antigenic peptides can be removed or redesigned to be of a different size. Alternatively or additionally, one or more linkers between antigenic peptides as disclosed elsewhere herein can be added or modified to change the hydrophobicity. As with hydropathy testing for the individual antigenic peptides, other window sizes can be used, or other cutoffs can be used (e.g., depending on the genus or species of the bacteria being used to deliver the fusion polypeptide). In addition, other suitable hydropathy plots or other appropriate scales could be used.

Optionally, the combination of antigenic peptides or the entire fusion polypeptide can be further screened for immunosuppressive epitopes (e.g., T-reg epitopes, IL-10-inducing T helper epitopes, and so forth) to deselect antigenic peptides or to avoid immunosuppressive influences.

A nucleic acid encoding a candidate combination of antigenic peptides or fusion polypeptide can then be designed and optimized. For example, the sequence can be optimized for increased levels of translation, duration of expression, levels of secretion, levels of transcription, and any combination thereof. For example, the increase can be 2-fold to 1000-fold, 2-fold to 500-fold, 2-fold to 100-fold, 2-fold to 50-fold, 2-fold to 20-fold, 2-fold to 10-fold, or 3-fold to 5-fold relative to a control, non-optimized sequence.

For example, the fusion polypeptide or nucleic acid encoding the fusion polypeptide can be optimized for decreased levels of secondary structures possibly formed in the oligonucleotide sequence, or alternatively optimized to prevent attachment of any enzyme that may modify the sequence. Expression in bacterial cells can be hampered, for example, by transcriptional silencing, low mRNA half-life, secondary structure formation, attachment sites of oligonucleotide binding molecules such as repressors and inhibitors, and availability of rare tRNAs pools. The source of many problems in bacterial expressions is found within the original sequence. The optimization of RNAs may include modification of cis acting elements, adaptation of its GC-content, modifying codon bias with respect to non-limiting tRNAs pools of the bacterial cell, and avoiding internal homologous regions. Thus, optimizing a sequence can entail, for example, adjusting regions of very high (>80%) or very low (<30%) GC content. Optimizing a sequence can also entail, for example, avoiding one or more of the following cis-acting sequence motifs: internal TATA-boxes, chi-sites, and ribosomal entry sites; AT-rich or GC-rich sequence stretches; repeat sequences and RNA secondary structures; (cryptic) splice donor and acceptor sites; branch points; or a combination thereof. Optimizing expression can also entail adding sequence elements to flanking regions of a gene and/or elsewhere in the plasmid.

Optimizing a sequence can also entail, for example, adapting the codon usage to the codon bias of host genes (e.g., Listeria monocytogenes genes). For example, the codons below can be used for Listeria monocytogenes.

TABLE 3
Codons.
A = GCA
C = TGT
D = GAT
E = GAA
F = TTC
G = GGT
H = CAT
I = ATT
K = AAA
L = TTA
M = ATG
N = AAC
P = CCA
Q = CAA
R = CGT
S = TCT
T = ACA
V = GTT
W = TGG
Y = TAT
STOP = TAA

A nucleic acid encoding a fusion polypeptide can be generated and introduced into a delivery vehicle such as a bacteria strain or Listeria strain. Other delivery vehicles may be suitable for DNA immunotherapy or peptide immunotherapy, such as a vaccinia virus or virus-like particle. Once a plasmid encoding a fusion polypeptide is generated and introduced into a bacteria strain or Listeria strain, the bacteria or Listeria strain can be cultured and characterized to confirm expression and secretion of the fusion polypeptide comprising the antigenic peptides.

### V. Kits

Also provided are kits comprising a one or more reagents utilized in performing any of the methods disclosed herein or kits comprising any of the compositions, tools, or instruments disclosed herein.

For example, such kits can comprise THP-1 cells and optionally, one or more reagents or instructional materials for differentiating the THP-1 cells. Such kits can also comprise a recombinant bacteria or Listeria strain disclosed herein. In addition, such kits can additionally comprise an instructional material which describes use of the THP-1 cells and/or recombinant bacteria or Listeria strain to perform the methods disclosed herein. Although model kits are described below, the contents of other useful kits will be apparent in light of the present disclosure.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail byway of illustration and example for purposes of clarity and understanding, it will be apparent that certain changes and modifications may be practiced within the scope of the appended claims.

### LISTING OF EMBODIMENTS

The subject matter disclosed herein includes, but is not limited to, the following embodiments.

1. A method of assessing attenuation or infectivity of a test Listeria strain, comprising:

(a) infecting differentiated THP-1 cells with the test Listeria strain, wherein the TIP-1 cells have been differentiated into macrophages prior to infecting with the test Listeria strain;

(b) lysing the TIP-1 cells and plating the lysate on agar; and

(c) counting the Listeria that have multiplied inside the TIP-1 cells by growth on the agar.

2. The method of embodiment 1, further comprising differentiating the THP-1 cells into macrophages using phorbol 12-myristate 13-acetate (PMA) prior to step (a).

3. The method of embodiment 1 or 2, wherein infecting differentiated THP-1 cells with the test Listeria strain comprises: inoculating the differentiated TIP-1 cells with the test Listeria strain and incubating the test Listeria strain with the differentiated THP-1 cells for 1-5 hours to form infected TIP1 cells.

4. The method of any preceding embodiment, wherein step (a) comprises infecting the differentiated TIP-1 cells at a multiplicity of infection (MOI) of 1:1.

5. The method of any preceding embodiment, further comprising killing Listeria not taken up by the TIP-1 cells in between steps (a) and (b).

6. The method of embodiment 5, wherein the killing is performed using an antibiotic, optionally wherein the antibiotic is gentamicin.

7. The method of any one of embodiments 1-4, wherein extracellular Listeria are removed from infected TIP-1 cells prior to step (b).

8. The method of embodiment 7, wherein removing extracellular Listeria comprises adding an antibiotic effective against the Listeria, optionally wherein the antibiotic is gentamicin.

9. The method of embodiment 7 or 8, wherein infected THP-1 cells are incubated in growth media for 0-10 hours after removing extracellular Listeria and before step (b).

10. The method of any preceding embodiment, wherein step (b) is performed at 0 hours post-infection.

11. The method of any preceding embodiment, wherein step (b) is performed at 0 hours post-infection, 1 hour post-infection, 3 hours post-infection and/or 5 hours post-infection.

12. The method of any preceding embodiment, wherein the agar contains a media capable of supporting growth of the Listeria.

13. The method of any preceding embodiment, further comprising comparing uptake and intracellular growth of the test Listeria strain with a wild type Listeria strain and/or a reference sample.

14. The method of any preceding embodiment, wherein the test Listeria strain is a Listeria monocytogenes strain.

15. The method of any preceding embodiment, wherein the test Listeria strain is a recombinant Listeria strain comprising a nucleic acid comprising a first open reading frame encoding a fusion polypeptide, wherein the fusion polypeptide comprises a PEST-containing peptide fused to a disease-associated antigenic peptide.

16. The method of embodiment 15, wherein the PEST-containing peptide is listeriolysin O (LLO) or a fragment thereof, and the disease-associated antigenic peptide is a Human Papilloma virus (HPV) protein E7 or a fragment thereof.

17. The method of embodiment 15 or 16, wherein the recombinant Listeria strain is an attenuated Listeria monocytogenes strain comprising a deletion of or inactivating mutation in prfA, wherein the nucleic acid is in an episomal plasmid and comprises a second open reading frame encoding a D133V PrfA mutant protein.

18. The method of embodiment 15, wherein the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of listeriolysin O (LLO).

19. A method of assessing attenuation or infectivity of a test bacteria strain, comprising:

(a) differentiating THP-1 cells;

(b) infecting the differentiated THP-1 cells with the test bacteria strain, wherein the infecting comprises:
(i) inoculating the differentiated THP-1 cells with the test bacteria strain;
(ii) incubating the test bacteria strain with the differentiated THP-1 cells for 1-5 hours to form infected THP1 cells;
(iii) removing extracellular bacteria from the infected THP-1 cells; and
(iv) incubating the infected TIP-1 cells in growth media for 0-10 hours;

(c) lysing the infected THP-1 cells to form a lysate;

(d) plating the lysate or a dilution of the lysate on a plate containing media capable of supporting growth of the bacteria; and

(e) counting colony forming units of the bacteria on the plate.

20. The method of embodiment 19, wherein the step of infecting the differentiated THP-1 cells is at a multiplicity of infection (MOI) of 1:1.

21. The method of embodiment 19 or 20, wherein the step of removing extracellular bacteria comprises adding an antibiotic effective against the bacteria, optionally wherein the antibiotic is gentamicin.

22. The method any one of embodiments 19-21, wherein the infected TIP-1 cells are incubated in growth media for 0, 1, 3, or 5 hours.

23. The method any one of embodiments 19-22, wherein the test bacteria strain is an L. monocytogenes strain.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

TABLE 4
Description of Sequences.

| SEQ ID NO | Type | Description |
|---|---|---|
| 1 | DNA | SENFEKL Tag v1 |
| 2 | DNA | SENFEKL Tag v2 |
| 3 | DNA | SENFEKL Tag v3 |
| 4 | DNA | SENFEKL Tag v4 |
| 5 | DNA | SENFEKL Tag v5 |
| 6 | DNA | SENFEKL Tag v6 |
| 7 | DNA | SENFEKL Tag v7 |
| 8 | DNA | SENFEKL Tag v8 |
| 9 | DNA | SENFEKL Tag v9 |
| 10 | DNA | SENFEKL Tag v10 |
| 11 | DNA | SENFEKL Tag v11 |
| 12 | DNA | SENFEKL Tag v12 |
| 13 | DNA | SENFEKL Tag v13 |
| 14 | DNA | SENFEKL Tag v14 |
| 15 | DNA | SENFEKL Tag v15 |
| 16 | Protein | SENFEKL Tag |
| 17 | DNA | RFLAG Tag v1 |
| 18 | DNA | RFLAG Tag v2 |
| 19 | DNA | RFLAG Tag v3 |
| 20 | DNA | RFLAG Tag v4 |
| 21 | DNA | RFLAG Tag v5 |
| 22 | DNA | RFLAG Tag v6 |
| 23 | DNA | RFLAG Tag v7 |
| 24 | DNA | RFLAG Tag v8 |
| 25 | DNA | RFLAG Tag v9 |
| 26 | DNA | RFLAG Tag v10 |
| 27 | DNA | RFLAG Tag v11 |
| 28 | DNA | RFLAG Tag v12 |
| 29 | DNA | RFLAG Tag v13 |
| 30 | DNA | RFLAG Tag v14 |
| 31 | DNA | RFLAG Tag v15 |
| 32 | Protein | RFLAG Tag |
| 33 | Protein | Peptide Linker v1 |
| 34 | Protein | Peptide Linker v2 |
| 35 | Protein | Peptide Linker v3 |
| 36 | Protein | Peptide Linker v4 |
| 37 | Protein | Peptide Linker v5 |
| 38 | Protein | Peptide Linker v6 |
| 39 | Protein | Peptide Linker v7 |
| 40 | Protein | Peptide Linker v8 |
| 41 | Protein | Peptide Linker v9 |
| 42 | Protein | Peptide Linker v10 |
| 43 | Protein | PEST-Like Sequence v1 |
| 44 | Protein | PEST-Like Sequence v2 |
| 45 | Protein | PEST-Like Sequence v3 |
| 46 | Protein | PEST-Like Sequence v4 |
| 47 | Protein | PEST-Like Sequence v5 |
| 48 | Protein | PEST-Like Sequence v6 |
| 49 | Protein | PEST-Like Sequence v7 |
| 50 | Protein | PEST-Like Sequence v8 |
| Si | Protein | PEST-Like Sequence v9 |
| 52 | Protein | PEST-Like Sequence v10 |
| 53 | Protein | PEST-Like Sequence v11 |
| 54 | Protein | PEST-Like Sequence v12 |
| 55 | Protein | LLO Protein v1 |
| 56 | Protein | LLO Protein v2 |
| 57 | Protein | N-Terminal Truncated LLO v1 |
| 58 | Protein | N-Terminal Truncated LLO v2 |
| 59 | Protein | N-Terminal Truncated LLO v3 |
| 60 | DNA | Nucleic Acid Encoding N-Terminal |
| | | Truncated LLO v3 |
| 61 | Protein | ActA Protein v1 |
| 62 | Protein | ActA Protein v2 |
| 63 | Protein | ActA Fragment v1 |
| 64 | Protein | ActA Fragment v2 |
| 65 | Protein | ActA Fragment v3 |
| 66 | Protein | ActA Fragment v4 |
| 67 | Protein | ActA Fragment v5 |
| 68 | DNA | Nucleic Acid Encoding ActA |
| | | Fragment v5 |
| 69 | Protein | ActA Fragment v6 |
| 70 | Protein | ActA Fragment v7 |
| 71 | DNA | Nucleic Acid Encoding ActA |
| | | Fragment v7 |
| 72 | Protein | ActA Fragment Fused to |
| | | Hly Signal Peptide |
| 73 | Protein | ActA Substitution |
| 74 | Protein | Cholesterol-Binding Domain of LLO |
| 75 | Protein | HLA-A2 restricted Epitope from |
| | | NY-ESO-1 |
| 76 | Protein | Lm Alanine Racemase |
| 77 | Protein | Lm D-Amino Acid Aminotransferase |
| 78 | DNA | Nucleic Acid Encoding Lm Alanine |
| | | Racemase |
| 79 | DNA | Nucleic Acid Encoding Lm D-Amino |
| | | Acid Aminotransferase |
| 80 | Protein | Wild Type PrfA |
| 81 | DNA | Nucleic Acid Encoding Wild Type PrfA |
| 82 | Protein | D133V PrfA |
| 83 | DNA | Nucleic Acid Encoding D133V PrfA |
| 84 | DNA | 4X Glycine Linker G1 |
| 85 | DNA | 4X Glycine Linker G2 |
| 86 | DNA | 4X Glycine Linker G3 |
| 87 | DNA | 4X Glycine Linker G4 |
| 88 | DNA | 4X Glycine Linker G5 |
| 89 | DNA | 4X Glycine Linker G6 |
| 90 | DNA | 4X Glycine Linker G7 |
| 91 | DNA | 4X Glycine Linker G8 |
| 92 | DNA | 4X Glycine Linker G9 |
| 93 | DNA | 4X Glycine Linker G10 |
| 94 | DNA | 4X Glycine Linker G11 |
| 95 | Protein | Detoxified Listeriolysin O (dtLLO) |
| 96 | Protein | Modified Cholesterol-Binding Domain |
| | | of dtLLO |
| 97 | Protein | LLO Signal Sequence |
| 98 | Protein | ActA Signal Sequence |
| 99 | Protein | Variant FLAG Tag |

### EXAMPLES

### Example 1. THP-1-Based Assays for Quantifying Intracellular Growth of Listeria monocytogenes

This example provides methods for quantifying the infection rate and/or intracellular growth of wild type Listeria monocytogenes and attenuated, recombinant Listeria monocytogenes. Cell-based assays, using differentiated TIP-1 cells, are used to analyze intracellular growth of Listeria based immunotherapies, quantitating bacteria post-infection by growth on brain heart infusion agar. In some embodiments, the described procedures are applicable to samples of ADXS11-001 or other Listeria strains.

Listeria monocytogenes is the Gram positive, non-spore forming bacterial organism that is responsible for listeriosis in humans. L. monocytogenes survives in vivo by escape from phagosomes within human macrophages. Once escaped, L. monocytogenes is able to replicate intracellularly within the cytosol of its host. The immunotherapy strain Lm-LLOE7 (e.g., ADXS11-001 L. monocytogenes, a live attenuated strain) contains a plasmid for the expression of a recombinant protein of interest (i.e., human papillomavirus protein E7 fused to truncated Listeriolysin O (tLLO)). The bacterial strain used in the Lm-LLOE7 immunotherapy is mutant strain, XFL-7, lacking the essential virulence gene prfA. The prfA gene is a transcription factor that acts on a number of genes including all of the virulence genes such as actA and hly (the gene that encodes LLO) but it is not required for in vitro culture of Listeria. XFL-7 is a virulent and can be taken up by macrophages but cannot escape the phagosome to multiply in the cytosol of macrophage. In order to evaluate attenuation of Lm-LLOE7, infection and replication are assessed in a macrophage cell infection assay, in parallel with wild type L. monocytogenes.

The recombinant protein is expressed from plasmid pGG55 containing a fusion of inactive LLO and HPV E7 coding sequences under the control of the hly promoter, which also drives expression of a plasmid copy of prfA. These genes are introduced into Gram-positive/Gram-negative bacteria shuttle plasmid pAM401, which can be amplified in E. coli as well as in Listeria since genetic manipulations cannot be readily carried out in Gram-positive organisms. Therefore plasmid genes include replication factors for Gram-positive and Gram-negative bacteria as well as antibiotic selection markers (chloramphenicol) for Gram-positive and Gram-negative bacteria. The plasmid confers resistance to chloramphenicol and is maintained in vitro by culture in the presence of chloramphenicol. In vivo, the plasmid is retained by trans complementation of the virulence factor PrfA, inactivated in XFL-7.

Described herein are cell-based assays, using differentiated TIP-1 cells, to analyze intracellular growth of Listeria based immunotherapies. TIP-1 cells are human monocytic cells that can be differentiated into macrophages by stimulating with Phorbol 12-myristate 13-acetate (PMA). Bacteria are quantitated pre-infection and post-infection at specific time points by lysis of TIP-1 cells and plating bacterial dilutions on brain heart infusion agar. Colony forming units (CFU) represent viable organisms surviving the macrophage intracellular environment.

Exemplary procedures using ADXS11-0001 is set forth below. However, these procedures and the procedures described in the other examples can be used for any Listeria strain. The sample(s) and reference standard are thawed, pelleted, re-suspended, and diluted to the target cell number prior to infection.

TABLE 5
Exemplary Materials.

Sterile Polypropylene Tubes, various sizes, 15 mL to 50 mL
(Falcon or equivalent)
Latex Gloves (Dynarex or equivalent)
24 well plates (Costar, Cat #3524, or equivalent)
3 mL syringes (BD, 309577, or equivalent)

| | | | |
|---|---|---|---|
| Sterile Serological | 1 | mL | Falcon 356520 or equivalent |
| pipettes, | 2 | mL | Falcon 357558 or equivalent |
| | 5 | mL | Fisher 1367610H or equivalent |
| | 10 | mL | Fisher 13676105 or equivalent |
| | 25 | mL | Fisher 1367811 or equivalent |
| | 50 | mL | Fisher 229230 or equivalent |
| | 100 | mL | Fisher 357600 or equivalent |

Pipette Barrier Tips (Avant or equivalent)
Plate Spreaders (Copan Diagnostics Cat #174C510, or equivalent)
Syringe and Needle (BD safetylock 1 mL #305554)
Sterile Microcentrifuge tubes, 1.7 mL, VWR 3620 or equivalent

| | | | |
|---|---|---|---|
| Centrifuge tubes | 15 | mL | Falcon 352059 or equivalent |
| | 50 | mL | VWR 352070 or equivalent |
| Pipette tips | 10 | μL | Fisher brand 02-777-155 or equivalent |
| | 20 | μL | Fisher brand 02-717-161 or equivalent |
| | 200 | μL | Fisher brand 2770 or equivalent |
| | 1 | mL | Fisher brand 02-717-166 or equivalent |
| Cuvettes | 1 | mL | Fisher 212371 or equivalent |
| | 1.5 | mL | VWR 7590750 or equivalent |
| | 2.5 | mL | VWR 7590700 or equivalent |

TABLE 6
Exemplary Apparatus/Equipment.
Hemocytometer (Bright Line, or equivalent)
Microscope (Olympus CK40 Inverted Microscope, or equivalent)
Laboratory Timer (VWR, 46610-060, or equivalent)
Centrifuge (Beckman Coulter, Allegra X-30R or equivalent)
Centrifuge (Eppendorf 5418, or equivalent)
Water Bath, 36 ± 2° C. (Shel lab, or equivalent)
Incubator, 36 ± 2° C., 5 ± 1% CO₂ (Lab line CO₂, or equivalent)
Storage Unit, 5 ± 3° C. (Kenmore, or equivalent)
Freezer, −20 ± 10° C. (Frigidaire, or equivalent)
Freezer, −80 ± 10° C. (Sanyo, or equivalent)
Incubator, 36 ± 2° C., (New Brunswick scientific, or equivalent)

| Pipette aid | Drummond scientific 156153 or equivalent | | |
|---|---|---|---|
| Pipette | 10 μL | VWR | 459020862 or equivalent |
| | 20 μL | VWR | 459030937 or equivalent |
| | 200 μL | VWR | 459051087 or equivalent |
| | 1000 μL | VWR | 459061892 or equivalent |

### Chemicals/Reagents

Prior to using in this assay, BHI plates can be visually inspected to ensure no gross contamination and for even spread of agar. Plates can be checked for growth suitability by streaking with wild type 10403S and ADXS11-001 and incubating at 37° C. for 24 hours. Colonies should be visible for both wild type and ADXS11-001.

TABLE 7
Reagents.

RPMI 1640 (Sigma, Cat# R8758 or equivalent)
FBS (Sigma, Cat# F0926 or equivalent)
L-glutamine 200 mM (Cellgro, Cat# 25-005-CL or equivalent)
Phorbol 12-myristate 13-acetate (PMA),
Sigma, Cat# P8139 or equivalent
DMSO (Amresco, Cat# 67-68-5 or equivalent)
10 mg/mL Gentamicin (Sigma, Cat# 221465, or equivalent)
25 mg/mL Chloramphenicol (Amresco (VWR),
Cat# 56-75-7, or equivalent)
Brain Heart Infusion Agar plates
(BD, Cat# PA-255003.08, or equivalent)
PBS- Calcium and Magnesium free
(Fisher, Cat# 10010-023, or equivalent)
Sterile water (WFI) (Fisher, Cat# BP2470-1 or equivalent)
Wild type: Listeria monocytogenes (Lm) (PHE Culture Collections)
THP1 cell line: Sigma, Cat# 88081201
Streptomycin, 100 mg/mL (Sigma-Aldrich 56501 or equivalent)
Sterile water for injection, Cat# BP281-1 or equivalent
Current ADXS11-001 Reference standard

Reagent Preparation. All reagent preparations can be adjusted to meet the required volumes needed.

Complete RPMI (c-RPMI)
1. To 445 mL of RPMI, add the following: (1) 50 mL of FBS—irradiated; (2) 5 mL of L-glutamine (200 mM).
2. Label and store at 5±3° C. Expiration is 1 month from the preparation date.

### 1.6 mM PMA (Phorbol-12-Myristate-13-Acetate)

1. Reconstitute 1 mg of PMA in 1 mL of DMSO for a final concentration of 1.6 mM PMA.
2. Aliquot 10 L into microcentrifuge tubes until depleted.
3. Label and store at −20±10° C. Expiration is 6 months from the preparation date.
   25 μg/mL Chloramphenicol
1. Reconstitute 0.5 g of Chloramphenicol in 20 mL of 100% Ethanol for a final concentration of 25 μg/mL Chloramphenicol.
2. Label and store at −20±10° C. Expiration is 1 month from the preparation date.
   100 μg/mL Streptomycin
1. Reconstitute 4 g of Streptomycin in 40 mL of sterile water to a final concentration of 100 g/mL Streptomycin. Sterilize using 0.2 micron filter. Aliquot 1 mL into 1.5 mL Tubes until depleted.
2. Label and store at −20±10° C. Expiration is 1 month from the date of preparation.
   Brain Heart Infusion Agar+25 μg/mL Chloramphenicol
1. Verify that BHI plates have no manufacturing defects (contaminations, broken plates, uneven agar etc.) before proceeding.
2. Add 180 μL of sterile PBS and 20 μL of Chloramphenicol (25 mg/mL) to Brain Heart Infusion agar plate and spread using sterile spreader to cover the entire surface of the plate. Spread until all the liquid is absorbed by the agar plate.
3. If preparing more than one plate, working stock of Chloramphenicol can be prepared (180 μL×no. of plates for PBS and 20 μL×no. of plates for Chloramphenicol) and 200 μL added to each plate and spread using sterile spreader.
4. The expiration date of the BHI+25 μg/mL Chloramphenicol plate will be either the expiration date of the plate as per the manufacturer or the expiration date of Chloramphenicol stock whichever is the earliest.
   Brain Heart Infusion Agar+100 μg/mL Streptomycin
1. Verify that BHI plates have no manufacturing defects (contaminations, broken plates, uneven agar etc.) before proceeding.
2. Add 180 μL of sterile PBS and 20 μL of Streptomycin (100 mg/mL) to Brain Heart Infusion agar plate and spread using sterile spreader to cover the entire surface of the plate. Spread until all the liquid is absorbed by the agar plate.
3. If preparing more than one plate, working stock of Streptomycin can be prepared (180 μL×no. of plates for PBS and 20 μL×no. of plates for Streptomycin) and 200 μL added to each plate and spread using sterile spreader.
4. The expiration date of the BHI+100 μg/mL Streptomycin plate will be either the expiration date of the plate as per the manufacturer or the expiration date of Streptomycin stock, whichever is the earliest.

Controls

### Negative/Sterility Controls

1. Uninoculated: Three plates of each, BHI Agar+Streptomycin 100 μg/mL and BHI Agar+25 μg/mL Chloramphenicol.
2. Inoculated: Three plates of each, BHI Agar+Streptomycin 100 μg/mL and BHI Agar+25 μg/mL Chloramphenicol each inoculated with 100 μL of PBS

### Positive Controls

1. Wild type: Two plates streaked with Listeria monocytogenes (PHE Culture Collections 10403S) on BHI Agar+Streptomycin 100 μg/mL.
2. ADXS11-001: Two plates streaked with ADXS11-001 reference standard will act as a positive control on BHI Agar+25 μg/mL Chloramphenicol

Preparation of THP-1 Cells
1. Thaw sufficient vials of THP-1 cells as required for procedure step 3.
2. Subpassage at least twice post thaw. In some embodiments, the THP-1 cells are below passage number of 32.
3. THP-1 cells already in culture can be used for the assay with the appropriate cell culture reference.
4. Prepare 40 mL of cells at a concentration of 1.0×10⁶ cells/mL in c-RPMI. Determine cell count.
5. Add 1 mL of cell suspension to each of two wells on a 24-well plate (see Plate Map in Appendix 2, Table 9 for one example). Label wells as “No PMA.” To the remaining cell suspension (approximately 34 mL), add 16 μM PMA (34 μL) for a final concentration of approximately 16 nM PMA. Mix well.
6. Distribute 1 mL per well for a total of 10 wells (see Plate Map in Appendix 2, Table 9, for example).
7. Incubate overnight (16-20 h) at 36±1° C., 5±1% CO₂.

Infection. The following steps 1-13 are performed for the positive control, wild type bacteria and will be repeated for the reference standard and sample bacteria.
1. Retrieve one vial of Positive Control wild type L. monocytogenes or reference standard or sample L. monocytogenes as appropriate.
2. To thaw the vial completely, incubate at 36±2° C. for 1 minute followed by incubation at room temperature for up to 5 minutes.
3. Transfer total volume to a respectively labeled 1.5 mL centrifuge tube with syringe and needle.
4. Transfer 1.0 mL to a respectively labeled 1.5 mL centrifuge tubes. Discard residual material.
5. Pellet 1.0 mL of cells at 16,100×g using a microcentrifuge for 2 minutes. Discard supernatant and resuspend cells with 1.0 mL of room temperature c-RPMI. Prepare dilutions of bacteria using c-RPMI for a final concentration of 1.0×10⁶ CFU/mL. The final volume at this concentration should be approximately 15 mL.
6. Obtain a 24 well plate containing THP-1 cells. Label wells as wild type or with sample number as appropriate.
7. Observe cells under microscope and confirm distinction between cells treated with PMA and those untreated. Untreated cells will exhibit fluidity when shaken lightly. Treated cells will remain adherent when shaken lightly.
8. Aspirate media from all wells containing PMA treated cells using a pipette (vacuum aid can be used).
9. Transfer 1.0 mL of the prepared bacteria (step 6; 1×10⁶ CFU/mL) to wells of the plate.
10. Observe plates on a microscope to ensure that THP-1 cells are still adhering to the surface of the wells.
11. Incubate plate at 36±2° C., 5±1% CO₂. Record incubation start time. Plate is incubated for 2 hours before its next manipulation.
12. Perform viability testing of the 1×10⁶ CFU/mL dilution of bacteria, designated p-2. Utilize procedure outlined in Appendix 1. Utilize dilution scheme outlined in Appendix 1. Prepare positive and negative controls outlined in Appendix 1.
13. Repeat steps 1-12 using test L. monocytogenes sample (e.g., ADXS11-001).

Infection Stop. The following steps 1-10 will first be performed for the positive control, wild type bacteria and will be repeated for the sample bacteria.
1. Prepare c-RPMI containing 20 μg/mL gentamicin. 2. After 2 hours, remove plate containing wild type or sample from 36±2° C., 5±1% CO₂ incubating conditions.
3. Remove L. monocytogenes containing media from each well using a pipette (vacuum aid can be used).
4. Carefully dispense 1 mL per well of the prepared c-RPMI containing 20 μg/mL gentamicin, with slow addition against the side of the well to avoid disruption.
5. Return plate to incubating conditions (36±2° C., 5±1% CO₂) for 42-45 minutes.
6. Remove plate from incubating conditions.
7. Remove c-RPMI containing 20 μg/mL gentamicin from each well using a pipette (vacuum aid can be used).
8. Wash cells carefully by addition of 1 mL of c-RPMI without gentamicin per well, slowly adding against the side of well to avoid disruption.
9. Remove c-RPMI from each well using a pipette (vacuum aid can be used).
10. Carefully dispense 1 mL of c-RPMI (without gentamicin) to each well by slowly adding against the side of well to avoid disruption and return plate to incubating conditions, 36±2° C., 5±1% CO₂ for 5-15 minutes.
11. Repeat steps 1-10 using plate containing reference standard and repeat using plate containing sample (e.g., ADXS11-001).

Collection Procedure for Detection of Intracellular L. monocytogenes growth
1. Remove plate from incubating conditions and record the time. First time point will be p0. Subsequent time points will be taken at p3 (3 hours) and optionally p5 (5 hours).
2. Observe wells under a microscope. Confirm that the layer of PMA treated THP-1 cells in each well is consistent and that minimal to no cells were dislodged and removed during the previous aspiration and dispensing steps. If any wells are observed to have significant THP-1 cell loss, mark well with an “X” to know not to use it.
3. Pick one well to use for the time point “p0” collection.
4. Remove the c-RPMI from the selected well by aspiration with a pipette (vacuum aid can be used).
5. Dispense 1 mL of sterile water into the well and using a micropipette, dislodge the THP-1 cells from the surface of the well by pipetting up and down.
6. Transfer entire contents into a 1.5 mL centrifuge tube.
7. Observe well under a microscope to confirm that cells have successfully been removed. If a significant portion of THP-1 cells remain, utilize a portion of the sterile water previously transferred to a 1.5 mL tube to dislodge the cells by pipetting up and down. Transfer contents back into 1.5 mL tube and confirm via microscopy that THP-1 cells have been removed.
8. Return plate to incubating conditions (36±2° C., 5±1% CO₂) until the next time point is ready to be collected.
9. Vortex the tube for at least 1 minute.
10. Perform viability testing. Utilize procedure outlined in Appendix 1. Utilize Dilution scheme outlined in Appendix 1, Table 8.
11. Repeat steps 1-10 using plate containing reference standard and repeat using plate containing sample (e.g., ADXS11-001).

Calculations
1. Uptake (p-2/p0) as a ratio of sample to wild type.
2. Intracellular Growth (p3/p0) as a ratio of wild type to sample.

APPENDIX 1—Viability testing procedure
1. Ensure that all agar plates are sufficiently dry prior to initiation of viability testing.
2. Prepare the following negative controls: (1) three un-inoculated plates of the appropriate agar type; and (2) three plates of the appropriate agar type inoculated with 100 μL of PBS and spread with a sterile spreader.
3. Vortex 1.0 mL aliquot of THP-1 cells at max speed for 60 seconds. The p-2 time point viability will instead utilize the 1.0×10⁶ CFU/mL dilution prepared in PBS.
4. Serial dilutions will be prepared based on the L. monocytogenes cell type (wild type or Sample) and the time point being tested. Refer to Table 8. Serial dilutions prepared by transferring 100 μL of the vortexed 1.0 mL aliquot into 900 μL of PBS. This process is repeated until all dilutions required are obtained.
5. The inoculum for each dilution will be spread with a sterile spreader onto the appropriate agar type in triplicate.
6. Prepare the following positive controls. The appropriate positive control will be inoculated onto the appropriate agar, in duplicate, with 10 μL inoculating loops.
7. Each plate will be allowed to absorb liquid and dry with its lid on for at least 15 minutes before being inverted and placed in an incubator at 35-38° C.
8. After 16-24 hours, remove plates from incubating conditions. Ensure that all Listeria monocytogenes cell types (wild type and Sample) at each time point are incubated for the same duration.
9. Total number of colony forming units (CFUs) will be counted manually and recorded for each plate of each dilution.

TABLE 8
Dilutions to Be Used for Viability Testing of Wild Type and
Sample at Each Time Point (Values May Be Adjusted as Needed).

| Construct | Time Points | Dilutions to titrate |
|---|---|---|
| Wild Type Lm | p-2 | 10¹, 10², 10³ |
| | p0 | 10¹, 10², 10³ |
| | p3 | 10¹, 10², 10³ |
| ADXS11-001 Drug Product | p-2 | 10¹, 10², 10³ |
| Reference standard or | p0 | 10¹, 10², 10³ |
| Sample | p3 | 10¹, 10², 10³ |
| ADXS11-001 Drug Product | p-2 | 10¹, 10², 10³ |
| Sample | p0 | 10¹, 10², 10³ |
| | p3 | 10¹, 10², 10³ |

APPENDIX 2—Preparation of 24-Well Plates. Preparation of the 24-well plates is shown below. Perform this plate setup for the Lm wild type then repeat for the reference standard and for the sample (e.g., ADXS11-001). Plate wells set up can be adjusted based on number of TIP-1 cells counted at time of seeding and the time points to be tested.

TABLE 9
24-Well Plate Set-Up.

| | 1 | 2 | 3 | 4 | 5 | 6 |
|---|---|---|---|---|---|---|
| A | THP-1 Cells | THP-1 Cells | THP-1 Cells | THP-1 Cells | THP-1 Cells | THP-1 |
| | with 16 nM | with 16 nM | with 16 nM | with 16 nM | with 16 nM | Cells |
| | PMA Final | PMA Final | PMA Final | PMA Final | PMA Final | NO PMA |
| | Concentration | Concentration | Concentration | Concentration | Concentration | |
| B | THP-1 Cells | THP-1 Cells | THP-1 Cells | THP-1 Cells | THP-1 Cells | THP-1 |
| | with 16 nM | with 16 nM | with 16 nM | with 16 nM | with 16 nM | Cells |
| | PMA Final | PMA Final | PMA Final | PMA Final | PMA Final | NO PMA |
| | Concentration | Concentration | Concentration | Concentration | Concentration | |
| C | Empty | Empty | Empty | Empty | Empty | Empty |
| D | Empty | Empty | Empty | Empty | Empty | Empty |

### Example 2. Validation of THP-1-Based Assays for Quantifying Intracellular Growth of Listeria monocytogenes

This qualification study was conducted to demonstrate that the method described in Example 1 could be used to quantify attenuation of ADXS11-001 Drug Product compared to wild type Listeria monocytogenes (Lm). The method utilized human THP-1 cells and assessed the uptake and intracellular growth of ADXS11-001 Drug Product or wild type Lm in the TP-1 cells. This example summarizes the data generated from qualification experiments.

TABLE 10
Summary—Method Qualification Table.

| Parameter | Results |
|---|---|
| Precision | 20% Relative Standard Deviation (RSD) Max—Wild type |
| (Intra-Assay Repeatability) | 21% RSD Max—ADXS11-001 reference Standard |
| | No significant difference in Rate of growth |
| Intermediate Precision | 47% RSD Max—Wild type viable cell counts |
| | (VCC) per time point |
| (Inter-Assay Repeatability) | 23% RSD Max—ADXS11-001 VCC per time point |
| | 29% RSD Max—Reportable Value (p3/p0) |
| Specificity | No growth in Negative controls |
| | Intracellular growth observed |

Listeria monocytogenes is the Gram positive, non-spore forming bacterial organism that exhibits unique life-cycle in an antigen-presenting cell (APC). After initial uptake of Lm by APC phagosome, where the expression of cytolysin, Listeriolysin O (tLLO) is triggered, that mediates the escape of Lm from phagosomes. Once escaped, Lm is able to replicate intracellularly within the cytosol of its host. A cell-based assay, using differentiated TIP-1 cells, was used to analyze uptake and intracellular growth of Listeria based vaccines. TIP-1 cells are human macrophage cells, maintained in culture as monocytes but can easily be differentiated into macrophages by stimulating with Phorbol 12-myristate 13-acetate (PMA). Bacteria are quantitated pre- and post-infection at specific time points by lysis of TIP-1 cells and plating bacterial dilutions on brain heart infusion agar. Colony forming units (CFU) represent viable Lm surviving the macrophage intracellular environment.

The strain ADXS11-001 contains a plasmid for the expression of the protein of interest (i.e., human papillomavirus protein E7 fused to truncated Listeriolysin O (tLLO)). The TIP-1 infection assay was used to demonstrate attenuation of ADXS11-001 with respect to wild type parent strain 10403S. In this assay, TP1 cells were infected with either 10403S or ADXS11-001 at multiplicity of infection of 1:1, and in vitro growth of bacterial CFU was analyzed at different time points such as 1 h, 3 h and 5 h post-infection. As a result of attenuation, a significant reduction in the uptake and intracellular growth of ADXS11-001 was observed compared to 10403S.

Control Preparation. Wild type Lm 10403S and ADXS11-001 DP were prepared as described in Example 1. Briefly, samples were thawed at 36±2° C. and centrifuged, and concentration was adjusted to 1.0×10⁶ cells/mL using complete RPMI.

THP-1 Cells Preparation. THP-1 cell bank, Passage number P33, was frozen at a density of 1×10⁶ viable cells/mL. The TIP-1 cells; P33, were prepared as described in Example 1. Briefly, TIP-1 cells were plated at a concentration of 1.0×10⁶ cells/mL/well in 24 well plate in complete RPMI containing 16 nM PMA.

Sample Preparation. PMA-differentiated TIP-1 cells were infected with wild type Lm 10403S and ADXS11-001 DP as in Example 1. Bacterial colony forming units (CFU) were quantitated pre- and post-infection at specific time points by lysis of TIP-1 cells and by plating bacterial dilutions on agar plates.

Results. Results were generated from three independent experiments. The CFUs generated from each dilution and each time point from controls and samples were analyzed to capture all required calculations and qualification parameters were evaluated. Calculations of means, standard deviations, coefficients of variation, and raw data outputs were determined for inter- and intra-assay precision, and specificity was evaluated for each run.

Viability expressed as the number of cells counted serves as the raw data output of this assay. The following criteria was used for determination of viability. Data from an assay were considered acceptable only if the negative controls (un-inoculated and PBS inoculated plates) showed no colony growth. Colony forming unit (CFU) less than 40 was considered Too Few To Count (TFTC) and CFU greater than 600 was considered Too Numerous To Count (TNTC). Only values within these limits were quantified.

### Precision (Intra-Assay Repeatability)

The % Relative Standard Deviation (RSD) for values of replicate controls and samples were calculated for intra-assay precision. % RSD for triplicate wells in each of the three assays at each time point ranged from 11% to 20% for the wild type and 9% to 21% for the ADXS11-001 reference standard sample. The maximum intra-assay variation as measured by % RSD across all time points for both the wild type and the ADXS11-001 reference standard was 21% and was observed at the p1 time point. p1 values were not however used in calculating reportable results. Intra-assay precession is expected to be well within the 21% RSD.

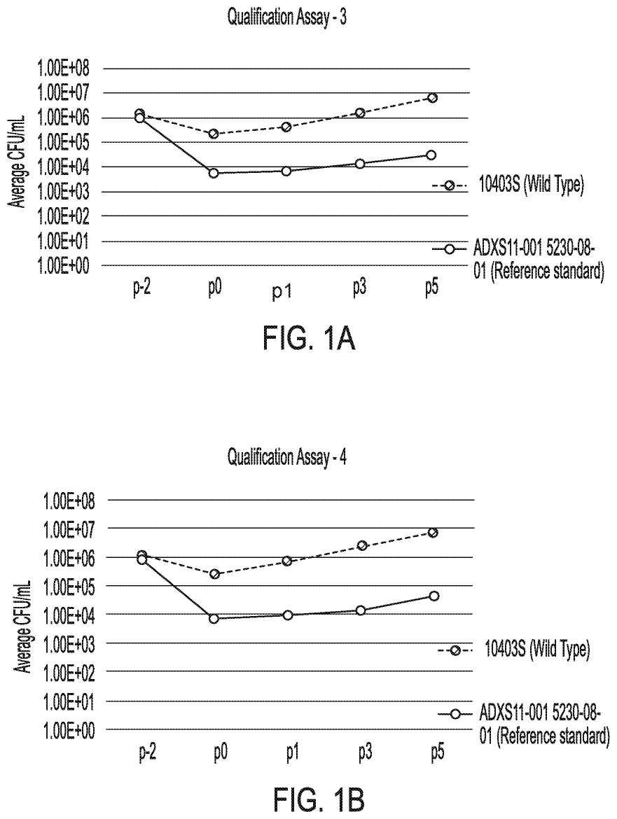

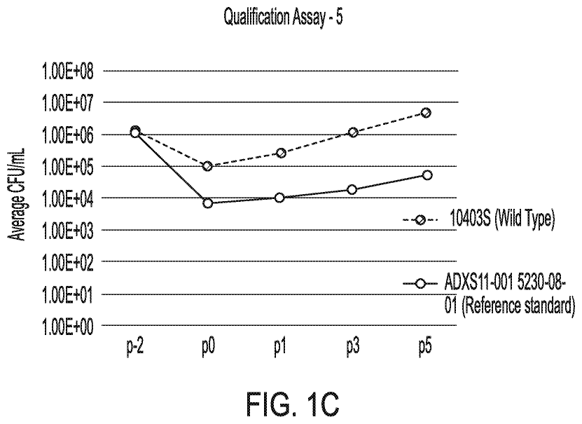

Values for p0, used in calculating the reportable value for the assay were 20% RSD Max for the wild type and 9% RSD Max for the Reference standard. Intracellular growth outputs, p3 and p5, showed Max % RSDs of 11% and 17% respectively for the wild type and 10% and 19% for the ADXS11-001 reference standard. p3 values showed greater inter-assay precision than p5 values. RSD Values for intra-assay precision are summarized in Table 11. Additionally, the rate of growth as demonstrated in the curves in FIG. 1, plotted as time versus viable cell counts (VCC) showed no apparent difference between wells as all curves show the same general shape and trend.

TABLE 11
% RSD Values at Each Time Point for
Each of the Qualification Assays.

| | | Assay # | | | | |
|---|---|---|---|---|---|---|
| | Time point | 3 | 4 | 5 | Average | Max |
| Wild Type | p-2 | 15 | 2 | 6 | 8 | 15 |
| | p0 | 16 | 20 | 9 | 15 | 20 |
| | p1 | 6 | 14 | 3 | 8 | 14 |
| | p3 | 11 | 4 | 3 | 6 | 11 |
| | p5 | 3 | 17 | 10 | 10 | 17 |
| ADXS11-001 | p-2 | 12 | 8 | 8 | 9 | 12 |
| Reference standard | p0 | 5 | 9 | 4 | 6 | 9 |
| | p1 | 21 | 12 | 7 | 13 | 21 |
| | p3 | 10 | 7 | 8 | 8 | 10 |
| | p5 | 13 | 19 | 12 | 15 | 19 |

### Intermediate Precision.

Values obtained for three independent assays performed on multiple days by at two analysts were used to evaluate intermediate precision. Three assays utilizing three THP-1 cell passage numbers and infection and titration were done. The degree of agreement between individual test results expressed as the coefficient of variation including agreement between the averages of three replicate measurements of the sample at each post infection time point from each independent assay preparation was assessed. The assessment also included the agreement between the averages of three replicate measurements of the wild type control at each post infection time point from each independent assay preparation.

(A) Raw data, VCC at each time point. The VCC values normalized for dilution at each time point were calculated for all three assays. The highest % RSD observed for the wild type was 47% and for the ADXS11-001 reference standard was 23%. Results are summarized in Table 12. It should be noted that raw data % RSDs are not as significant since for the reportable results these values are further transformed into a ratio.

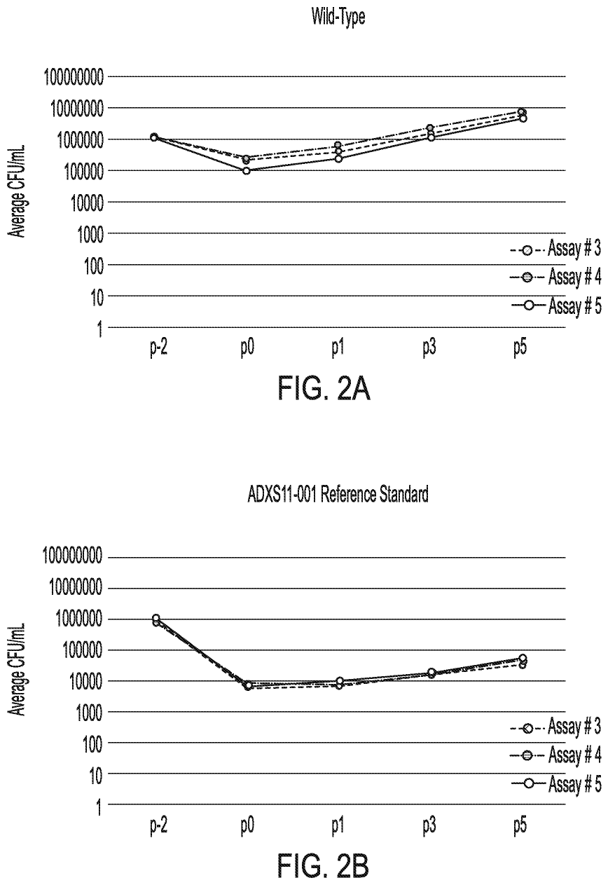

In addition, the rate of growth as demonstrated in the curves in FIG. 2, plotted as time versus viable cell counts (VCC) showed no apparent difference between the assays as all curves show the same general shape and trend.

TABLE 12
Raw Data VCCs Normalized for Dilution for Each Time
Point for Each of the Qualification Assays.

| | | Assay # | | | | |
|---|---|---|---|---|---|---|
| | | 3 | 4 | 5 | | |
| | | A1 titration | A1 titration | A2 titration | | |
| | Time point | A2 Infection | A2 Infection | A1 Infection | Average | % RSD |
| Wild Type | p-2 | 1300000 | 1236667 | 1333333 | 1290000 | 4% |
| | p0 | 248667 | 282667 | 106333 | 212556 | 44% |
| | p1 | 481167 | 734667 | 269000 | 494944 | 47% |
| | p3 | 1776667 | 2703333 | 1196667 | 1892222 | 40% |
| | p5 | 7166667 | 8633333 | 4853333 | 6884444 | 28% |
| ADXS11-001 | p-2 | 870000 | 900000 | 1080000 | 950000 | 12% |
| Reference | p0 | 5967 | 8100 | 7133 | 7067 | 15% |
| standard | p1 | 7500 | 10500 | 10300 | 9433 | 18% |
| | p3 | 16067 | 16400 | 19233 | 17233 | 10% |
| | p5 | 33700 | 51267 | 53150 | 46039 | 23% |

A1 = Analyst 1
A2 = Analyst 2

(B) Assay ratios (reportable values). For each experiment the reportable values were calculated as follows:

$$Uptake for Sample \text{:} Ratio at time point p 0 = \frac{{[ VCC ( p - 2 ) VCC ( p 0 ) ]}^{sample}}{{[ VC C ( p - 2 ) N C C ) p 0 ) ]}^{Wild type}} \\$$

$$\text{Intracellular Growth} : Ratio at time point p 3 = \frac{{[ VCC ( p 3 ) VCC ( p 0 ) ]}^{Wild type}}{{[ VCC ( p 3 ) NCC ( p 0 ) ]}^{sample}}$$

$$\text{Intracellular Growth} : Ratio at time point p 5 = \frac{{[ VCC ( p 5 ) VCC ( p 0 ) ]}^{Wild type}}{{[ VC C ( p 5 ) V C C ( p 0 ) ]}^{sample}}$$

For all three assays the ratios were greater than 10 for sample uptake and greater than 2 for intracellular growth. The maximum % RSD was 39% between all three assays for sample uptake and a maximum of 29% for intracellular growth which is the reportable ratio value. Results are shown in Table 13. In addition, the rate of growth as demonstrated in the curves in FIG. 1, plotted as time versus viable cell counts (VCC) showed no apparent difference between the assays as all curves show the same general shape and trend.

TABLE 13
Reportable Values Results from the Three Qualification Assays.

| | | | Assay # | | | |
|---|---|---|---|---|---|---|
| | | 3 | 4 | 5 | | |
| | | A1 titration | A1 titration | A2 titration | | |
| Reportable value | | A2 Infection | A2 Infection | A1 Infection | Average | % RSD |
| Uptake for Sample: | p-2/p0 | 28 | 25 | 12 | 22 | 39% |
| Ratio at time | | | | | | |
| point p0 | | | | | | |
| Intracellular Growth: | p3/p0 | 3 | 5 | 4 | 4 | 29% |
| Ratio at time | | | | | | |
| point p3 | | | | | | |
| Intracellular Growth: | p5/p0 | 5 | 5 | 6 | 5 | 11% |
| Ratio at time | | | | | | |
| point p3 | | | | | | |

A1 = Analyst 1
A2 = Analyst 2

(C) Analyst. The infection portion of the assay for assays 3 and 4 were done by analyst 1 and by analysts 2 in assay 5. The titration portion of the assay for assays 3 and 4 were done by analyst 2 and by analysts 1 in assay 5. The data suggest there may be a possible difference in the wild type raw data values between analysts and this is reflected in the p-2/p0 ratio. Reportable values, p3/p0 and p5/p0 show no analyst effect. Assay ratios independent of analysts, were 12 or greater for uptake and greater than 3 for intracellular growth which is a sufficient enough fold difference to distinguish between the wild type strain and the ADXS11-001 reference standard or sample. See Table 13.

(D) Day. No significant effects were observed for the reportable values for assays performed over different days. Reportable values for Assay 3 and Assay 4 in which the titration and infection portions of the assays were performed by the same analysts were within 3 units for uptake (p-2/p0) and 2 units for intracellular growth. All values were 12 or greater for uptake and greater than 2 for intracellular growth which is a significant enough fold difference to distinguish between the wild type strain and the ADXS11-001 reference standard and sample. See Table 13.

(E) THP-1 cell passage number. The cell passage number showed no significant impact on the reportable values of the assay. THP1 cell passage P32, P37 and P39 were used in this qualification. Each passage number gave reportable values that were 12 or greater for uptake and greater than 2 for intracellular growth which is a significant enough fold difference to distinguish between the wild type strain and the ADXS11-001 reference standard and sample. See Table 13.

### Specificity

Un-inoculated and PBS inoculated blank samples of the TIP-1 matrix were tested for interference, and selectivity. This was included in each assay and no growth (no CFUs) were observed for blanks in all assays. These negative controls also demonstrate the absence of contamination which is indicative of no false negative or false positive results.

Detectable CFUs from the lysed THP-1 matrix in both sample and wild type control were generated in each of the assays. CFUs were detected from each of the assays in the presence of the same TP-1 matrix for the reference standard samples (ADXS11-001) and controls (wild type) thus demonstrating acceptable specificity.

Additionally, intracellular growth is an indicator that the recombinant Lm vaccine strain is able to enter the cells and multiply therefore supporting selectivity. Intracellular growth was observed and calculated for each of the three assays sufficient enough to demonstrate fold difference between the wild type strain and the ADXS11-001 reference standard and sample.

Results. The protocol set forth in Example 1 has been qualified for the analysis of Listeria monocytogenes infection and replication in differentiated TIP-1 cells for ADXS11-001. The method was demonstrated to be specific, in that the method detected a fold difference in the uptake and intracellular growth between the wild type and ADXS11-001. The method was also shown to be precise and repeatable, and reportable assay results were similarly independent of analyst, days on which the assays were performed, or TIP-1 cell passage number.

### Example 3. Optimization of THP-1-Based Assays for Quantifying Intracellular Growth of Listeria monocytogenes

Data were obtained from a total of 13 representative test runs using the method set forth in Example 1. The data were evaluated to look for improvements in method efficiency while maintaining key quality attributes, including determining whether a shorter time frame for the development of the response is reasonable (3 hours vs. 5 hours), finding an upper bound on the number of passages of the TIP-1 cells, finding a lower bound on the baseline change in response from p-2 to p0, and determining the utility of the p time point.

The subject method is a cell based macrophage cell infection assay to assess infection and replication of ADXS11-001 as part of evaluating its attenuation. This is performed using both wild type (WT) L. monocytogenes cells, 10403S, and specific ADXS11-001 samples in parallel. This is a cell-based assay and uses bacteria that are quantified pre- and post-infection at specific time points by lysis of THP-1 cells, and plating bacterial dilutions on agar. Colony forming units (CFUs) represent the count of viable organisms surviving the macrophage intracellular environment. The ratio of the CFUs quantified at the different time points to themselves and to the WT presents the opportunity to quantify results.

As part of the infection step of the method, a 24-well plate is used to develop differential responses for both samples and WT, and then these are sampled and incubated to obtain a viable cell count. This viable cell count is referred to as p-2, as it precedes the infection start, and a 2 hour incubation time, after which measurements are taken again for the samples and the WT. The measurements after this 2 hour incubation are referred to as p0. Subsequent viable cell count measurements are also taken after 1 h (p1), 3 h (p3) and 5 h (p5) incubation times. The method reports: (a) update for sample (p-2/p0) as a ratio of sample to WT; and (b) intracellular growth (p3/p0) as a ratio of WT to sample. Data sources are set forth in Table 14.

TABLE 14
Data Used in the Analysis.

| | | | | ADXS11- | | | | |
|---|---|---|---|---|---|---|---|---|
| | | | Lm-10403S | 001# 2008 | ADXS11- | ADXS11- | ADXS11- | ADXS11- |
| | | Passages | 10403S | lot 5230- | 001# | 001# | 001# | 001# |
| Run | source | (P) | (Wild Type) | 08-01 RS | 2013 | 2014 | 2015-01 | 2015-02 |
| Sep. 10, 2015 | non-GMP Runs | 23 | X | X | X | | | |
| Sep. 11, 2015 | non-GMP Runs | 24 | X | X | X | | | |
| Sep. 16, 2015 | non-GMP Runs | 26 | X | | | X | X | X |
| Dec. 22, 2015 | Qualification Run | 32 | X | X | | | | |
| Jan. 22, 2016 | GMP Runs | 38 | X | X | | | | |
| Jan. 30, 2016 | Qualification Run | 39 | X | X | | | | |
| Feb. 2, 2016 | Qualification Run | 37 | X | X | | | | |
| Feb. 12, 2016 | GMP Runs | 38 | X | X | | | | |
| Feb. 25, 2016 | GMP Runs | 41 | X | X | | | | |
| Mar. 9, 2016 | Additional GMP | 34 | X | X | X | | | |
| | Runs | | | | | | | |
| Mar. 11, 2016 | Additional GMP | 34 | X | X | | X | | |
| | Runs | | | | | | | |
| Mar. 18, 2016 | Additional GMP | 36 | X | X | | | X | |
| | Runs | | | | | | | |
| Mar. 23, 2016 | Additional GMP | 38 | X | X | | | | X |
| | Runs | | | | | | | |

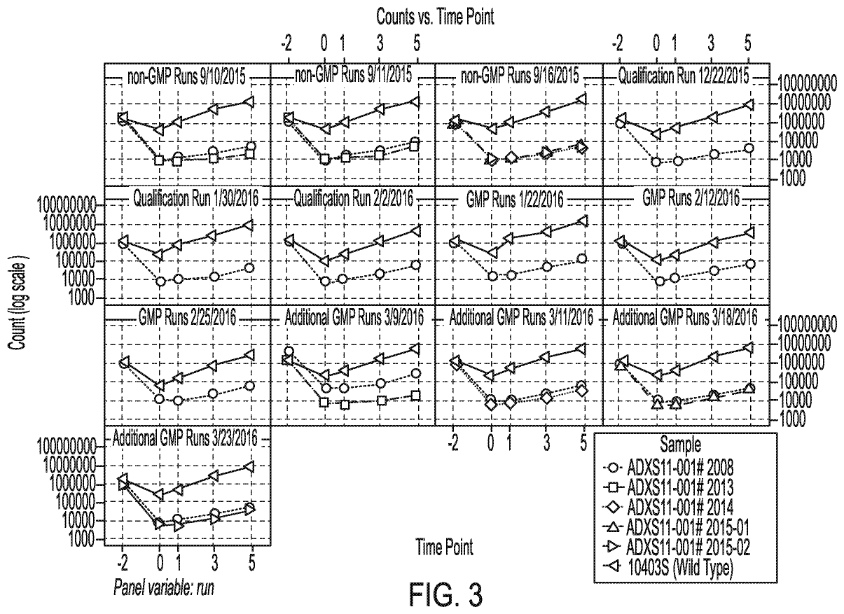

The results were screened for conformance to expectations. FIG. 3 displays the raw count information observed at all of the time points in the present method (p-2, p0, p1, p3, and p5). Each of the runs noted in Table 12 are in separate sub-plots, and the resulting curves for each of the batches in the key represent the test results. The data demonstrate an expected downward change in the first two hour period (p-2 to p0), followed by increases from p0 through p5.

Responses were as expected, with samples having notably lower counts after initial inoculation than the wild type organism, and similar rates of increase subsequent to the p0 time point.

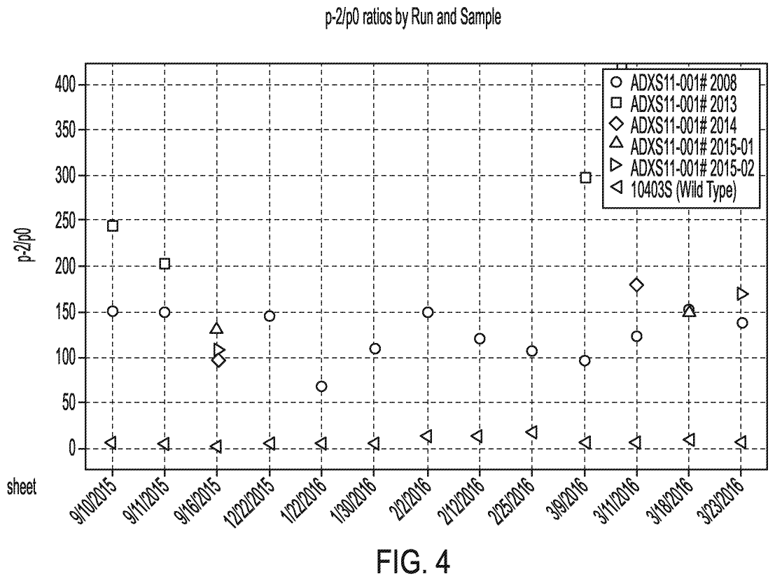

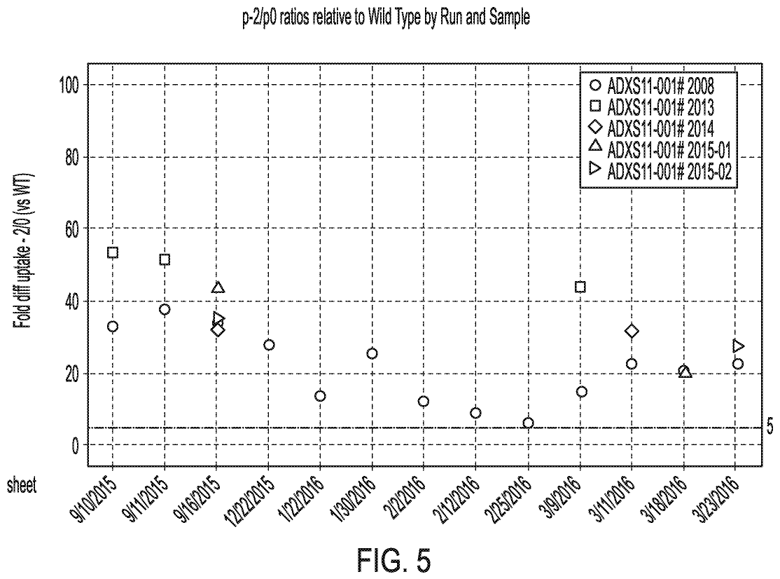

FIG. 4 and FIG. 5 show a graphic portrayal of the data for the uptake for sample growth relative to wild type (WT). FIG. 4 shows the raw data, as the ratio of the count at p-2 to that seen at p0. The amount of change from p-2 to p0 is markedly different for samples. FIG. 5 shows the same, but converts the sample results as a ratio to wild type.

FIG. 5 shows that the ratio of the change in p-2/p0 response for samples vs wild type changes from run to run. The change is typically greater than a 5 fold difference for the sample relative to the wild type. This is shown as a red dashed line in FIG. 5. The relative response of the sample to wild type is related to the number of passages of the TIP-1 cells (shown below).

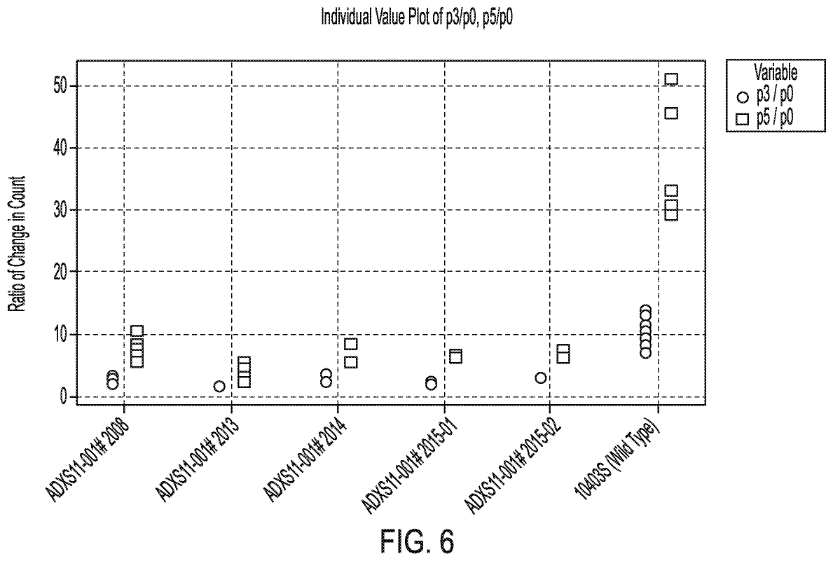

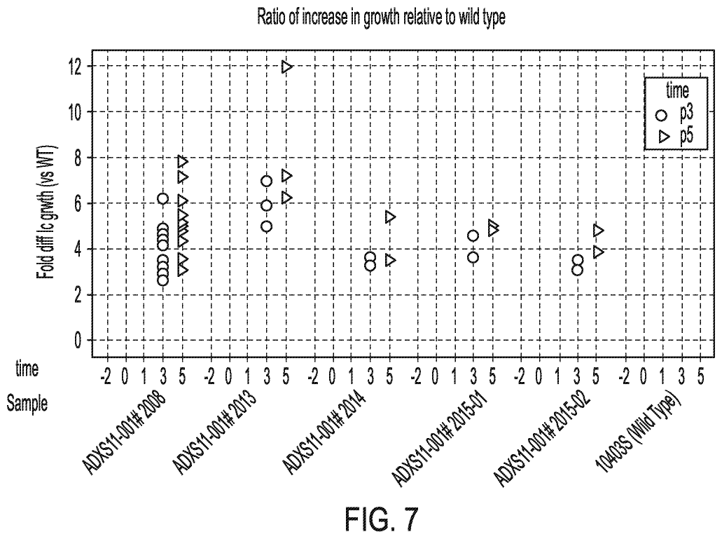

FIG. 6 and FIG. 7 show a graphic portrayal of the data for the intracellular growth (p3/p0) and (p5/p0) prior to taking ratio relative to wild type. FIG. 6 displays the ratios of samples prior to taking the ratio relative to wild type. There are notable differences in the results at p3 and p5 prior to taking the ratio. FIG. 7 adjusts the data as per the method to show the change in response relative to wild type. It demonstrates that the relative response at p3, p5 response is not substantially different. Similar variability is observed within sample type (run to run) and between samples.

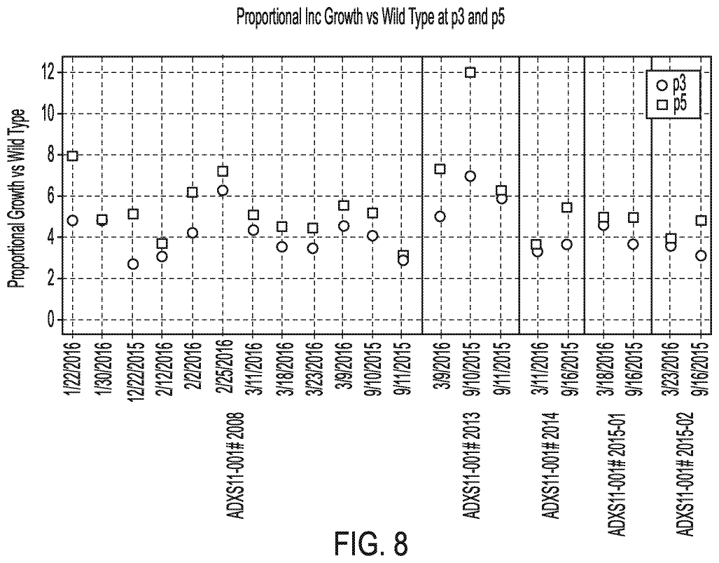

FIG. 8 plots the same result as shown in FIG. 7, but also breaks the data out by run. This view of the results shows that the differences in the ratio of growth at p3 and p5 are smaller than those seen from run to run within sample. The data support the use of the proportional growth at p3 vs wild type on this basis.

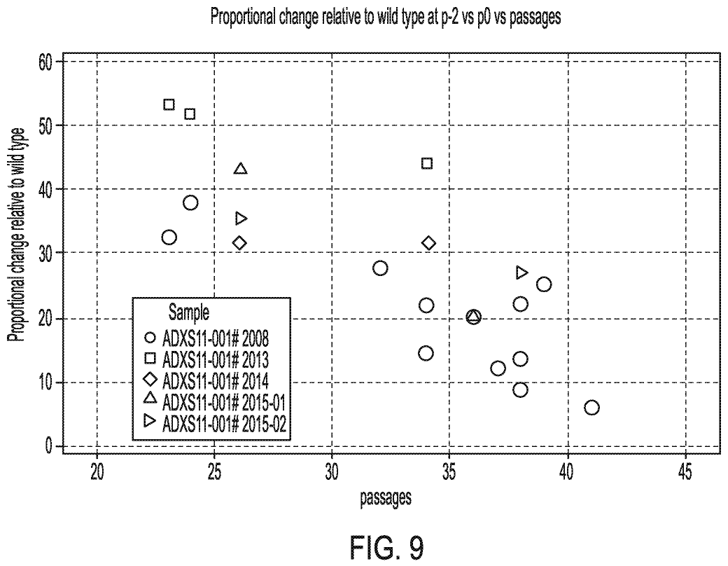

To evaluate the impact of the number of passages, the proportional decrease in counts from p-2 to p0 (relative to wild type) was plotted for each sample against the number of passages for the organisms in that run. FIG. 9 shows a clear relationship.

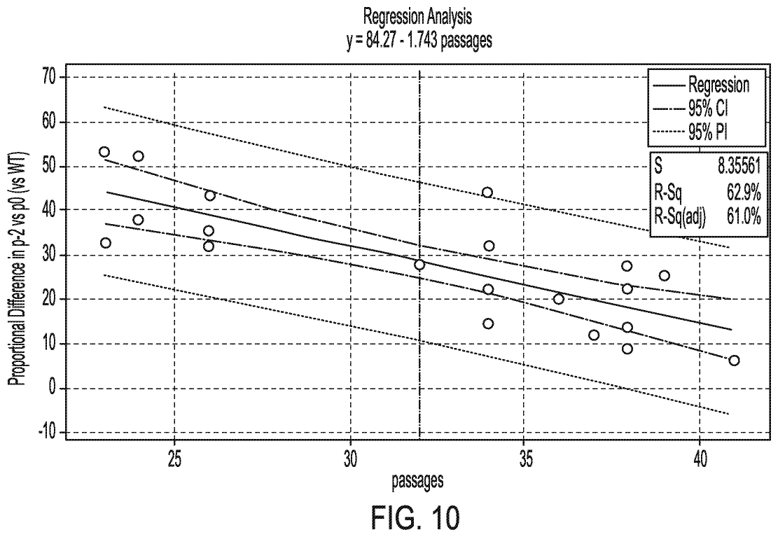

Based on this graph, a regression analysis was used to evaluate the impact of the number of passages quantitatively. Results are shown in FIG. 10. The regression equation demonstrates an approximate linear response, and indicates that at 32 passages, the 95% prediction interval for individual results is at a relative response of 10 (an order of magnitude difference). Based on this analysis, it is recommended that a maximum number of passages of 32 be used to assure that the relative response (proportional difference in the p0 results relative to p-2) remains above 10.

To establish the utility of the p1 time point, the following steps were taken for each of the individual curves in FIG. 3: (1) all counts were converted to a Log 10 scale; (2) using the responses at p0, p1 and p3 the slope was calculated; this represents the degree of change in count per hour when using all three time points; this is shown on the x-axis; and (3) the difference in the response at p3 and p0 was calculated, and divided by 3 to represent an hourly change; this is shown on the y-axis.

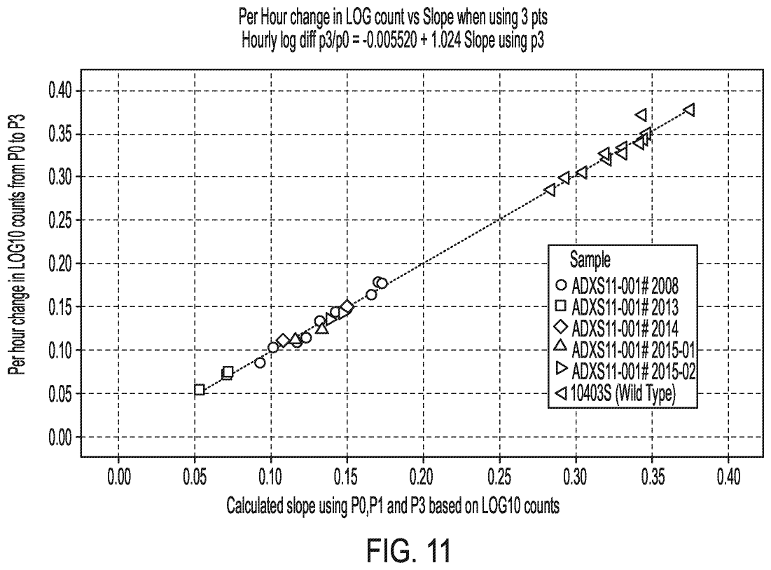

The relationship between the two resulting variables for each curve is plotted in FIG. 11. It shows that essentially the same value for the hourly change in Log 10 (count) is seen whether a simple difference is used or a slope is calculated using all three time points. The p1 time point is not essential for use in calculations.

Based on the evaluation of the data, the following are supported. p3 vs p0 can be employed in lieu of p5 vs p0 to evaluate the relative response of the sample vs wild type. The test can terminate at p3. The effect of passages can be significant, and applying an upper bound of 32 on the number of passages of the THP-1 may be recommended. Applying this upper bound to the number of passages will provide confidence that the baseline change in response from p-2 to p0 (wild type to sample) remains at least 10-fold, which is recommended as the lower bound. Results obtained at p1 are not essential for calculations of the degree of change for either samples or wild type.

### Example 4. THP-1-Based Infectivity Assays for Listeria monocytogenes

The ADXS11-001 is a cancer immunotherapy product which is a live attenuated Listeria monocytogenes strain genetically modified to express a fusion protein of listeriolysin O (LLO) and the Human Papilloma virus (HPV) protein E7, a tumor antigen found mainly in cells of cervical cancer, but also of vulvar, vaginal, penile and anal cancer as well as oropharyngeal cancer directly associated with Human Papilloma Virus 16 and 18, but also 31 and 45.

As a pathogen, Listeria monocytogenes is an intracellular pathogen infecting non-phagocytic and phagocytic cells by escaping into the cytoplasm after uptake into phagosomes. This is achieved by the expression of the protein listeriolysin O (LLO), which contributes to the disruption of the vacuolar membrane prior to fusion of the phagosome with lysosomes to form phagolysosomes. This allows the bacterium to escape into the cytoplasm, where it proliferates and spreads directly from cell to cell. THP-1 cells are a human macrophage cell line, maintained in culture as monocytes but can easily be differentiated into macrophage by stimulating with Phorbol 12-myristate 13-acetate (PMA).

The method described in this example is for determination of the Listeria monocytogenes drug product's (e.g., ADXS11-001) entry and escape into the cytoplasm at discrete time points post infection in differentiated THP-1 cells. PMA differentiated THP-1 cells are inoculated with wild type control and drug product ADXS11-001 respectively at 1:1 multiplicity of infection (M01). Infected THP-1 cells are then treated with gentamicin to kill extracellular bacteria. Bacteria are quantitated pre- and post-infection at specific time points by lysis of THP-1 cells and plating bacterial dilutions on Brain Heart Infusion (BHI) agar plates. Colony forming units (CFUs) represent viable organisms surviving the macrophage intracellular environment due to their escape from the lysosome.

An exemplary assay is set forth below. However, the assay can be used for any Listeria strain. Each assay occasion can evaluate up to 2 drug product samples against control along with reference standard.

TABLE 15
Assay Setup.

| Test | Time points | | | Number of dilutions | Number of agar |
|---|---|---|---|---|---|
| item | analyzed | | | per time point | plates per dilution |
| Control | p-2 | p0 | p3 | 3 (1:10; 1:100; 1:10000) | 3 |
| Reference | p-2 | p0 | p3 | 3 (1:10; 1:100; 1:10000) | 3 |
| standard | | | | | |
| Sample 1 | p-2 | p0 | p3 | 3 (1:10; 1:100; 1:10000) | 3 |
| Sample 2 | p-2 | p0 | p3 | 3 (1:10; 1:100; 1:10000) | 3 |

### Equipment, Reagents, and Consumables

Each assay occasion can evaluate up to 2 drug product samples against control along with reference standard.

TABLE 16
Equipment

| Equipment Class | Requirement |
|---|---|
| Biological safety cabinet | Class II |
| Incubator | 37 ± 1° C. & 5% ± 1 CO₂ setting |
| Incubator | 37 ± 1° C. Without CO₂ setting |
| Pipettes | 2-1000 μL |
| Water bath | To be set to 37° C. |
| | controlled by certified Thermometer |
| Cold storage | 2-8° C., −20° C., −70° C., −80° C. & LN₂ |
| Centrifuge | N/A |
| Microcentrifuge | 1.5/2.0 mL Eppendorf tubes, ~14,500 RCF |
| Vortex | N/A |

TABLE 17
Reagents.

| Reagent/consumable | Supplier | Catalogue code | Storage |
|---|---|---|---|
| Phorbol 12-myristate 13-acetate (PMA) | Sigma | P8139 | −20° C. |
| RPMI 1640 | Sigma | R0883 | 2-8° C. |
| Heat inactivated FBS | Biosera | FB-1001 | −20° C. |
| Gentamicin (50 mg/mL) | Thermo Fisher | 15750-060 | RT |
| PBS | GIBCO | 10010-015 | RT |
| Brain Heart Infusion agar plate (BHI) | Thermo Fisher | P01198A | 2-8° C. |
| Brain Heart Infusion agar plate (BHI) with 25 | Teknova | 81042 | 2-8° C. |
| pg/mL Chloramphenicol | | | |
| L-Glutamine | Sigma | G7513 | −20° C. |
| Streptomycin | Sigma | S6501 | 2-8° C. |
| Spreader | Gosselin | ETAR-06 | N/A |
| 24-well cell culture plates | Coming | CLS3524 | N/A |
| Sterile Water (for injection/irrigation) | Various | N/A | RT |
| DMSO | Sigma | D2650 | RT |
| Trypan Blue (0.4%) | Sigma | T8154 | RT |
| Serological pipettes—volumes as required | Various | Various | N/A |
| Sterile tubes—volumes as appropriate | Various | Various | N/A |
| Glycerol | Thermo | BP229-1/ | RT |
| | Fisher/Sigma | G2025 | |

### Reagent Preparation Instructions

Note: volumes/amounts can be scaled up ordown as required.

Complete RPMI for routine subculturing (c-RPMI) (500 mL): 445 mL RPMI 1640, 50 mL FBS, 5 mL L-glutamine (200 mM). Storage at 2-8° C. for up to 1 month.

Complete RRMI for thawing (c-RPMI-thaw) 505 mL: 400 mL RPMI 1640, 100 mL FBS, 5 mL L-glutamine (200 mM), stored at 2-8° C. for up to 1 month.

Freezing solution 500 mL: 450 mL Heat inactivated FBS, 50 mL glycerol, prepared fresh.

1.6 mM PMA: 1.0 mg PMA (Mw: 616.83), 1.0 mL DMSO, stored at −20° C. for up to 6 months. Prepare 10 μL aliquots in 2 mL sterile microcentrifuge tube. Each aliquot is single use.

100 mg/mL Streptomycin: 1 g Streptomycin, 10 mL sterile water, sterilized using a 0.2 μm filter, and stored at −20° C. for up to 1 month. Prepare 1 mL aliquots in 2 mL sterile microcentrifuge tube. Each aliquot is single use.

Brain Heart Infusion Agar+100 μg/mL Streptomycin. BHI plates were examiner to verify have no manufacturing defects (contaminations, broken plates, uneven agar etc.) before proceeding. The volume of each agar plate is approximately 22.8 mL. 177.2 ∝L×number of agar plates PBS, 22.8 ∝L×number of agar plates 100 mg/mL Streptomycin 100 mg/mL. 200 ∝L of diluted Streptomycin added to each agar plate. Spread using sterile spreader to cover the entire surface of the plate. Spread until all the liquid is absorbed by the agar plate. Plates are stored at 2-8° C. up to expiration date of either the agar plate or Streptomycin, whichever is earliest.

### THP-1 Cell Line Culturing

Thawing THP-1 cells. Perform procedure under aseptic conditions in a Biological Safety Cabinet. Only use materials that are certified sterile and prepared aseptically.
1. Pre-warm c-RPMI-thaw medium in water bath set to 37° C.
2. Place 3 mL of pre-warmed c-RPMI-thaw medium into a sterile 50 mL centrifuge tube.
3. Take THP-1 vial from cryogenic storage and thaw in a water bath set to 37° C. until the content is almost thawed, but a small amount of ice crystals remain in the tube.
4. Thoroughly clean the vial with disinfectant.
5. Add thawed cells drop by drop to 50 mL centrifuge tube containing 3 mL c-RPMI-thaw medium.
6. Wash the cryovial with additional 1 mL of c-RPMI-thaw medium and transfer to the 50 mL tube containing cells.
7. Take ˜100 μL cell suspension for counting.
   Note: to count using the hemocytometer dilute the cell suspension aliquot 1:2 in 0.4% Trypan blue. Ensure that suspension is well mixed by gentle pipetting. Use C-chips for counting. Conduct two independent counts. Determine cell viability (≥85%) and density.
8. Centrifuge cell suspension at 150×g for 5 minutes at RT
9. Discard the supernatant, re-suspend the cells in pre-warmed c-RPMI-thaw medium to give cell density of 1-3×10⁵ live cells/mL.
10. Transfer the contents of the tube to a cell culture flask (e.g. T75) and incubate at 37° C. 5% CO₂ absolute humidity.
11. Keep flask in a vertical position until the cells reach the exponential phase of growth.
12. Cells are normally counted every 2-3 days.

Note: Once the culture is established (normally 6 days after thawing), the serum concentration will be reduced to 10% by using c-RPMI medium.

Routine THP-1 cell culture. Procedures are performed under aseptic conditions in a Biological Safety Cabinet, using materials certified as sterile and prepared aseptically.

In some embodiments, for routine cell culture and THP-1 assay, cell passage number is limited to P32. Each transfer of cells to a new culture vessel is considered a passage. Addition of medium to the same culture vessel to assure exponential growth does not change the passage number.

To keep the cells in exponential growth, cultures are maintained between 3-8×10⁵ live cells/mL.
1. Inspect cells for morphology and contamination under microscope.
   Do not proceed if majority of cells is attached to the culture vessel surface. In such case discard the culture and thaw another vial of working cell bank.
2. Remove about 1 mL of cell suspension to a vial for total cell count and viability.
3. In order to keep cells in the exponential phase of growth, cells will be supplemented with fresh c-RPMI media to a density of 3×10⁵ cells/mL until the volume of cell suspension reaches maximum allowed volume, then cell suspension will be passaged into new pre-labelled flasks at a seeding density of 3×10⁵ cells/mL.
   Minimum and maximum volume range for different size of flasks are shown below for optimal CO₂ penetration: 775 flask: 15-37.5 mL; T150 flask: 30-75 mL
4. Incubate culture at 37° C., 5% CO₂ incubator.
5. Cells are normally counted every 2-3 days.

Cryopreservation of THP-1 cells. Procedures are performed under aseptic conditions in a Biological Safety Cabinet, using materials certified as sterile and prepared aseptically.
1. Follow “Routine THP-1 cell culture,” steps 1 to 2.
2. Prepare a freeze medium consisting of heat inactivated FBS supplemented with 10% (v/v) glycerol.
3. Centrifuge cells at 150×g for 5 minutes at RT.
4. Discard supernatant and resuspend the cells by tapping the tube until no clumps are visible. Slowly, drop-wise, add freeze medium by swirling the tube to give 2× final freezing density (final freezing density is 2×10⁶ cells/mL).
5. Slowly add a second, equal volume of freeze medium to the tube containing cells. Gently swirl the tube during the addition to allow complete mix.
6. Aliquot 1 mL cell suspension into pre-labelled 2 mL cryovials using a serological pipette.
7. Place cryovials into a room temperature CoolCell or Mr Frosty container filled to the mark with 2-propanol.
8. Transfer freezing container to a −70° C. freezer for 24-72 hours.
9. Transfer cryovials to a vapor phase nitrogen storage.
10. Record the location and details of the frozen cells batch.

Preparation of THP-1 Cells and Cell Differentiation (Day 1) Note: Prepare 1×THP-1 24-well plate per test item (control, reference or sample). Each plate requires a minimum of 7 PMA treated wells and 2 non-treated wells. Example plate layout as illustrated below.

TABLE 18
24-Well Plate.

| | 1 | 2 | 3 | 4 | 5 | 6 |
|---|---|---|---|---|---|---|
| A | Empty | Empty | Empty | Empty | Empty | No |
| | | | | | | PMA |
| | | | | | | cells |
| B | PMA | PMA | PMA | PMA | PMA | No |
| | treated | treated | treated | treated | treated | PMA |
| | cells | cells | cells | cells | cells | cells |
| C | PMA | PMA | Empty | Empty | Empty | Empty |
| | treated | treated | | | | |
| | cells | cells | | | | |
| D | Empty | Empty | Empty | Empty | Empty | Empty |

Plate layout includes contingency wells.
1. Pre-warm complete RPMI (c-RPMI) media in a water bath set to 37° C.
2. Remove cells from incubator, visually inspect for signs of contamination and check cells under the microscope.
   Do not proceed in case of contamination. Do not proceed if majority of cells are attached to the culture vessel surface. In such case discard the culture and thaw another vial of working cell bank.
3. Pipette the cell suspension up and down a few times to mix cells and take out small volume of cells for cell counting.
4. Prepare 1 in 2 dilution of the cell suspension in 0.4% trypan blue. Ensure proper mixing of diluted cell suspension by gently mixing with pipette.
5. Prepare C-chips for a total of 2 independent cell counts and determine cell density and viability. Only proceed if cell viability is at least 85%.
6. Prepare cell suspension at 1×10⁶ live cell/mL: centrifuge appropriate volume of cell suspension at 150×g for 5 minutes at RT, discard supernatant and resuspend cell pellets in c-RPMI. Mix well. Prepare minimum 1 mL of cell suspension per well.
7. Add 1 mL of cell suspension to each of two wells labelled “NO PMA” of a 24-well plate (see plate layout).
8. To the remaining cell suspension add 16 pM (1 in 100 dilution from 1.6 mM stock) PMA for a final concentration of approximately 16 nM PMA. Mix well.
9. Add 1 mL PMA treated cell suspensions per well to min of 7 wells on each plate (see plate layout).
10. Incubate cells at 37±1° C., 5±1% CO₂ for 16-24 hrs.

### Infection of THP-1 Cells and Time Course (Day 2)

Preparation of samples and controls. All manipulations of wells containing PMA treated THP-1 cells should be handled with care. Media should be aspirated or dispensed by tilting the plate at approximately a 45 degree angle. Pipette tips should not graze the surface of the well during aspiration or dispensing steps.
1. Retrieve one vial of wild type L. monocytogenes/reference standard or drug product sample.
2. Thaw vial at room temperature for up to 10 min and ensure that the sample is completely thawed.
3. Vortex and transfer cell suspension to a respectively labelled 2 mL microfuge tubes (1 mL). Record the exact volume transferred.
4. Centrifuge cells at 14500×g for 2 min at RT
5. Carefully discard supernatant and resuspend pellet with RT c-RPMI. Volume of medium should be equal to the volume of test item initially transferred in step 3.
6. Prepare dilutions of bacteria using c-RPMI to a final concentration of 1.0×10⁶ CFU/mL. The final volume at this concentration should be approximately 15 mL.
   Follow to the next Section immediately as L. monocytogenes can grow in c-RPMI

Infection of THP-1 cells with L. monocytogenes.
1. Remove one 24-well plate from the incubator.
2. Confirm THP-1 cells adherence under microscope and confirm distinction between cells treated with PMA (remain adherent when shaken lightly) and untreated (exhibit fluidity when shaken lightly).
3. Aspirate media from wells containing PMA treated cells and add 1 mL of the bacteria prepared in step 7 of “Preparation of sample and controls.”
4. Observe plates on a microscope to ensure that THP-1 cells are still adhering to the surface of the wells.
5. Incubate plate at 37±1° C., 5±1% CO₂ for 2 h 3 min.
6. Perform viability testing of test item prepared in “Preparation of samples and controls” following “Viability testing procedure—p-2 time point.”

Viability testing procedure—p-2 time point. Ensure that all agar plates are sufficiently dry prior to initiation of viability testing.
1. Use test item diluted to 1.0×10⁶ CFU/mL as described (Infection of THP 1 Cells and Time Course (Day 2)).
2. Serially dilute the bacterial suspension following the table:

| | bacterial suspension | PBS | total volume | dilution |
|---|---|---|---|---|
| dilution 1 | 100 μL stock | 900 μL | 1000 μL | 1 in 10 |
| dilution 2 | 100 μL dilution 1 | 900 μL | 1000 μL | 1 in 100 |
| dilution 3 | 100 μL dilution 2 | 900 μL | 1000 μL | 1 in 1000 |

3. Spread 100 μL of each dilution onto appropriate BHI agar plates. Make 3 agar plates for each dilution (i.e. in total 9 agar plates are produced per test item for p-2 time point).
   Use agar BHI plates for wild type L. monocytogenes control
   Use agar BM+chloramphenicol for ADXS11-001 test items
4. Each plate is allowed to absorb liquid and dry with its lid on for at least 15 min before being inverted and placed in an incubator at 35-38° C. without CO₂ for 16-24 hours.

Stopping the Infection.
1. Prepare c-RPMI containing 20 μg/mL Gentamicin.
2. After 2 hours, remove plate containing wild type or sample from incubator.
3. Remove L. monocytogenes containing media from each well using a pipette.
4. Carefully dispense 1 mL per well of prepared c-RPMI containing 20 μg/mL Gentamicin, with slow addition against the side of the well to avoid disruption.
5. Return plate to Incubator at 37±1° C., 5±1% CO₂ for 45 min.
6. Remove c-RPMI containing 20 μg/ml Gentamicin from each well using a pipette.
7. Wash cells carefully by addition of 1 mL of c-RPMI without gentamicin per well (slowly adding against the side of well to avoid disruption of monolayer).
8. Remove c-RMPI from each well using a pipette.
9. Carefully dispense 1 mL of c-RPMI (without gentamicin) to each well by slowly adding against the side of well to avoid disruption and return plate to incubator set to 37±1° C., 5±1% CO₂ for at least 5 min. End of incubation time is designated as p0.

Detection of intracellular L. monocytogenes growth—p0.
1. Pick one well to use for the time point “p0” collection.
   Ensure the layer of PMA treated THP-1 cells in each well is consistent and that minimal to no cells were dislodged and removed during the previous aspiration and dispensing steps. If any wells are observed to have significant THP-1 cells loss, mark well to know not to use them.
2. Remove the c-RPMI from the selected well by aspiration with pipette.
3. Dispense 1 mL of sterile water into the well. Dislodge the THP-1 cells from surface of the well by pipetting up and down. Transfer entire contents into 2 mL centrifuge tube Observe under a microscope to confirm that cells have successfully been removed. If a significant portion of THP-1 cells remain, utilize a portion of the water previously transferred to a 2 mL tube to dislodge the cells by pipetting up and down. Transfer contents back into 2 mL tube and confirm under microscope that THP-1 cells have been removed.
4. Return plate to 37±1° C., 5±1% CO₂ incubator until the next time point is ready to be collected
5. Vortex cell lysates for at least 1 min to release intracellular bacteria and conduct viability testing as per “Viability testing procedure—p0/p3 time points.”

Detection of Intracellular L. monocytogenes Growth—p3.
1. Remove plate from the incubator after 3 hours from the time designated as p0 (“Stopping the infection,” step 9).
2. Pick one well to use for the time point “p3” collection.
   Ensure the layer of PMA treated THP-1 cells in each well is consistent and that minimal to no cells were dislodged and removed during the previous aspiration and dispensing steps. If any wells are observed to have significant THP-1 cells loss, mark well to know not to use them.
3. Remove the c-RPM1 from the selected well by aspiration with pipette.
4. Dispense 1 mL of sterile water into the well. Dislodge the THP-1 cells from surface of the well by pipetting up and down. Transfer entire contents into 2 mL centrifuge tube. Observe well under a microscope to confirm that calls have successfully been removed. If a significant portion of THP-1 cells remain, utilize a portion of the water previously transferred to a 2 mL tube to dislodge the cells by pipetting up and down. Transfer contents back into 2 mL tube and confirm under microscope that THP-1 cells have been removed.
5. Vortex cell lysates for at least 1 min to release intracellular bacteria and conduct viability testing as per “Viability testing procedure—p0/p3 time points.”

Viability Testing Procedure—p0/p3 Time Points.
1. Use test item lysate generated in:
   For p0: “Detection of intracellular L. monocytogenes growth—p0, step 5
   For p3: “Detection of intracellular L. monocytogenes growth—p3, step 5
2. Serially dilute the bacterial suspension following the table:
   bacterial suspension PBS total volume dilution

| | bacterial suspension | PBS | total volume | dilution |
|---|---|---|---|---|
| dilution 1 | 100 μL stock | 900 μL | 1000 μL | 1 in 10 |
| dilution 2 | 100 μL dilution 1 | 900 μL | 1000 μL | 1 in 100 |
| dilution 3 | 100 μL dilution 2 | 900 μL | 1000 μL | 1 in 1000 |

3. Spread 100 L of each dilution onto appropriate BHI agar plates. Make 3 agar plates for each dilution (i.e. in total 9 agar plates are produced per test item for p-2 time point).
   Use agar BHI plates for wild type L. monocytogenes control.
   Use agar BHI+chloramphenicol for ADXS11-001 test items.
4. Each plate will be allowed to absorb liquid and dry with its lid on for at least 15 min before being inverted and placed in an incubator at 35-38° C. without CO₂ for 16-24 hours.

Control Plates.
1. For each assay occasion prepare following negative control agar plates:

Uninoculated:
3×BHI agar+100 pg/mL streptomycin
3×BHI agar+25 pg/mL chloramphenicol

Inoculated:
3×BHI agar+100 pg/mL streptomycin inoculated with 100 μL PBS
3×BHI agar+25 pg/mL chloramphenicol inoculated with 100 μL PBS
2. For each assay occasion prepare following positive control agar plates:
   2×BHI agar+100 pg/mL streptomycin inoculated with 10 μL wild type L. monocytogenes at 1×10⁶ CFU/mL.
   2×BHI Agar+25 pg/mL Chloramphenicol inoculated with 10 L reference standard at 1×10⁶ CFU/mL.
3. Plates are incubated alongside assay agar plates in 35-38° C. without CO₂ for 16-24 hours.

Colony Counting (Day 3)
1. After 16-24 hours, remove plates from incubator
   Note: ensure that all L. monocytogenes cell types (wild type, reference standard and sample) at each time point are incubated for the same duration).
2. Total number of colony forming units will be counted manually and recorded for each plate of each dilution in worksheet.

Calculations
1. Use only colony counts with values within 40-600 for subsequent calculations. Minimum 2 colony counts per dilution must be within range to perform necessary calculations. If more than two plates have colony counts outside of the 40-600 range, repeat the entire assay using an adjusted dilution at p0 and/or p3 time points (“Viability testing procedure—p0/p3 time points,” step 2).
2. For each time point calculate the CFU/mL value:

$$\frac{CFU}{mL} = \frac{\sum colony count \times 10 \times dilution factor}{number of dilutions used}$$

3. Perform log₁₀ transformation of all calculated CFU/mL values.
4. Plot the data with log₁₀ CFU/mL on y-axis and time on x-axis.

### Assay Acceptance Criteria

1. No evidence of bacterial growth on all negative control agar plates.
2. Colonies must be present on all positive control agar plates.
3. Mean log 10 (CFU/mL) for control calculated at p-2 is within 6±0.5
4. % CV*between valid colony count values for triplicate agar plates≤30.

*CV=[(standard deviation/mean)×100]
5. Calculate the Cell line performance (CLP) parameter following the formula:

$$Cell line performance ( CLP ) = \frac{P - 2 reference}{P 0 reference} + \frac{P - 2 wt}{P 0 wt}$$

For all valid assay runs CLP≥3. CLP values are subject to tracking. P-2, p0=mean CFU/mL value for time point p-2 and p0, respectively.
6. Calculate the reference response against control using following formula:

$$Reportable result reference ( RRS ) = \frac{P 3 wt}{P 0 wt} + \frac{P 3 reference}{P 0 reference}$$

For all valid assay runs RRS≥2.0. RRS values are subject to tracking. P3, p0=mean CFU/mL value for time point p3 and p0, respectively.

### Reportable Results

1. For each sample calculate the reportable result using following formula:

$$Reportable result reference ( RR ) = \frac{P 3 wt}{P 0 wt} + \frac{P 3 sample}{P 0 sample}$$

Report to 1 decimal places (d.p.).
2. Assess the result against specification.

### Example 5. Validation of THP-1-Based Infectivity Assays for Listeria monocytogenes

A general overview of the method is provided in Example 4.

TABLE 19
Summary.

| Parameter | Acceptance Criteria | Result | Outcome |
|---|---|---|---|
| Intra-assay | Reportable result for all samples (% CV ≤ 25%) | 8.3% | Pass |
| precision | | | |
| Inter-assay | Reportable result for all samples (% CV ≤ 50%) | 18.6% | Pass |
| precision | | | |
| (Intermediate) | | | |
| Specificity | Two-way ANOVA analysis from wild type and | P-2 CFU/mL values | Pass |
| | reference samples will be applied to determine | equivalent for control and | |
| | similarity of growth patterns at p-2, p0, and p3 | reference. No evidence for | |
| | time points. At p-2 time point mean Infectivity is | equivalency at p0 and p3 | |
| | expected to be equivalent between control and | | |
| | reference. No evidence for equivalence is expected | | |
| | at p0 and p3 time points. | | |
| Robustness | Reportable result for all samples (% CV ≤ 25%) | 17.4% and | Pass |
| confirmation | | 9.1% | |

### Methodology

Assay takes 3 days to complete. On day 1, THP-1 cells were plated in 24-well tissue culture plates at 1×10⁶ live cells/mL (one plate per test item—see above (Infection of THP 1 Cells and Time Course (Day 2)). Only cells with viability greater than 85% were used and passage number for the culture was limited to P32. THP-1 cells were then treated with PMA solution to stimulate differentiation to macrophages during the overnight incubation.

On the following day differentiation was confirmed visually using a light microscope. Differentiated cells adhere to the well surface and are morphologically distinct from undifferentiated rounded cells remaining in suspension.

Concentration of each test item was adjusted to 1×10⁶ CFU/mL (based on nominal concentration) and further serially diluted to 10⁻¹, 10⁻² and 10⁻³. 100 μL of each dilution was plated on BHI agar plates and incubated for 16-24 hours to allow colony growth. 3 plates were prepared for each dilution. Colonies were then manually counted to produce p-2 CFU/mL values. At this time point the fixed amount of CFU/mL prior to infection were expected to produce 6±0.5 log₁₀ CFU/mL. This assured that the same amount of the test items is used to infect THP-1 cells.

Test items adjusted to 1×10⁶ CFU/mL were also added to differentiated THP-1 cells for 2 hours 3 minutes. During this time L. monocytogenes bacteria entered the THP-1 cells. All bacteria remaining in the culture medium were then killed by addition of gentamicin for 45 minutes. Gentamicin cannot penetrate the cell membrane of THP-1 cells and therefore only extracellular bacteria were removed in this step. THP-1 cells harboring L. monocytogenes were lysed. Lysates were serially diluted to 10⁻¹, 10⁻² and 10⁻³. 100 μL of each dilution was plated on BHI agar plates and incubated for 16-24 hours to allow colony growth. 3 plates were prepared for each dilution. Colonies were then manually counted to produce p0 CFU/mL values. At this time point number of infecting bacterial cells was determined for each test item.

Several wells containing L. monocytogenes infected THP-1 cells were left in the incubator for 3 hours after completion of treatment with Gentamicin. At the end of the incubation time, cells were lysed. Lysates were serially diluted to 10⁻¹, 10⁻² and 10⁻³. 100 μL of each dilution was plated on BHI agar plates and incubated for 16-24 hours to allow colony growth. 3 plates were prepared for each dilution. Colonies were then manually counted to produce p3 CFU/mL values. At this time point, infection progress was determined for each test item.

Control plates were also prepared to evaluate the aseptic technique and identity of test items via antibiotic resistance profile. Control plates were incubated alongside p-2, p0, and p3 BHI agar plates.

### Data Analysis

Each BHI agar plate was manually counted. Each colony equals 1 CFU. Each preparation/lysate dilution (i.e. 10⁻¹, 10⁻² and 10⁻³) gave 3 colony count values (i.e. CFU). It was expected that at least one dilution will produce colony counts within 40-600 colonies/BHI agar plate and with % CV<30%. CFU/mL value was calculated for each test item at p-2, p0, and p3 time points based on the following equation:

$$\frac{CFU}{mL} = \frac{\sum colony count \times 10 \times dilution factor}{number of dilutions used}$$

Log₁₀ (CFU/mL) at p-2 for control was expected to be within 6±0.5 for all valid assay runs.

To evaluate the intracellular growth of each test item the reportable result was calculated using the following equation:

$$Reportable result ( RR ) = \frac{P 3 wt}{P 0 wt} + \frac{P 3 reference}{P 0 reference}$$

Where p3, p0=mean CFU/mL for time point p3 and p0, respectively.
Reportable result is calculated to 1 d.p.
Reportable result for reference standard material is expected to be ≥2.0.
In addition, the permissiveness to infection of differentiated THP-1 cells is measured by calculating the cell liner performance parameter:

$$Cell line performance ( CLP ) = \frac{P - 2 reference}{P 0 reference} + \frac{P - 2 wt}{P 0 wt}$$

Where p-2, p0=mean CFU/mL for time point p-2 and p0, respectively.
Cell line performance parameter was calculated to 0 d.p.
Cell line parameter was expected to be ≥3 for all valid assay runs.

### Methodology for Evaluation of Method Performance Parameters

TABLE 20
Analytical Matrix.

| | Intra-assay precision | | Inter-assay precision + robustness confirmation | | | | |
|---|---|---|---|---|---|---|---|
| Assay no. | A1 | A2 | A3 | A4 | A5 | A6 | A7 |
| Test item 1 | Control | Control | Control | Control | Control | Control | Control |
| Test item 2 | Ref. | Ref. | Ref. | Ref. | Ref. | Ref. | Ref. |
| Test item 3 | Ref. | N/A | N/A | N/A | N/A | Ref. | Ref. |
| Test item 4 | Ref. | N/A | N/A | N/A | N/A | Ref. | Ref. |

| Culture Type | Working Cell Bank (WCB) | |
|---|---|---|
| Assay Status | Same day | Different days and min of 2 groups of analysts |
| | and same | |
| | analyst | |

| Multiplicity | 1.1 | |
|---|---|---|
| of infection | | |
| (MOI) | | |
| Infection time | 2 h | 2 h ± 3 min |

| | |
|---|---|
| THP-1 passage | ≤p32 |

Ref. = references

Intra-assay precision. For the intra-assay precision, data were collected from one assay occasion (A1) consisting of reference material tested in triplicate (n=3) and one control (n=1). The data reflect variability under the same analytical conditions. Reference material (n=3) preparations were prepared and treated independently during the same assay occasion.

Calculations: mean/SD reportable result for reference standard (test item 2 in assay A1-A7)+% CV; n=7.

Specificity. Specificity of the assay was defined as ability of the test system to distinguish growth pattern of control from the reference material/sample.

In order to take into account the effect of time and item on CFU/mL a two-way analysis of variance (ANOVA) was performed with item, time and their interaction as fixed factors and replicate included as a random effect. The interaction effect describes the difference in time course within each item. The data were logarithmically transformed (base 10) prior to analysis.

Following the above ANOVA, the equivalence of each item was compared to control using the Two One Side Tests methodology (TOST). For each comparison the confidence interval for the difference between control mean and item mean was determined. Considering an equivalence interval of (−0.5, 0.5) for the difference between means, a 90% confidence interval for the difference between the two means was determined. If both confidence limits lie within the equivalence interval then the two means were declared equivalent.

Calculations were performed by ENVIGO statistics department using SAS software (version 9.1.3 using Proc GLM).

Robustness confirmation. Based on the findings from a pre-validation study, time of infection of TIP-1 cells was defined as 2 hours+/−3 minutes. To demonstrate that this range has no impact on the reportable result 2 assays were performed using the lower and upper limit of the infection time (A6 and A7). Mean reportable result from assay A1 (n=3) was compared to mean reportable result of assay A6 (n=3) and A7 (n=3). The % CV for A1, A6 and A7 is expected to be ≤25.

TABLE 21
Critical Materials.

| Material | Supplier | Nominal concentration | Lot |
|---|---|---|---|
| ADXS11-001 | Advaxis | 8.8 × 10⁹ CFU/mL | 5265-14-01 |
| (reference material) | | | |
| Wild type L. monocytogenes | Advaxis | 1.7 × 10⁹ CFU/mL | NB89p25 |
| (control) | | | |

### Results

TABLE 22
Assay Acceptance Criteria Evaluation.

| Acceptance | | A1 | A2 | A3 | A4 | A5 | A6 | A7 |
|---|---|---|---|---|---|---|---|---|
| Worksheet reference | | WS/047 | WS/048 | WS/049 | WS/051 | WS/054 | WS/055 | WS/052 |
| Max. | Control (TI-1) p-2 | 13 | 4 | 4 | 7 | 7 | 11 | 10 |
| % CV colony | Control (TI-1) p0 | 2 | 2 | 2/8² | 11/11² | 8/15² | 3 | 5 |
| (530% = | Control (TI-1) p3 | 4 | 3 | 4 | 4 | 5 | 9 | 6 |
| pass) | Reference (TI-2) p-2 | 15 | 11 | 5 | 4 | 7 | 1 | 9 |
| | Reference (TI-2) p0 | 6 | 15 | 9 | 3 | 8 | 12 | 8 |
| | Reference (TI-3) p3 | 4 | 5 | 5 | 2 | 5 | 5 | 24 |
| | Reference (TI-3) p-2 | 10 | | | | | 11 | 2 |
| | Reference (TI-3) p0 | 3 | | | | | 6 | 6 |
| | Reference (TI-3) p3 | 4 | | | | | 6 | 10 |
| | Reference (TI-4) p-2 | 6 | | | | | 7 | 4 |
| | Reference (TI-4) p0 | 6 | | | | | 5 | 11 |
| | Reference (TI-4) p3 | 4 | | | | | 8 | 5 |

| | | | | | | | |
|---|---|---|---|---|---|---|---|
| CLP (≥3 = pass)¹ | 32 | 48 | 31 | 55 | 27 | 21 | 11 |
| RRS (≥2.0 = pass)³ | 4.2 | 2.8 | 3.0 | 3.6 | 4.1 | 3.4 | 4.5 |
| p-2 log10(CFU/mL) control | 6.1 | 6.1 | 6.1 | 6.1 | 6.1 | 6.2 | 6.0 |
| (5.5-6.5 = pass) | | | | | | | |
| Negative control plates | Growth | Growth | Growth | Growth | Growth | Growth | Growth |
| | absent | absent | absent | absent | absent | absent | absent |
| Positive control plates | Growth | Growth | Growth | Growth | Growth | Growth | Growth |
| | present | present | present | present | present | present | present |

¹CLP reported as mean for assays with more than one reference test item.
²% CV for 10⁻² and 10⁻³ dilutions respectively.
³RRS reported as mean for assays with more than one reference test item.

TABLE 23
Intra-Assay Precision.

| Parameter | Test item 2 | Test item 3 | Test item 4 |
|---|---|---|---|
| Worksheet reference | WS/047 | WS/047 | WS/047 |
| Assay number | A1 | A1 | A1 |
| Test item type | Reference | Reference | Reference |
| Reportable results | 4.2 | 3.9 | 4.6 |
| (control: test item 1A1) | | | |

| | |
|---|---|
| SD/% CV | 0.35/8.3% |
| Validation acceptance | Pass |
| (% CV ≤ 25%) | |

TABLE 24
Intra-Assay Precision.

| Parameter | Test item 2 | Test item 3 | Test item 4 |
|---|---|---|---|
| Worksheet reference | WS/055 | WS/055 | WS/055 |
| Assay number | A6 | A6 | A6 |
| Test item type | Reference | Reference | Reference |
| Reportable results | 3.8 | 2.7 | 3.6 |
| (control: test item 1 A1) | | | |

| | |
|---|---|
| SD/% CV | 0.59/17.4% |
| Validation acceptance | Pass |
| (% CV ≤ 25%)¹ | |

¹Robustness confirmation. Assay A6 evaluated increased infection time (2 h ± 3 min)

TABLE 25
Intra-Assay Precision.

| Parameter | Test item 2 | Test item 3 | Test item 4 |
|---|---|---|---|
| Worksheet reference | WS/052 | WS/052 | WS/052 |
| Assay number | A7 | A7 | A7 |
| Test item type | Reference | Reference | Reference |
| Reportable results | 4.8 | 4.0 | 4.5 |
| (control: test item 1A1) | | | |

| | |
|---|---|
| SD/% CV | 0.40/9.1% |
| Validation acceptance | Pass |
| (% CV ≤ 25%)¹ | |

¹Robustness confirmation. Assay A7 evaluated decreased infection time (2 h ± 3 min).

TABLE 26
Inter-Assay Precision (Intermediate).

| Parameter | A1 | A2 | A3 | A4 | A5 | A6 | A7 |
|---|---|---|---|---|---|---|---|
| Worksheet reference | WS/047 | WS/048 | WS/049 | WS/051 | WS/054 | WS/055 | WS/052 |
| Test item type | Ref. | Ref. | Ref. | Ref. | Ref. | Ref. | Ref. |
| Reportable results (control: test | 4.2 | 2.8 | 3.0 | 3.6 | 4.1 | 3.8 | 4.8 |
| item 1 from respective assay) | | | | | | | |

| | |
|---|---|
| % CV | 0.70/18.6% |
| Validation acceptance | Pass |
| (% CV ≤ 50%)¹ | |

TABLE 27
Specificity.

| | Time | | | | |
|---|---|---|---|---|---|
| Comparison | point | Difference | Lower CL | Upper CL | Equivalence |
| Item 2 vs control | −2 | −0.12 | −0.18 | −0.07 | Yes |
| Item 2 vs control | 0 | −1.55 | −1.60 | −1.51 | No |
| Item 2 vs control | 3 | −2.18 | −2.23 | −2.13 | No |
| Item 3 vs control | −2 | −0.14 | −0.19 | −0.10 | Yes |
| Item 3 vs control | 0 | −1.68 | −1.73 | −1.64 | No |
| Item 3 vs control | 3 | −2.28 | −2.32 | −2.23 | No |
| Item 4 vs control | −2 | −0.10 | −0.15 | −0.06 | Yes |
| Item 4 vs control | 0 | −1.62 | −1.67 | −1.57 | No |
| Item 4 vs control | 3 | −2.28 | −2.32 | −2.23 | No |

TABLE 28

| Parameters | | Test item 2 | Test item 3 | Test item 4 |
|---|---|---|---|---|
| Worksheet reference | | WS/047 | WS/047 | WS/047 |
| Assay number | | A1 | A1 | A1 |
| 2-way ANOVA vs. | p-2 | Means equivalent | Means equivalent | Means equivalent |
| test item 1 (control) | p0 | Means non-equivalent | Means non-equivalent | Means non-equivalent |
| | p3 | Means non-equivalent | Means non-equivalent | Means non-equivalent |

| | |
|---|---|
| Outcome | Pass |

TABLE 29
Colony Counts.

| | Test | | p-2 | | | | p0 | | | | p3 | | | |
|---|---|---|---|---|---|---|---|---|---|---|---|---|---|---|
| Assay | Item | | | CFU | CFU | CFU | | CFU | CFU | CFU | | CFU | CFU | CFU |
| No. | No. | Type | dilution | 1 | 2 | 3 | dilution | 1 | 2 | 3 | dilution | 1 | 2 | 3 |
| A1 | 1 | control | 1/1000 | 118 | 154 | 132 | 1/100 | 368 | 375 | 385 | 1/1000 | 429 | 396 | 403 |
| | 2 | reference | 1/1000 | 120 | 89 | 101 | 1/10 | 100 | 103 | 112 | 1/10 | 262 | 267 | 281 |
| | 3 | reference | 1/1000 | 86 | 104 | 100 | 1/10 | 77 | 76 | 80 | 1/10 | 207 | 217 | 225 |
| | 4 | reference | 1/1000 | 110 | 108 | 98 | 1/10 | 94 | 93 | 84 | 1/10 | 206 | 220 | 221 |
| A2 | 1 | control | 1/1000 | 113 | 120 | 121 | 1/100 | 332 | 329 | 319 | 1/1000 | 353 | 340 | 362 |
| | 2 | reference | 1/1000 | 86 | 82 | 100 | 1/10 | 50 | 59 | 44 | 1/20 | 190 | 204 | 184 |
| A3 | 1 | control | 1/1000 | 135 | 146 | 140 | 1/100 | 375 | 325 | 363 | 1/1000 | 409 | 382 | 408 |
| | | | | | | | 1/100 | 51 | 57 | N/A¹ | | | | |
| | 2 | reference | 1/1000 | 85 | 82 | 91 | 1/10 | 79 | 95 | 89 | 1/10 | 269 | 249 | 271 |
| A4 | 1 | control | 1/1000 | 120 | 137 | 14 | 1/100 | 410 | 403 | 337 | 1/1000 | 499 | 485 | 460 |
| | | | | | | | 1/1000 | 61 | 51 | 50 | | | | |
| | 2 | reference | 1/1000 | 102 | 103 | 109 | 1/10 | 70 | 70 | 67 | 1/10 | 196 | 202 | 195 |
| A5 | 1 | control | 1/1000 | 125 | 110 | 123 | 1/100 | 310 | 351 | 303 | 1/1000 | 325 | 337 | 357 |
| | | | | | | | 1/1000 | 40 | 52 | 41 | | | | |
| | 2 | reference | 1/1000 | 81 | 90 | 80 | 1/10 | 100 | 93 | 110 | 1/10 | 208 | 227 | 226 |
| A6 | 1 | control | 1/1000 | 155 | 127 | 155 | 1/100 | 202 | 215 | 210 | 1/1000 | 212 | 207 | 180 |
| | 2 | reference | 1/1000 | 74 | 75 | 74 | 1/10 | 55 | 53 | 66 | 1/10 | 141 | 140 | 152 |
| | 3 | reference | 1/1000 | 89 | 72 | 78 | 1/10 | 52 | 52 | 47 | 1/10 | 190 | 181 | 170 |
| | 4 | reference | 1/1000 | 78 | 89 | 85 | 1/10 | 60 | 59 | 55 | 1/10 | 168 | 150 | 143 |
| A7 | 1 | control | 1/1000 | 96 | 105 | 118 | 1/100 | 97 | 95 | 105 | 1/1000 | 115 | 107 | 120 |
| | 2 | reference | 1/1000 | 89 | 75 | 86 | 1/10 | 72 | 67 | 79 | 1/10 | 144 | 158 | 224 |
| | 3 | reference | 1/1000 | 78 | 81 | 79 | 1/10 | 58 | 64 | 65 | 1/10 | 174 | 196 | 162 |
| | 4 | reference | 1/1000 | 85 | 80 | 87 | 1/10 | 75 | 67 | 60 | 1/10 | 167 | 180 | 166 |

¹colony count outside of permitted range (40-600 colonies).

1. A method of assessing attenuation or infectivity of a test Listeria strain, comprising:
(a) infecting differentiated THP-1 cells with the test Listeria strain, wherein the THP-1 cells have been differentiated into macrophages prior to infecting with the test Listeria strain;
(b) lysing the THP-1 cells and plating the lysate on agar; and
(c) counting the Listeria that have multiplied inside the THP-1 cells by growth on the agar.

2. The method of claim 1, further comprising differentiating the THP-1 cells into macrophages using phorbol 12-myristate 13-acetate (PMA) prior to step (a).

3. The method of claim 1, wherein step (a) comprises infecting the differentiated THP-1 cells at a multiplicity of infection (MOI) of 1:1.

4. The method of claim 1, further comprising killing Listeria not taken up by the THP-1 cells in between steps (a) and (b).

5. The method of claim 4, wherein the killing is performed using an antibiotic, optionally wherein the antibiotic is gentamicin.

6. The method of claim 1, wherein step (b) is performed at 0 hours post-infection.

7. The method of claim 1, wherein step (b) is performed at 3 hours post-infection.

8. The method of claim 1, further comprising comparing uptake and intracellular growth of the test Listeria strain with a wild type Listeria strain and/or a reference sample.

9. The method of claim 1, wherein the test Listeria strain is a Listeria monocytogenes strain.

10. The method of claim 1, wherein the test Listeria strain is a recombinant Listeria strain comprising a nucleic acid comprising a first open reading frame encoding a fusion polypeptide, wherein the fusion polypeptide comprises a PEST-containing peptide fused to a disease-associated antigenic peptide.

11. The method of claim 10, wherein the PEST-containing peptide is listeriolysin O (LLO) or a fragment thereof, and the disease-associated antigenic peptide is selected from the group consisting of a Human Papilloma virus (HPV) protein E7 Prostate Specific Antigen (PSA), a chimeric Her2 antigen, and Her2/neu chimeric antigen, or a fragment thereof.

12. The method of claim 10, wherein the recombinant Listeria strain is an attenuated Listeria monocytogenes strain comprising a deletion of or inactivating mutation in prfA, wherein the nucleic acid is in an episomal plasmid and comprises a second open reading frame encoding a D133V PrfA mutant protein.

13. The method of claim 10, wherein the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of listeriolysin O (LLO).

14. A method of assessing attenuation or infectivity of a test bacteria strain, comprising:
(a) differentiating THP-1 cells;
(b) infecting the differentiated THP-1 cells with the test bacteria strain, wherein the infecting comprises:
(i) inoculating the differentiated THP-1 cells with the test bacteria strain;
(ii) incubating the test bacteria strain with the differentiated THP-1 cells for 1-5 hours to form infected THP1 cells;
(iii) removing extracellular bacteria from the infected THP-1 cells; and
(iv) incubating the infected THP-1 cells in growth media for 0-10 hours;
(c) lysing the infected THP-1 cells to form a lysate;
(d) plating the lysate or a dilution of the lysate on a plate containing media capable of supporting growth of the bacteria; and
(e) counting colony forming units of the bacteria on the plate.

15. The method of claim 14, wherein the step of infecting the differentiated THP-1 cells is at a multiplicity of infection (MOI) of 1:1.

16. The method of claim 14, wherein the step of removing extracellular bacteria comprises adding an antibiotic effective against the bacteria, optionally wherein the antibiotic is gentamicin.

17. The method of claim 14, wherein the infected THP-1 cells are incubated in growth media for 0, 1, 3, or 5 hours.

18. The method of claim 14, wherein the test bacteria strain is an L. monocytogenes strain.

19. The method of claim 1, wherein the test Listeria strain is a recombinant Listeria strain comprising a nucleic acid comprising a first open reading frame encoding a fusion polypeptide, wherein the fusion polypeptide comprises a PEST-containing peptide fused to two or more disease-associated antigenic peptides.

20. The method of claim 19, wherein the PEST-containing peptide comprises a bacterial secretion signal sequence, and the fusion polypeptide further comprises a ubiquitin protein fused to a carboxy-terminal antigenic peptide, wherein the PEST-containing peptide, the two or more disease-associated antigenic peptides, the ubiquitin, and the carboxy-terminal antigenic peptide are arranged in tandem from the amino-terminal end to the carboxy-terminal end of the fusion polypeptide.
